(19) United States
(12) Patent Application Publication
(10) Pub. No.: US 2003/0215914 A1
(43) Pub. Date: Nov. 20, 2003

(54) Structure for presenting desired peptide sequences

(76) Inventors: Erwin Houtzager, Amerongen (NL); Irma Maria Caecilia Vijn, Bennekom (NL); Peter Christiaan Sijmons, Amsterdam (NL)

Correspondence Address: TRASK, BRITT, & ROSSA, P.O. Box 2550, Salt Lake City, UT, 84110, US

(21) Appl. No.: 10/316,194

(22) Filed: Dec. 10, 2002

Related U.S. Application Data
(63) Continuation-in-part of application No. 10/016,516, filed on Dec. 10, 2001.

(51) Int. Cl. C07K 14/435; C07H 21/04; C12P 21/02; C12N 5/06

(52) U.S. Cl. 435/69.1; 435/320.1; 435/325; 530/350; 536/23.5

(57) ABSTRACT

Means and methods for generating binding peptide associated with a suitable core region are disclosed, the resulting proteinaceous molecule and uses thereof. A solution to the problems associated with the use of binding molecules over their entire range of use. Binding molecules can be designed to accommodate extreme conditions of use such as extreme temperatures or pH. Alternatively, binding molecules can be designed to respond to very subtle changes in the environment.

### CROSS-REFERENCE TO RELATED APPLICATION

This application claims priority as a continuation-in-part of U.S. patent application Ser. No. 10/016,516 filed Dec. 10, 2001, the contents of which are incorporated by this reference herein in its entirety.

### TECHNICAL FIELD

The invention relates to methods and means for providing binding molecules with improved properties, be it in binding or other properties, as well as the novel binding molecules themselves. The invention further relates to methods applying these molecules in all their versatility.

### BACKGROUND

In modem biotechnology, one of the most promising and in a number of cases proven applications relies on affinity of proteinaceous molecules for all kinds of substances and/or targets. Proteinaceous binding molecules have been applied in purification of substances from mixtures, in diagnostic assays for a wide array of substances, as well as in the preparation of pharmaceuticals, etc.

Typically, naturally occurring proteinaceous molecules, such as immunoglobulins (or other members of the immunoglobulin superfamily) as well as receptors and enzymes have been used. Also peptides derived from such molecules have been used.

The use of existing (modified) natural molecules of course provides a limited source of properties that evolution has bestowed on these molecules. This is one of the reasons that these molecules have not been applied in all the areas where their use can be envisaged. Also, because evolution always results in a compromise between the different functions of the naturally occurring binding molecules, these molecules are not optimized for their envisaged use. Typically, the art has moved in the direction of altering binding properties of existing (modified) binding molecules. In techniques such as phage display of (single chain) antibodies almost any binding specificity can be obtained. However, the binding regions are all presented in the same context. Thus, the combination of binding region and its context is often still not optimal, limiting the use of the proteinaceous binding molecules in the art.

### SUMMARY OF THE INVENTION

The present invention provides a versatile context for presenting desired affinity regions. The present invention provides a structural context that is designed based on a common structural element (called a core structure) that has been identified herein to occur in numerous binding proteins. This so called “common core” has now been produced as a novel proteinaceous molecule that can be provided with one or more desired affinity regions.

This proteinaceous structure does not rely on any amino acid sequence, but only on common structural elements. It can be adapted by providing different amino acid sequences and/or amino acid residues in sequences for the intended application. It can also be adapted to the needs of the particular affinity region to be displayed. The invention thus also provides libraries of both structural contexts and affinity regions to be combined to obtain an optimal proteinaceous binding molecule for a desired purpose.

Thus, the invention provides a synthetic or recombinant proteinaceous molecule comprising a binding peptide and a core, the core comprising a β-barrel comprising at least 5 strands, wherein the β-barrel comprises at least two β-sheets, wherein at least one of the β-sheets comprises three of the strands and wherein the binding peptide is a peptide connecting two strands in the β-barrel and wherein the binding peptide is outside its natural context. We have identified this core structure in many proteins, ranging from galactosidase to human (and, for example, camel) antibodies with all kinds of molecules in between. Nature has apparently designed this structural element for presenting desired peptide sequences. We have now produced this core in an isolated form, as well as many variants thereof that still have the same or similar structural elements. These novel structures can be used in all applications where other binding molecules are used and even beyond those applications as explained herein. The structure comprising one affinity region (desired peptide sequence) and two β-sheets forming one β-barrel is the most basic form of the invented proteinaceous binding molecules. (proteinaceous means that they are in essence amino acid sequences, but that side chains and/or groups of all kinds may be present; it is of course possible, since the amino acid sequence is of less relevance for the structure to design other molecule of non proteinaceous nature that have the same orientation is space and can present peptidic affinity regions; the orientation in space is the important parameter). The invention also discloses optimized core structures in which less stable amino acids are replaced by more stable residues (or vice versa) according to the desired purpose. Of course other substitutions or even amino acid sequences completely unrelated to existing structures are included since; once again, the important parameter is the orientation of the molecule in space. According to the invention it is preferred to apply a more advanced core structure than the basic structure, because both binding properties and structural properties can be designed better and with more predictive value. Thus, the invention preferably provides a proteinaceous molecule according the invention wherein the β-barrel comprises at least 5 strands, wherein at least of the sheets comprises 3 of the strands, more preferably a proteinaceous molecule according to the invention, wherein the β-barrel comprises at least 6 strands, wherein at least two of the sheets comprises 3 of the strands. β-barrel wherein each of the sheets comprises at least 3 strands are sufficiently stable while at the same time providing sufficient variation possibilities to adapt the core/affinity region (binding peptide) to particular purposes. However, suitable characteristics can also be found with cores that comprise fewer strands per sheet. Thus, variations wherein one sheet comprises only two strands are within the scope of the present invention. In an alternative embodiment the invention provides a proteinaceous molecule according to the invention wherein the β-barrel comprises at least 7 strands, wherein at least one of the sheets comprises 4 of the strands. Alternatively the invention provides a proteinaceous molecule according to the invention, wherein the beta-barrel comprises at least 8 strands, wherein at least one of the sheets comprises 4 of the strands. In another embodiment a proteinaceous molecule according to the invention, wherein the β-barrel comprises at least 9 strands, wherein at least one of the sheets comprises 4 of the strands is provided. In the core structure there is a more open side where nature displays affinity regions and a more closed side, where connecting sequences are present. Preferably, at least one affinity region is located at the more open side.

Thus the invention provides a proteinaceous molecule according to the invention, wherein the binding peptide connects two strands of the β-barrel on the open side of the barrel. Although the location of the desired peptide sequence (affinity region) may be anywhere between two strands, it is preferred that the desired peptide sequence connects the two sheets of the barrel. Thus, the invention provides a proteinaceous molecule according to the invention, wherein the binding peptide connects the at least two β-sheets of the barrel. Although one affinity region may suffice it is preferred that more affinity regions are present to arrive at a better binding molecule. Preferably, these regions are arranged such that they can cooperate in binding (e.g., both on the open side of the barrel). Thus, the invention provides a proteinaceous molecule according to the invention, which comprises at least one further binding peptide. A successful core element in nature is the one having three affinity regions and three connecting regions. This core in its isolated form is a preferred embodiment of the present invention. However, because of the versatility of the presently invented binding molecules, the connecting sequences on the less open side of the barrel can be used as affinity regions as well. This way a very small “bispecific” binding molecule is obtained. Thus, the invention provides a proteinaceous molecule according the invention, which comprises at least 4 binding peptides. “Bispecific” means that the binding molecule has the possibility to bind to two target molecules (the same or different). The various strands in the core are preferably encoded by a single open reading frame. The loops connecting the various strands may have any type of configuration. So as not to unduly limit the versatility of the core it is preferred that loops connect strands on the same side of the core, i.e., and N-terminus of strand (a) connects to a C-terminus of strand (b) on either the closed side or the open side of the core. Loops may connect strands in the same β-sheet or cross-over to the opposing β-sheet. A preferred arrangement for connecting the various strands in the core are given in the examples and the FIGS., and in particular FIG. 1. Strands in the core may be in any orientation (parallel or antiparallel) with respect to each other. Preferably, the strands are in the configuration as depicted in FIG. 1.

The present invention optimizes binding molecules both in the binding properties and the structural properties (such as stability under different circumstances (temperature, pH, etc.), the antigenicity, etc.). This is done, according tot the invention by taking at least one nucleic acid according to the invention (encoding a proteinaceous binding molecule according to the invention) and mutating either the encoded structural regions or the affinity regions or both and testing whether a molecule with desired binding properties and structural properties has been obtained. Thus, the invention provides a method for identifying a proteinaceous molecule with an altered binding property, comprising introducing an alteration in the core of proteinaceous molecules according to the invention, and selecting from the proteinaceous molecules, a proteinaceous molecule with an altered binding property, as well as a method for identifying a proteinaceous molecule with an altered structural property, comprising introducing an alteration in the core of proteinaceous molecules according to the invention, and selecting from the proteinaceous molecules, a proteinaceous molecule with an altered structural property. These alterations can vary in kind, an example being a post-translational modification. The person skilled in the art can design other relevant mutations.

As explained the mutation would typically be made by mutating the encoding nucleic acid and expressing the nucleic acid in a suitable system, which may be bacterial, eukaryotic or even cell-free. Once selected, one can, of course, use other systems than the selection system.

The invention also provides methods for producing nucleic acids encoding proteinaceous binding molecules according to the invention, such as a method for producing a nucleic acid encoding a proteinaceous molecule capable of displaying at least one desired peptide sequence comprising providing a nucleic acid sequence encoding at least a first and second structural region separated by a nucleic acid sequence encoding the desired peptide sequence or a region where such a sequence can be inserted and mutating the nucleic acid encoding the first and second structural regions to obtain a desired nucleic acid encoding the proteinaceous molecule capable of displaying at least one desired peptide sequence and preferably a method for displaying a desired peptide sequence, providing a nucleic acid encoding at least a two β-sheets, the , the β-sheets forming a β-barrel, the nucleic acid comprising a region for inserting a sequence encoding the desired peptide sequence, inserting a nucleic acid sequence comprising a desired peptide sequence, and expressing the nucleic acid whereby the βsheets are obtainable by a method as described above. The invention further provides the application of the novel binding molecules in all fields where binding molecules have been envisaged until today, such as separation of substances from mixtures, typically complex biological mixtures, such as body fluids or secretion fluids, such as blood or milk, or serum or whey.

Of course pharmaceutical uses and diagnostic uses are clear to the person skilled in the art. In diagnostic uses labels may be attached to the molecules of the invention. In pharmaceutical uses other moieties can be coupled to the molecules of the invention. In both cases this may be chemically or through recombinant fusion. Diagnostic applications and pharmaceutical applications have been described in the art in great detail for conventional binding molecules. For the novel binding molecules according tot the invention no further explanation is necessary for the person skilled in the art. Gene therapy in a targeting format is one of the many examples wherein a binding molecule according to the invention is provided on the gene delivery vehicle (which may be viral (adenovirus, retrovirus, adeno associated virus or lentivirus, etc.) or non viral (liposomes and the like). Genes to be delivered are well known in the art.

The gene delivery vehicle can also encode a binding molecule according to the invention to be delivered to a target, possibly fused to a toxic moiety. Conjugates of toxic moieties to binding molecules are also well known in the art and are included for the novel binding molecules of the invention.

### BRIEF DESCRIPTION OF THE DRAWINGS

FIG. 1 is a schematic 3D-topology of scaffold domains. Eight example topologies of protein structures that can be used for the presentation of antigen binding sites are depicted. The basic core beta elements are the nominated in example A. This basic structure contains 9 beta-elements positioned in two plates. One beta-sheets contains elements 1, 2, 6 and 7 and the other contains elements 3, 4, 5, 8 and 9. The loops that connect the beta-elements are also depicted. Bold lines arc connecting loops between beta-elements that are in top position while dashed lines indicate connecting loops that are located in bottom position. A connection that starts dashed and ends solid indicates a connection between a bottom and top part of beta-elements. The numbers of the beta-elements depicted in the diagram correspond to the numbers and positions mentioned in FIGS. 1 and 2. A: 9 beta element topology: for example all antibody light and heavy chain variable domains and T-cell receptor variable domains. B: 8 beta element topology: for example interleukin-4 alpha receptor (1IAR): 7a beta element topology: for example immunoglobulin killer receptor 2dl2 (2DLI) D: 7b beta element topology: for example E-cadherin domain (1IFF5). E: 6a beta strand topology F: 6b beta element topology: for example Fc epsilon receptor type alpha (1J88) G: 6c beta element topology: for example interleukin-1 receptor type-1 (1GOY) H: 5 beta element topology.

FIG. 2 is a modular Affinity & Scaffold Transfer (MAST) Technique. Putative antigen binding proteins that contain a core structure as described here can be used for transfer operations. In addition, individual or multiple elements or regions of the scaffold or core structures can also be used for transfer actions. The transfer operation can occur between structural identical or comparable scaffolds or cores that differ in amino acid composition. Putative affinity regions can be transferred from one scaffold or core to another scaffold or core by for example PCR, restriction digestions, DNA synthesis or other molecular techniques. The results of such transfers are depicted here in a schematic diagram. The putative (coding) binding regions from molecule A (top part, affinity regions) and the scaffold (coding) region of molecule B (bottom part, framework regions) can be isolated by molecular means. After recombination of both elements a new molecule appears (hybrid structure) that has binding properties of molecule A and scaffold properties of scaffold B.

FIG. 3 is a domain notification of immunoglobular structures. The diagram represents the topologies of protein structures consisting of respectively 9, 7 and 6 beta-elements (indicated 1-9 from N-terminal to C-terminal). Beta elements 1, 2, 6 and 7 and elements 3, 4, 5, 8 and 9 form two beta-sheets. Eight loops (L1-L8) are responsible for the connection of all beta-elements. Loop 2, 4, 6 and 8 are located at the top site of the diagram and this represents the physical location of these loops in example proteins. The function of loops 2, 4 and 8 in light and antibody variable domains is to bind antigens, known as CDR regions. The position of L6 (also marked with a patterned region) also allows antigen binding activity, but has not been indicated as a binding region. L2, L4, L6, L8 are determined as affinity region 1 (AR1), AR2, AR3 and AR4 respectively. Loops 1, 3, 5 and 7 are located at the opposite site of the proteins.

FIG. 4 is A) Schematic overview of vector CM126 B) Schematic overview of vector CM126.

FIG. 5 illustrates solubilization of inclusion bodies of iMab100 using heat (60° C.) Lanes: Molecular weight marker (1), isolated inclusion bodies of iMab100 (2), solubilized iMab100 upon incubation of inclusion bodies in PBS pH 8+1% Tween-20 at 60° C. for 10 minutes.

FIG. 6 Purified iMab variants containing 6-, 7- or 9 beta-sheets. Lanes: Molecular weight marker (1), iMab1300 (2), iMab1200 (3), iMab701 (4), iMab101 (5), iMab900(6), iMab122 (7), iMab1202 (8 ), iMab1602 (9), iMab1302 (10), iMab116 (11), iMab111 (12), iMab100 (13).

FIG. 7 is Stability of iMab100 at 95° C. Purifed iMab100 incubated for various times at 95° C. was analyzed for binding to ELK (squares) lysozyme (circles).

FIG. 8 Stability of iMab100 at 20° C. Purified iMab100 incubated for various times at 20° C. was analyzed for binding to ELK (squares) or chicken lysozyme (circles).

FIG. 9 A. far UV CD spectrum (205-260 nm) of iMab100 at 20° C., 95° C., and again at 20° C. iMab100 was dissolved in 1×PBS, pH 7.5. B. iMab111, far UV spectrum determined at 20° C., (partially) denatured at 95° C., and refolded at 20° C., compared to the iMab100 spectrum at 20° C. C. iMab116, far UV spectrum determined at 20° C., (partially) denatured at 95° C., and refolded at 20° C., compared to the iMab100 spectrum at 20° C. D. iMab1202, far UV spectrum determined at 20° C., (partially) denatured at 95° C., and refolded at 20° C., compared to the iMab100 spectrum at 20° C. E. iMab1302, far UV spectrum determined at 20° C., (partially) denatured at 95° C., and refolded at 20° C., compared to the iMab100 spectrum at 20° C. F. iMab1602, far UV spectrum determined at 20° C., (partially) denatured at 95° C., and refolded at 20° C., compared to the iMab100 spectrum at 20° C. G. iMab101, far UV spectrum determined at 20° C., partially) denatured at 95° C., and refolded at 20° C. H. iMab1200, far UV spectrum determined at 20° C., (partially) denatured at 95 ° C., and refolded at 20° C. I. iMab701, far UV spectrum determined at 20° C., (partially) denatured at 95° C., and refolded at 20° C. J. Overlay of native (undenatured) 9 strand iMab scaffolds. K. Overlay of native (undenatured) 7 strand iMab scaffolds. L. Far UV CD spectra of iMab100 and a V_{HH} (courtesy Kwaaitaal M, Wageningen University and Research, Wageningen, the Netherlands).

FIG. 10 is Schematic overview of PCR isolation of CDR3 for MAST.

FIG. 11 Amplification Cow derived CDR3 regions2% Agarose—TBE gel. Lane 1. 1 microgram Llama cDNA cyst+, PCR amplified with primers 8 and 9.Lane 2. 1 microgram Llama cDNA cyst-, PCR amplified with primers 8 and 9.Lane 3. 25 bp DNA step ladder (Promega).Lane 4. 0.75 microgram Cow cDNA PCR amplified with primers 299 and 300.Lane 5. 1.5 microgram Cow cDNA PCR amplified with primers 299 and 300.Lane 6. 0.75 microgram Cow cDNA PCR amplified with primers 299 and 301.Lane 7. 1.5 microgram Cow cDNA PCR amplified with primers 299 and 301.Lane 8. 50 bp Gene Ruler DNA ladder (MBI Fermentas).

FIG. 12 Lysozyme binding activity measured with ELISA of iMab100. Several different solutions were tested in time for proteolytic activity on iMab100 proteins. Test samples were diluted 100 times in FIGS. A) and C) while samples were 1000 times diluted in FIGS. B) and D). A) and B) show lysozyme activity while C) and D) show background activity.

### DETAILED DESCRIPTION

The present invention relates to the design, construction, production, screening and use of proteins that contain one or more regions that may be involved in molecular binding. The invention also relates to naturally occurring proteins provided with artificial binding domains, re-modeled natural occurring proteins provided with extra structural components and provided with one or more artificial binding sites, re-modeled naturally occurring proteins disposed of some elements (structural or others) provided with one or more artificial binding sites, artificial proteins containing a standardized core structure motif provided with one or more binding sites. All such proteins are called VAPs (Versatile Affinity Proteins) herein. The invention further relates to novel VAPs identified according to the methods of the invention and the transfer of binding sites on naturally occurring proteins that contain a similar core structure. 3D modeling or mutagenesis of such natural occurring proteins can be desired before transfer in order to restore or ensure antigen binding capabilities by the affinity regions present on the selected VAP. Further, the invention relates to processes that use selected VAPs, as described in the invention, for purification, removal, masking, liberation, inhibition, stimulation, capturing, etc., of the chosen ligand capable of being bound by the selected VAP(s).

Ligand Binding Proteins

Many naturally occurring proteins that contain a (putative) molecular binding site comprise two functionally different regions: The actual displayed binding region and the region(s) that is (are) wrapped around the molecular binding site or pocket, called the scaffold herein. These two regions are different in function, structure, composition and physical properties. The scaffold structures ensure a stable 3 dimensional conformation for the whole protein, and act as a steppingstone for the actual recognition region.

Two functional different classes of ligand binding proteins can be discriminated. This discrimination is based upon the presence of a genetically variable or invariable ligand binding region. In general, the invariable ligand binding proteins contain a fixed number, a fixed composition and an invariable sequence of amino acids in the binding pocket in a cell of that species. Examples of such proteins are all cell adhesion molecules, e.g., N-CAM and V-CAM, the enzyme families, e.g., kinases and proteases and the family of growth receptors, e.g. EGF-R, bFGF-R. In contrast, the genetically variable class of ligand binding proteins is under control of an active genetic shuffling-, mutational or rearrangement mechanism enabling an organism or cell to change the number, composition and sequence of amino acids in, and possibly around, the binding pocket. Examples of these are all types of light and heavy chain of antibodies, B-cell receptor light and heavy chains and T-cell receptor alpha, beta, gamma and delta chains. The molecular constitution of wild type scaffolds can vary to a large extent. For example, Zinc finger containing DNA binding molecules contain a totally different scaffold (looking at the amino acid composition and structure) than antibodies although both proteins are able to bind to a specific target.

Scaffolds and Ligand Binding Domains

Antibodies Obtained via Immunizations

The class of ligand binding proteins that express variable (putative) antigen binding domains has been shown to be of great value in the search for ligand binding proteins. The classical approach to generate ligand binding proteins makes use of the animal immune system. This system is involved in the protection of an organism against foreign substances. One way of recognizing, binding and clearing the organism of such foreign highly diverse substances is the generation of antibodies against these molecules. The immune system is able to select and multiply antibody producing cells that recognize an antigen. This process can also be mimicked by means of active immunizations. After a series of immunizations antibodies may be formed that recognize and bind the antigen. The possible number of antibodies with different affinity regions that can be formed due to genetic rearrangements and mutations exceeds the number of 10⁴⁰. However, in practice, a smaller number of antibody types will be screened and optimized by the immune system. The isolation of the correct antibody producing cells and subsequent immortalization of these cells or, alternatively, cloning of the selected antibody genes directly, antigen-antibody pairs can be conserved for future (commercial and non-commercial) use.

The use of antibodies obtained this way is restricted only to a limited number of applications. The structure of animal antibodies is different than antibodies found in human. The introduction of animal derived antibodies in humans, for example, for medical applications will almost certainly cause immune responses adversely affecting the effect of the introduced antibody (e.g., HAMA reaction). As it is not allowed to actively immunize men for commercial purposes, it is not or only rarely possible to obtain human antibodies this way. Because of these disadvantages methods have been developed to bypass the generation of animal specific antibodies. One example is the removal of the mouse immune system and the introduction of the human immune system in such mouse. All antibodies produced after immunization are of human origin. However, the use of animals has also a couple of important disadvantages. First, animal care has a growing attention from ethologists, investigators, public opinion and government. Immunization belongs to a painful and stressful operation and must be prevented as much as possible. Second, immunizations do not always produce antibodies or do not always produce antibodies that contain required features such as binding strength, antigen specificity, etc. The reason for this can be multiple: the immune system nissed by co-incidence such a putative antibody; the initially formed antibody appeared to be toxic or harmful; the initially formed antibody also recognizes animal specific molecules and consequently the cells that produce such antibodies will be destroyed; or the epitope cannot be mapped by the immune system (this can have several reasons).

Otherwise Obtained Antibodies

It is clear, as discussed above, that immunization procedures may result in the formation of ligand binding proteins but their use is limited, inflexible and uncontrollable. The invention of methods for the bacterial production of antibody fragments (Skerra and Pluckthun, 1988; Better et al., 1988) provided new powerful tools to circumvent the use of animals and immunization procedures. It is has been shown that cloned antibody fragments, (frameworks, affinity regions and combinations of these) can be expressed in artificial systems, enabling the modulation and production of antibodies and derivatives (Fab, V_{L}, V_{H}, scFv and V_{HH}) that recognize a (putative) specific target in vitro. New efficient selection technologies and improved degeneration strategies directed the development of huge artificial (among which human) antibody fragment libraries. Such libraries potentially contain antibodies fragments that can bind one or more ligands of choice. These putative ligand specific antibodies can be retrieved by screening and selection procedures. Thus, ligand binding proteins of specific targets can be engineered and retrieved without the use of animal immunizations.

Other Immunoglobulin Superfamily Derived Scaffolds

Although most energy and effort is put in the development and optimization of natural derived or copied human antibody derived libraries, other scaffolds have also been described as successful scaffolds as carriers for one or more ligand binding domains. Examples of scaffolds based on natural occurring antibodies encompass minibodies (Pessi et al., 1993), Camelidae V_{HH} proteins (Davies and Riechmann, 1994; Hamers-Casterman et al., 1993) and soluble V_{H} variants (Dimasi et al., 1997; Lauwereys et al., 1998). Two other natural occurring proteins that have been used for affinity region insertions are also member of the immunoglobulin superfamily: the T-cell receptor chains (Kranz et al., WO Patent 0148145) and fibronectin domain-3 regions (Koide U.S. Pat. No. 6,462,189; Koide et al., 1998). The two T-cell receptor chains can each hold three affinity regions according to the inventors while for the fibronectin region the investigators described only two regions.

Non-Immunoglobulin Derived Scaffolds

Besides immunoglobulin domain derived scaffolds, non-immunoglobulin domain containing scaffolds have been investigated. All proteins investigated contain only one protein chain and one to four affinity related regions. Smith and his colleagues (1998) reported the use of knottins (a group of small disulfide bonded proteins) as a scaffold. They successfully created a library based on knottins that had 7 mutational amino acids. Although the stability and length of the proteins are excellent, the low number of amino acids that can be randomized and the singularity of the affinity region make knottin proteins not very powerful. Ku and Schultz (1995) successfully introduced two randomized regions in the four-helix-bundle structure of cytochrome b₅₆₂. However, selected binders were shown to bind with micromolar K_{d} values instead of the required nanomolar or even better range. Another alternate framework that has been used belongs to the tendamistat family of proteins. McConnell and Hoess (1995) demonstrated that alpha-amylase inhibitor (74 amino acid beta-sheet protein) from Streptomyces tendae could serve as a scaffold for ligand binding libraries. Two domains were shown to accept degenerated regions and function in ligand binding. The size and properties of the binders showed that tendamistats could function very well as ligand mimickers, called mimetopes. This option has now been exploited. Lipocalin proteins have also been shown to be successful scaffolds for a maximum of four affinity regions (Beste et al., 1999; Skerra, 2000 BBA; Skerra, 2001 RMB). Lipocalins are involved in the binding of small molecules like retinoids, arachidonic acid and several different steroids. Each lipocalin has a specialized region that recognizes and binds one or more specific ligands. Skerra (2001) used the lipocalin RBP and lipocalin BBP to introduce variable regions at the site of the ligand binding domain. After the construction of a library and successive screening, the investigators were able to isolate and characterize several unique binders with nanomolar specificity for the chosen ligands. It is currently not known how effective lipocalins can be produced in bacteria or fungal cells. The size of lipocalins (about 170 amino acids) is pretty large in relation to V_{HH} chains (about 100 amino acids), which might be too large for industrial applications.

Core Structure Development

In commercial industrial applications, it is very interesting to use single chain peptides, instead of multiple chain peptides because of low costs and high efficiency of such peptides in production processes. One example that could be used in industrial applications is the V_{HH} antibodies. Such antibodies are very stable, can have high specificities and are relatively small. However, the scaffold has evolutionarily been optimized for an immune dependent function but not for industrial applications. In addition, the highly diverse pool of framework regions that are present in one pool of antibodies prevents the use of modular optimization methods. Therefore a new scaffold was designed based on the favorable stability of V_{HH} proteins.

3D-modelling and comparative modeling software was used to design a scaffold that meets the requirements of versatile affinity proteins (VAPs).

However, at this moment it is not yet possible to calculate all possible protein structures, protein stability and other features, since this would cost months of computer calculation capacity. Therefore we test the most promising computer designed scaffolds in the laboratory by using display techniques, such as phage display or the like. In this way it is possible to screen large numbers of scaffolds in a relatively short time.

Immunoglobulin-like (ig-like) folds are very common throughout nature. Many proteins, especially in the animal kingdom, have a fold region within the protein that belongs to this class. Reviewing the function of the proteins that contain an ig-like fold and reviewing the function of this ig-like fold within that specific protein, it is apparent that most of these domains, if not all, are involved in ligand binding. Some examples of ig-like fold containing proteins are: V-CAM, immunoglobulin heavy chain variable domains, immunoglobulin light chain variable domains, constant regions of immunoglobulins, T-cell receptors, fibronectin, reovirus coat protein, beta-galactosidase, integrins, EPO-receptor, CD58, ribulose carboxylase, desulphoferrodoxine, superoxide likes, biotin decarboxylase and P53 core DNA binding protein. A classification of most ig-like folds can be obtained from the SCOP database (Murzin A. G et al., 1995; http://scop.mrc-lmb.cam.ac.uk/scop) and from CATH (Orengo et al, 1997; http://www.biochem.ucl.ac.uk/bsm/cath_new/index.html). SCOP classifies these folds as: all beta proteins, with an immunoglobulin-like beta-sandwich in which the sandwich contains 7 strands in 2 sheets although some members that contain the fold have additional strands. CATH classifies these folds as: Mainly beta proteins with an architecture like a sandwich in an immunoglobulin-like fold designated with code 2.60.40. In structure databases like CE (Shindyalov et al.1998; http:/cl.sdsc.edu/ce.htm), VAST (Gibrat et al.,1996; http://www.ncbi.nlm.nih.gov/Structure/VAST/vast.shtml) and FSSP (Holm et al, 1998; http://www.ebi.ac.uk/dali/fssp) similar classifications are used.

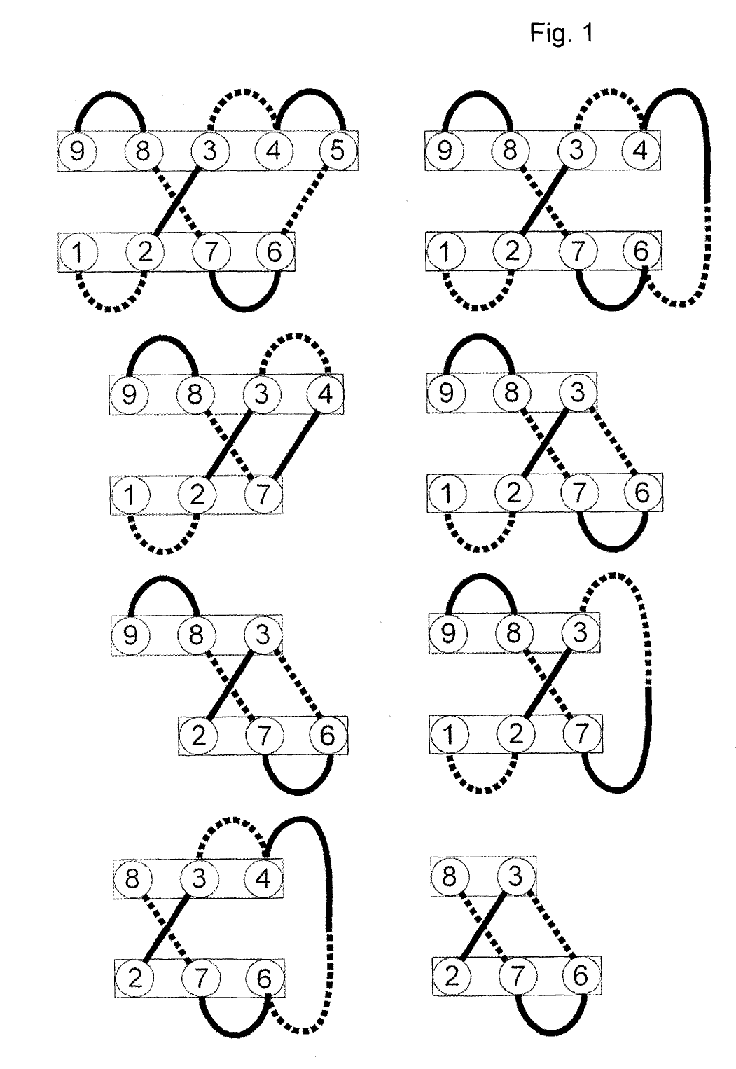

Projection of these folds from different proteins using software of Cn3D (NCBI; http://www.ncbi.nlm.nih.gov/Structure/CN3D/cn3d.shtml), InsightII (MSI; http://www.accelrys.com/insight) and other structure viewers, showed that the ig-like folds have different sub-domains. A schematic projection of the structure is depicted in FIG. 1. The most conserved structure was observed in the centre of the folds, named the core. The core structures hardly vary in length and have a relative conserved spatial constrain, but they were found to vary to a large degree in both sequence and amino acid composition. On both sides of the core, sub-domains are present. These are called connecting loops. These connecting loops are extremely variable as they can vary in amino acid content, sequence, length and configuration. The core structure is therefore designated as the far most important domain within these proteins. The number of beta-elements that form the core can vary between 7 and 9 although 6 stranded core structures might also be of importance. All beta-elements of the core are arranged in two beta-sheets. Each beta-sheet is build of anti-parallel oriented beta-elements. The minimum number of beta-elements in one beta-sheet that was observed was 3 elements. The maximum number of beta-element in one sheet that was observed was 5 elements, although it can not be excluded that higher number of beta-elements might be possible. Connecting loops connect the beta-elements on one side of the barrel. Some connections cross the beta-sheets while others connect beta-elements that are located within one beta-sheet. Especially the loops that are indicated as L2, L4, L6 and L8 are used in nature for ligand binding and are therefore preferred sites for the introduction or modification of binding peptide/affinity regions. The high variety in length, structure, sequences and amino acid compositions of the L 1, L3, L5 and L7 loops clearly indicates that these loops can also be used for ligand binding, at least in an artificial format.

Amino acid side chains in the beta-elements that form the actual core that are projected towards the interior of the core, and thus fill the space in the centre of the core, can interact with each other via H-bonds, covalent bonds (cysteine bridges) and other forces, and determine the stability of the fold. Because amino acid composition and sequence of the residues of the beta-element parts that line up the interior were found to be extremely variable, it was concluded that many other sequence formats and can be created.

In order to obtain the basic concept of the structure as a starting point for the design of new types of proteins containing this ig-like fold, projections of domains that contain ig-like folds were used. Insight II, Cn3D and Modeller programs were used to determine the minimal elements and lengths. In addition, only C-alpha atoms of the structures were projected because these described the minimal features of the folds. Minor differences in spatial positions (coordinates) of these beta elements were allowed.

PDB files representing the coordinates of the C-alpha atoms of the core of ig-like folds were used for the development of new 9, 8, 7 , 6 and 5 beta-elements containing structures. For 8 stranded structures beta element 1 or 9 can be omitted but also elements 5 or 6 can be omitted. Thus, an eight stranded core preferably comprises elements 2-8, and either 1 or 9. Another preferred eight stranded core comprises elements 1-4, 7-9, and either strand 5 or strand 6. For 7 stranded structures, 2 beta-elements can be removed among which combinations of element 1 and 9, 1 and 5, 6 and 9, 9 and 5 and, elements 4 and 5. The exclusion of elements 4 and 5 is preferred because of spatial constrains. Six stranded structures lack preferably element 1, 4 and 5 or 4, 5 and 9 but also other formats were analyzed with Insight and Modeller and shown to be reliable enough for engineering purposes.

Multiple primary scaffolds were constructed and pooled. All computer designed proteins are just an estimated guess. One mutation or multiple amino acid changes in the primary scaffold may make it a successful scaffold or make it function even better than predicted. To accomplish this the constructed primary scaffolds are subjected to a mild mutational process by PCR amplification that includes error-prone PCR, such as unequimolar dNTP concentration, addition of manganese or other additives, or the addition of nucleotide analogues, such as dITP (Spee et al., 1993) or dPTP (Zaccolo et al., 1996) in the reaction mixture which can ultimately change the amino acid compositions and amino acid sequences of the primary scaffolds. This way new (secondary) scaffolds are generated.

In order to test the functionality, stability and other characteristics required or desired features of the scaffolds, a set of known affinity regions, such as 1MEL for binding lysozyme and 1BZQ for binding RNase were inserted in the primary modularly constructed scaffolds. Functionality, heat and chemical stability of the constructed VAPs were determined by measuring unfolding conditions. Functionality after chemical or heat treatment was determined by binding assays (ELISA), while temperature induced unfolding was measured using a circular dichroism (CD) polarimeter. Phage display techniques were used to select desired scaffolds or for optimization of scaffolds.

Initial Affinity Regions for Library Construction

In the present invention, new and unique affinity regions are required. Affinity regions can be obtained from natural sources, degenerated primers or stacked DNA triplets. All of these sources have certain important limitations as described above. In our new setting we designed a new and strongly improved source of affinity regions which have less restrictions, can be used in modular systems, are extremely flexible in use and optimization, are fast and easy to generate and modulate, have a low percentage of stop codons, have an extremely low percentage of frameshifts and wherein important structural features will be conserved in a large fraction of the new formed clones and new structural elements can be introduced.

The major important affinity region (CDR3) in both light and heavy chain in normal antibodies has a average length between 11 (mouse) and 13 (human) amino acids. Because in such antibodies the CDR3 in light and heavy chain cooperatively function as antigen binder, the strength of such a binding is a result of both regions together. In contrast, the binding of antigens by V_{HH} antibodies (Camelidae) is a result of one CDR3 region due to the absence of a light chain. With an estimated average length of 16 amino acids these CDR3 regions are significantly longer than regular CDR3 regions (Mol. Immunol. Bang Vu et al., 1997, 34, 1121-1131). It can be emphasized that long or multiple CDR3 regions have potentially more interaction sites with the ligand and can therefore be more specific and bind with more strength. Another exception are the CDR3 regions found in cow (Bos taurus) (Berens et al., 1997). Although the antibodies in cow consists of a light and a heavy chain, their CDR3 regions are much longer than found in mouse and humans and are comparable in length found for camelidae CDR3 regions. Average lengths of the major affinity region(s) should preferably be about 16 amino acids. In order to cover as much as possible potentially functional CDR lengths the major affinity region can vary between 1 and 50 or even more amino acids. As the structure and the structural classes of CDR3 regions (like for CDR1 and CDR2) have not been clarified and understood it is not possible to design long affinity regions in a way that the position and properties of crucial amino acids are correct. Therefore, most libraries were supplied with completely degenerated regions in order to find at least some correct regions.

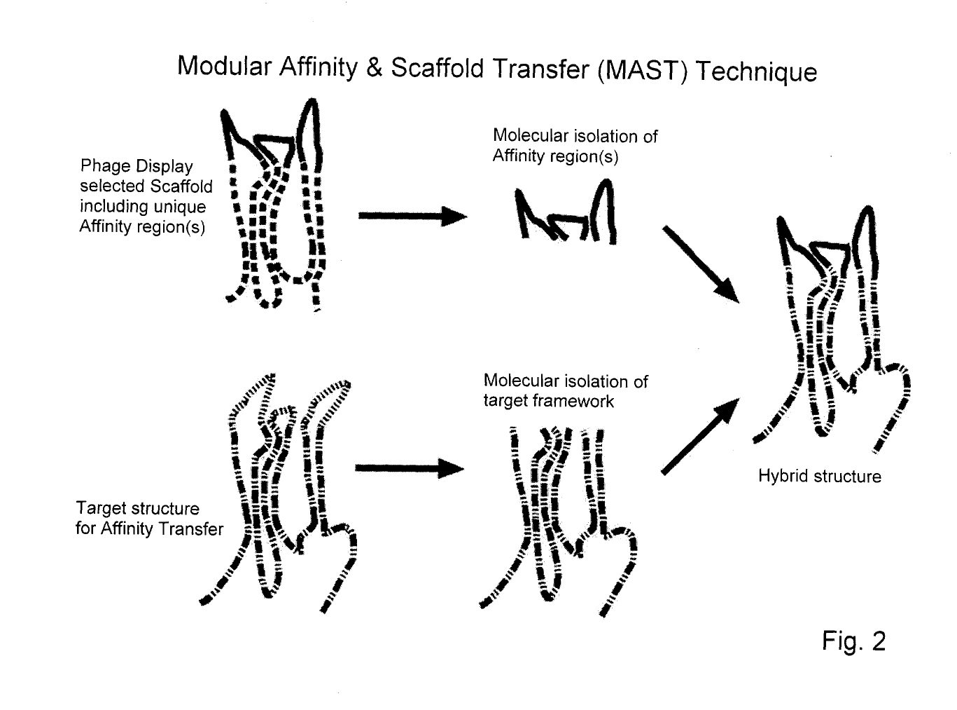

In the invention, we describe the use of natural occurring camelidae V_{HH} CDR3 as well as bovine derived V_{H} CDR3 regions as a template for new affinity regions, but of course other CDR regions (e.g., CDR1 and CDR2) as well as other varying sequences that corresponds in length might be used. CDR3 regions were amplified from mRNA coding for V_{HH} antibodies originating from various animals of the camelidae group or from various other animals containing long CDR3 regions by means of PCR techniques. Next, this pool of about 10⁸ different CDR3 regions, which differ in the coding for amino acid composition, amino acid sequence, putative structural classes and length, is subjected to a mutational process by PCR as described above. The result is that most products will differ from the original templates and thus contain coding regions that potentially have different affinity regions. Other very important consequences are that the products keep their length, the pool keeps their length distribution, a significant part will keep structural important information while others might form non-natural classes of structures, the products do not or only rarely contain frame shifts and the majority of the products will lack stop codons. These new affinity regions can be cloned into the selected scaffolds by means of the Modular Affinity and Scaffold Transfer technology (MAST). This technique is based on the fact that all designed and constructed scaffolds described above have a modular structure such that all loops connecting the beta-strands can be easily replaced by other loops without changing the overall structure of the VAP (see FIG. 2) The newly constructed library can be subjected to screening procedures similar to the screening of regular libraries known by an experienced user in the field of the art. Thus, further provided is a method for producing a library comprising artificial binding peptides the method comprising providing at least one nucleic acid template wherein the templates encode different specific binding peptides, producing a collection of nucleic acid derivatives of the templates through mutation thereof and providing the collection or a part thereof to a peptide synthesis system to produce the library comprising artificial binding peptides. The complexity of the library increases with increasing number of different templates used to generate the library. In this way an increasing number of different structures used. Thus, preferably at least two nucleic acid templates, and better at least 10 nucleic acid templates are provided. Mutations can be introduced using various means and methods. Preferably, the method introduces mutations by changing bases in the nucleic acid template or derivative thereof. With “derivative” is meant a nucleic acid comprising at least one introduced mutation as compared to the temple. In this way the size of the affinity region is not affected. Suitable modification strategies include amplification strategies such as PCR strategies encompass for example unbalanced concentrations of dNTPs (Cadwell et al., (1992); Leung et al., (1989) 1; Kuipers, (1996), the addition of dITP (Xu et al. (1999); Spee et al. (1993; Kuipers, (1996), dPTP (Zaccolo et al., 5 (1996)), 8-oxo-dG (Zaccolo et al., (1996)), Mn²⁺(Cadwell et al., (1992); Leung et al., (1989) 1, , Xu et al., (1999)), polymerases with high misincorporation levels (Mutagene®, Stratagene). Site specific protocols for introducing mutations can of course also be used, however, the considerable time and effort to generate a library using such methods would opt against a strategy solely based on site directed mutagenizes. Hybrid strategies can of course be used. Mutation strategies comprising dITP and/or dPTP incorporation during elongation of a nascent strand are preferred since such strategies are easily controlled with respect to the number of mutations that can be introduced in each cycle. The method does not rely on the use of degenerate primers to introduce complexity. Therefore, in one embodiment, the amplification utilizes non-degenerates primers. However, (in part) degenerate primers can be used thus also provided is a method wherein at least one non-degenerate primer further comprises a degenerate region. The methods for generating libraries of binding peptides is especially suited for the generation of the above mentioned preferred larger affinity regions. In these a larger number of changes can be introduced while maintaining the same of similar structure. Thus, preferably at least one template encodes a specific binding peptide having an affinity region comprising at least 14 amino acids and preferably at least 16 amino acids. Though non consecutive regions can be used in this embodiment of the invention it is preferred that the region comprises at least 14 consecutive amino acids. When multiple templates are used it is preferred that the regions comprise an average length of 24 amino acids.

Method for generating a library of binding peptides may favorably be combined with core regions of the invention and method for the generation thereof For instance, once a suitable binding region is selected a core may be designed or selected to accommodate the particular use envisaged. However, it is also possible to select a particular core region, for reasons of the intended use of the binding peptide. Subsequently libraries having the core and the mentioned library of binding peptides may be generated. Uses of such libraries are of course manifold. Alternatively, combinations of strategies may be used to generate a library of binding peptides having a library of cores. Complexities of the respective libraries can of course be controlled to adapt the combination library to the particular use. Thus, in a preferred embodiment at least one of the templates encodes a proteinaceous molecule according to the invention. The mentioned peptide, core and combination libraries may be used to select proteinaceous molecules of the invention, thus herein is further provided a method comprising providing a potential binding partner for a peptide in the library of artificial peptides and selecting a peptide capable of specifically binding to the binding partner from the library. A selected proteinaceous molecule obtained using the method is of course also provided. To allow easy recovery and production of selected proteinaceous molecule it is preferred that at least the core and the binding peptide is displayed on a replicative package comprising nucleic acid encoding the displayed core/peptide proteinaceous molecule. Preferably, the replicative package comprises a phage, such as used in phage display strategies. Thus, also provided is a phage display library comprising at least one proteinaccous molecule of the invention. As mentioned above, the method for generating a library of binding peptides can advantageously be adapted for core regions. Thus, also provided is a method for producing a library comprising artificial cores the method comprising providing at least one nucleic acid template wherein the templates encode different specific cores, producing a collection of nucleic acid derivatives of the templates through mutation thereof and providing the collection or a part thereof to a peptide synthesis system to produce the library of artificial cores. Preferred binding peptides libraries are derived from templates comprising CDR3 regions from cow (Bos Taurus) or camelidae (preferred lama pacos and lama glama).

Affinity Regions (AR's)

Protein-ligand interactions are one of the basic principles of life. All protein-ligand mediated interactions in nature either between proteins, proteins and nucleic acids, proteins and sugars or proteins and other types of molecules are mediated through an interface present at the surface of a protein and the molecular nature of the ligand surface. The very most of protein surfaces that are involved in protein-ligand interactions are conserved throughout the life cycle of an organism. Proteins that belong to these classes are for example receptor proteins, enzymes and structural proteins. The interactive surface area for a certain specific ligand is usually constant. However, some protein classes can modulate their nature of the exposed surface area through e.g., mutations, recombinations or other types of natural genetic engineering programs. The reasons for this action is that their ligands or ligand types can vary to a great extend. Proteins that belong to such classes are e.g., antibodies, B-cell receptors and T-cell receptor proteins. Although there is in principle no difference between both classes of proteins, the speed of surface changes for both classes differ. The first class is mainly sensitive to evolutionary forces (lifespan of the species) while the second class is more sensitive to mutational forces (within the lifespan of the organism).

Single scaffold proteins which are used in applications that require high affinity and high specificity in general require at least one long affinity region or multiple medium length ARs in order to have sufficient exposed amino acid side chains for ligand interactions. Synthetic constructed highly functional long ARs, using primer or triplet fusion strategies, will not be very efficient for reasons as discussed above. Libraries containing such synthetic ARs would either be too low in functionality or too large to handle. The only available source for long ARs is those that can be obtained from animal sources (most often CDR3s in heavy chains of antibodies). Especially cow-derived and camelidae-derived CDR3 regions of respectively Vh chains and Vhh chains are unusual long. The length of these regions is in average above 13 amino acids but 30 amino acids or even more are no exceptions. Libraries constructed with such ARs obtained from immunized animals can be successful for those ligands or ligand domains that are immunological active. Non-immunogenic ligands or ligand domains and ligands that appear to be otherwise silent in immune responsiveness (toxic, self recognition, etc) will not trigger the immune system to produce ligand specific long CDRs. Therefore, long CDRs that mediate the binding of such targets can not or hardly be obtained this way and thus their exist a vacuum in technologies that provides one with specific long ARs that can be used on single scaffold proteins. A comparable conclusion has also been drawn by Muyldermans (Reviews in Molecular Biotechnology 74 (2001) 277-302) who analyzed the use of synthetic ARs on lama Vhh scaffolds.

Isolation of CDR regions, especially CDR3 regions, by means of PCR enables one to use all length variations and use all structural variations present in the available CDR regions. The introduction of minor, mild, medium level or high level random mutations via nucleic acid amplification techniques like, for example, PCR will generate new types of affinity regions. The benefits of such AR pools are that length distributions of such generated regions will be conserved. Also, stop codon introductions and frame shifts will be prevented to a large degree due to the relatively low number of mutations if compared with random primers based methods. Further, depending on the mutational percentage, a significant part or even the majority of the products will code for peptide sequences that exhibit structural information identical or at least partly identical to their original template sequence present in the animal. Due to these mutations altered amino acid sequences will be generated by a vast part of the products and consequently these will have novel binding properties. Binding properties can be altered in respect to the original template not only in strength but also in specificity and selectivity. This way libraries of long AR regions can be Binding specificity and affinity between receptors and ligands is mediated by an interaction between exposed interfaces of both molecules. Protein surfaces are dominated by the type of amino acids present at that location. The 20 different amino acids common in nature each has its own side chain with its own chemical and physical properties. It is the accumulated effect of all amino acids in a certain exposed surface area that is responsible for the possibility to interact with other molecules. Electrostatic forces, hydrophobicity, H-bridges, covalent coupling and other types of properties determine the type, specificity and strength of binding with ligands.

The most sophisticated class of proteins involved in protein-ligand interactions is those of antibodies. An ingenious system has been evolved that controls the location and level mutations, recombinations and other genetic changes within the genes that can code for such proteins. Genetic changing forces are mainly focused to these regions that form the exposed surface area of antibodies that are involved in the binding of putative ligands. The enormous numbers of different antibodies that can be formed (theoretically) indicate the power of antibodies. For example: if the number of amino acids that are directly involved in ligand binding in both the light and heavy chains of antibodies are assumed to be 8 amino acids for each chains (and this is certainly not optimistic) then 20^{2*8} which approximates 10²⁰ (20 amino acids types, 2 chains, 8 residues) different antibodies can be formed. If also indirect effects of nearby located amino acids include and/or increase the actual number of direct interaction amino acids, one ends up with an astronomically large number. Not one organism on earth is ever able to test a1 these or even just a fraction of these combinations in the choice of antibody against the ligand.

Not all amino acids present at the exposed surface area are equally involved in ligand binding. Some amino acids can be changed into other amino acids without any notable- or only minor changes in ligand binding properties. Also, most surface areas of proteins are very flexible and can under the influence of the ligand surface easily remodel resulting in a fit with the ligand surface that would not occur with an inflexible ligand-binding region. Interacting forces as mentioned above between the protein and the ligand can thus steer or catalyze this remodeling. In general, large but limited number of genetic changes together with redundancy in amino acids and the flexible nature of the surface in combination with binding forces can lead to the production of effective ligand binding proteins.

Natural derived antibodies and their affinity regions have been optimized to certain degree, during immune selection procedures. These selections are based upon the action of such molecules in an immune system. Antibody applications outside immune systems can be hindered due to the nature and limitations of the immune selection criteria. Therefore, industrial, cosmetic, research and other applications demand often different properties of ligand binding proteins. The environment in which the binding molecules may be applied can be very harsh for antibody structures, e.g., extreme pH conditions, salt conditions, odd temperatures, etc. Depending on the application CDRs might or might not be transplanted from natural antibodies on to a scaffold. For at least some application unusual affinity regions will be required. Thus, artificial constructed and carefully selected scaffolds and affinity regions will be required for other applications.

Affinity regions present on artificial scaffolds can be obtained from several origins. First, natural affinity regions can be used. CDRs of cDNAs coding for antibody fragments can be isolated using PCR and inserted into the scaffold at the correct position. The source for such regions can be of immunized or non-immunized animals. Second, fully synthetic AR's can be constructed using degenerated primers. Third, semi-synthetic AR's can be constructed in which only some regions are degenerated. Fourth, triplets coding for selected amino acids (monospecific or mixtures) can be fused together in a predetermined fashion. Fifth, natural derived affinity regions (either from immunized or naive animals) which are being mutated during amplification procedures (e.g., NASBA or PCR) by introducing mutational conditions (e.g., manganese ions) or agents (e.g., dITP) during the reaction.

Because for reasons mentioned earlier, immunization based CDRs can be successful but the majority of ligands or ligand domains will not be immunogenic. Artificial affinity regions in combinations with powerful selection and optimization strategies become more and more important if not inevitable. Primer based strategies are not very powerful due to high levels of stop codons, frameshifts, difficult sequences, too large randomizations, relative small number of mutational spots (maximum of about 8 spots) and short randomization stretches (no more than 8 amino acids). The power of non-natural derived AR's depends also on the percentage of AR's that putatively folds correctly, i.e., being able to be presented on the scaffold without folding problems of the AR's or even the scaffold. Hardly any information is currently available about structures and regions that are present in AR's. Therefore the percentage of correctly folded and presented artificial AR's constructed via randomizations, especially long AR's, will be reciprocal with the length of constructed ARs. Insight in CDR and AR structures will most likely be available in the future, but is not available yet. generated with strongly reduced technical or physical problems as mentioned above if compared with synthetic, semi synthetic and natural obtained ARs.

In recent years several new and powerful in vitro mutagenesis methods and agents have been developed. One branch of mutagenizing methods produces mutations independently of the location (in contract to site directed mutagenesis methods). PCR strategies encompass for example unbalanced concentrations of dNTPs (Cadwell et al., (1992) 2,; Leung et al., (1989),; Kuipers, (1996)), the addition of dITP (Xu et al., (1999); Spee et al., (1993); Kuipers 57 (1996)), dPTP (Zaccolo et al., (1996)), 8-oxo-dG (Zaccolo et al., (1996)), Mn2+(Cadwell et al., (1992); Leung et al. (1989);Xu et al., (1999)), polymerases with high misincorporation levels (Mutagene®, Stratagene).

Affinity Maturation

After one or more selection rounds, an enriched population of VAPs is formed that recognizes the ligand selected for. In order to obtain better, different or otherwise changed VAPs against the ligand(s), the VAP coding regions or parts thereof can be the subject of a mutational program as described above due to its modular nature. Several strategies are possible: First, the whole VAP or VAPs can be used as a template. Second, only one or more affinity regions can be mutated. Third framework regions can be mutated. Fourth, fragments throughout the VAP can be used as a template. Of course, iterative processes can be applied to change more regions. The average number of mutations can be varied by changing PCR conditions. This way every desired region can be mutated and every desired level of mutation numbers can be applied independently. After the mutational procedure, the new formed pool of VAPs can be re-screened and re-selected in order to find new and improved VAPs against the ligand(s). The process of maturation can be re-started and re-applied as much rounds as necessary.

The effect of this mutational program is that not only affinity regions 1 and 2 with desired affinities and specificities can be found but also that minor changes in the selected affinity region 3 can be introduced. It has been shown (REF) that mutational programs in this major ligand binding region can strongly increase ligand binding properties. In conclusion, the invention described here is extremely powerful in the maturation phase.

Industrial use of VAPs

The VAPs of the invention can be used in an enormous variety of applications, from therapeutics to antibiotics, from detection reagents to purification modules, etc. In each application, the environment and the downstream applications determines the features that a ligand binding protein should have, e.g., temperature stability, protease resistance, tags, etc. Whatever the choice of the scaffolds is, all have their own unique properties. Some properties can be advantageous for certain applications while others are unacceptable. For large scale industrial commercial uses it is crucial that scaffolds contain a modular design in order to be able to mutate, remove, insert and swap regions easily and quick. Modularity makes it possible to optimize for required properties via standardized procedures and it allows domain exchange programs, e.g., exchange of pre-made cassettes. As optimal modular scaffold genes should meet certain features, they have to be designed and synthetically constructed while it is very unlikely that natural retrieved genes contains such features.

Besides modularity there are several other properties that should be present or just absent in the scaffold gene or protein. All scaffold systems that are based on frameworks that are present in natural proteins inherit also their natural properties. These properties have been optimized by evolutionary forces for the system in which this specific protein acts. Specific properties encompass for example codon usage, codon frequency, expression levels, folding patterns and cysteine bridge formation. Industrial commercial production of such proteins, however, demands optimal expression, translation and folding to achieve economic profits. Not only should the genetic information be compatible and acceptable for the production organism, protein properties should also be optimal for the type of application. Such properties can be heat sensitivity, pH sensitivity, salt concentration sensitivity, proteolytic sensitivity, stability, purification possibilities, and many others.

Thus, to be of practical use in affinity processes, specific binding activity alone is not sufficient. The specific binding agent must also be capable of being linked to a solid phase such as a carrier material in a column, an insoluble bead, a plastic, metal or paper surface or any other useful surface. Ideally, this linkage is achievable without any adverse effects on the specific binding activity. Therefore the linkage is preferably accomplished with regions in the VAP molecule that are relatively remote from the specific affinity regions.

An important embodiment of the invention is an affinity-absorbent material comprising a specific binding agent immobilized on a porous silica or the like, the specific binding agent comprising a selection of VAP molecules.

A particularly important embodiment of the invention is an affinity-absorbent material comprising a special binding agent immobilized on a porous carrier material, such as silica or an inert, rigid polymer or the like, having a pore size of at least 30A but not greater than 1000A, wherein the specific binding agent comprises a selection of VAP molecules. Preferably, the carrier has a pore size of at least 60A. Preferably, the pore size is not greater than 500A, and more preferably, not greater than 300A. The coupling of proteins to support material is widely applied in research and industry (Narayanan and Crane, bas(1990). Polymers as support or carrier material for VAPs include, but are not limited to nylon, vinyl polymers, polyethylene, polypropylene, polystyrene, polym ethylmethacrylate, polyvinylacetate, polytetrafluoroethylene. polyvinylidenefluoride, cellulose, chitin, chitosan , agarose, proteins. Activated (i.e., ready for protein coupling) support materials are commercially available or can be chemcially activated by a person skilled in the art.

The pore size of a carrier medium such as silica or inert polymers can be determined using, e.g., standard size exclusion techniques or other published methods. The nominal pore size is often referred to as the mean pore diameter, and is expressed as a function of pore volume and surface area, as calculated by the Wheeler equation (MPD=(40,000×pore volume)/surface area. The pore volume and surface area can be determined by standard nitrogen absorption methods.

Products in which VAPs can be applied in a way that leaves the VAPs present up to, and also including, the end product, have examples from a very wide range of products. But also in processes where the VAPs are immobilized and preferably can be regenerated for recycled use, the major advantage of VAPs is fully exploited, i.e., the relative low cost of VAPs that makes them especially suitable for large scale applications, for which large quantities of the affinity bodies need to be used. The list below is given to indicate the scope of applications and is in no way limiting. Product or process examples with possible applications in brackets are;

(1) industrial food processing such as the processing of whey, tomato pomace, citrus fruits, etc. or processes related to bulk raw materials of agricultural origin such as the extraction of starch, oil and fats, proteins, fibers, sugars etc. from bulk crops such as, but not limited to; potato, corn, rice, wheat, soybean, cotton, sunflower, sugarbeet, sugarcane, tapioca, rape. Other examples of large process streams are found in the diary-related industries e.g., during cheese and butter manufacturing. As the VAPs can be used in line with existing processing steps and the VAPs do not end up in the final product as a result of their irreversible immobilisation to support-materials, they are exceptionally suited for the large scale industrial environments that are customary in agro-foodprocessing industries. In a more detailed example, the whey fraction that is the result of the cheese manufacturing processes contain a relatively large number of low-abundant proteins that have important biological functions, e.g., during the development of neonates, as natural antibiotics, food-additives etc. Examples of such proteins are lactoferrin, lactoperoxidase, lysozyme, angiogenine, insulin-like growth factors (IGF), insulin receptor, IGF-binding proteins, transforming growth factors (TGF), bound- and soluble TGF-receptors, epidermal growth factor (EGF), EGF-receptor ligands, interleukin-1 receptor antagonist. Another subclass of valuable compounds that can be recovered from whey are the immunoregulatory peptides that are present in milk or colostrum. Also specific VAPs can be selected for the recovery of hormones from whey. Examples of hormones that are present in milk are; prolactin, somatostatin, oxytocin, luteinizing hormone-releasing hormone, thyroid-stimulating hormone, thyroxine, calcitonin, estrogen, progesterone

(2) edible consumer products such as ice-cream, oil-based products such as oils, margarines, dressings and mayonnaise, other processed food products as soups, sauces, pre-fabricated meals, soft-drinks, beer, wine, etc. (preservation and prevention of spoilage, through direct antibiotic activity or selective inhibition of enzymes, protecting sensitive motives during processing, e.g., from enzymes or compounds that influence quality of end products through its presence in an active form, controlled release of flavours and odors, molecular mimics to mask or enhance flavours and odors e.g., masking or removing bitter components in beer brewing industries, removal of pesticides or other contaminants, protection of sensitive motives during processing, e.g., enzymes that preferably needs to be active down-stream of a denaturing process step and where the binding with a specific VAPs would prevent the active site of the enzyme to be denatured, )

(3) personal care products such as shampoos, hair-dying liquids, washing liquids, laundry detergents, gels as applied in different forms such as powders, paste, tablet or liquid form etc. (anti-microbial activity for inhibition of dandruff or other skin-related microbes, anti-microbial activity for toothpastes and mouthwashes, increased specificity for stain-removing enzymes in detergents, stabilizing labile enzymes in soaps or detergents to increase e.g., temperature or pH stability, increased binding activity for hair-dye products, inhibiting enzymes that cause body odours, either in skin applications or in clothing accessories such as shoe-inlays, hygiene tissues)

(4) non-food applications such as printing inks, glues, paints, paper, hygiene tissues etc. (surface-specific inks, glues, paints etcetera for surfaces that are otherwise difficult to print e.g., polyofines -plastic bottles or containers, or for surfaces where highly specific binding is required, e.g., lithographic processes in electronic chip manufacturing, authentication of value papers,)

(5) environmental protection processes such as water purification, bioremediation, clean-up of process waters, concentration of contaminants (removal of microorganisms, viruses, organic pollutants in water purification plants or e.g., green-house water recycling systems, removal of biological hazards from air-ventilation ducts)

(6) animal feed products in dry or wet forms (removal, masking or otherwise inhibiting the effects of anti-nutritional factors that often occur in feed components both for cattle and fish farming, notably protease inhibitors or negative factors such as phytic acid, addition of VAPs as antimicrobial agents to replace current antibiotics with protein-based antibiotics)

Although the preferred embodiments of this patent include industrial processes, the use of VAPs in a manner of affinity chromatography is certainly not limited to these applications. On the “low volume/high value” side of the scale, a variety of applications is feasible for pharmaceutical, diagnostic and research purposes where price is of lesser importance for application, due to the availability of VAPs against ligands that are notoriously difficult to raise antibodies against in classical immune responses. Also the small size and high stability will provide “low volume/high value” applications were VAPs are superior to conventional antibodies or fragments thereof.

(7) pharmaceutical aplications where VAPs can be used as therapeutics themselves, particularly when the core is designed to resemble a natural occurring protein, or to identify and design proper affinity regions and/or core regions for therapeutics.

(8) diagnostic applications where VAPs, as a result of their 3D structure that differ in essential ways from commonly used antibodies or antibody fragments, may detect a different class of molecules. Examples are the detection of infectious prions, where the mutation causing the infectious state is buried inside the native molecule. Conventional antibodies can only discriminate the infectious form under denatured conditions, while the small and exposed AR's of VAPs are able to recognize more inwardly placed peptide sequences.

(9) research applications where VAPs are bound to e.g., plate surfaces or tissues to increase detection levels, localize specific compounds on a fixed surface, fix tracer molecules in position etc. or where selected genes that code for specific VAPs are either transiently, continuously or in a controlled manner expressed by translating the genes in a cellular environment, and where through its targeted expression functional knock-outs of target molecules are formed. For example mimicking a receptor ligand may interfere with normal signal-transduction pathways, or VAPs that function as enzyme inhibitors may interfere with metabolic pathways or metabolic routing.

Diverse as the above examples are, commonalities exist in the ways that VAPs are applied, as is illustrated by the following categories that form a matrix in combination with the applications:

1. affinity chromatography where VAPs are immobilized on an appropriate support e.g., in chromatography columns that can be used in line, in series or in carroussel configurations for fully continuous operation. Also pipes, tubes, in line filters etc. can be lined with immobilized VAPs. The support material on which the VAPs can be immobilized can be chosen to fit the process requirements in terms of compression stability, flow characteristics, chemical inertness, temperature-, pH- and solvent stability etc. Relatively incompressible carriers are preferred, especially silica or rigid inert polymers. These have important advantages for use in industrial-scale affinity chromatography because they can be packed in columns operable at substantially higher pressures than can be applied to softer carrier materials such as agarose. Coupling procedures for binding proteins to such diverse support materials are well-known. After charging the column with the process stream of choice, the bound ligands can be desorbed from the immobilized VAPs through well-known procedures such as changes in pH or salt concentrations after which the VAPs can be regenerated for a new cycle. The high stability of selected VAPs makes them exceptionally suitable for such repeated cycles, thus improving the cost efficiency of such recovery and purification procedures. The principles and versatility of affinity chromatography have been widely described in thousands of different applications.

2. insoluble beads are a different form of affinity chromatography where the support material on which VAPs are immobilized are not fixed in position but are available as beads from for example, silica, metal, magnetic particles, resins and the like. Can be mixed in process streams to bind specific ligands in e.g., fluidized beds or stirred tanks, after which the beads can be separated from the process stream in simple procedures using gravity, magnetism, filters etc.

3. coagulation of target ligands by crosslinking the ligands with VAPs, thereby reducing their solubility and concentrating the ligands through precipitation. For this purpose, VAPs should be bivalent, i.e., at least two AR's must be constructed on either side of the scaffold. The two AR's can have the same molecular target but two different molecular targets are preferred to provide the cross-linking or coagulation effects.

4. masking of specific molecules to protect sensitive motives during processing steps, to increase the stability of the target ligand for adverse pH, temperature or solvent conditions, or to increase the resistance against deteriorating or degrading enzymes. Other functional effects of molecular masking can be the masking of volatile molecules to alter the sensory perception of such molecules. In contrast the slow and conditional release of such molecules from VAPs can be envisaged in more down-stream processing steps, during consumption or digestion or after targeting the VAPs-ligand complex to appropriate sites for biomedical or research applications. Also molecular mimics of volatile compounds using VAPs with specific receptor binding capacity can be used to mask odors from consumer products.

5. coating of insoluble materials with VAPs to provide highly specific surface affinity properties or to bind VAPs or potential fusion products (i.e., products that are chemically bound to the VAPs or, in case of protein, are co-translated along with the VAPs in such manner that the specicifity of the VAPs remains unchanged) to specific surfaces. Examples are the use of VAPs to immobilize specific molecules to e.g., tissues, on plates etc. to increase detection levels, localize specific compounds on a fixed surface, fix tracer molecules in position etc.

Certainly not all natural scaffolds are interesting from a commercial and/or industrial point of view. For example, the stability and sensitivity of the whole protein should meet the requirements that go along with the proposed application. Ligand binding proteins in an acidic environment are not per se useful in high salt or high temperature environments. It is not possible to design one scaffold that has all possible features to function as a one for all scaffold. For example, there are applications that require proteolytic insensitive scaffolds while other applications require specific protease cleavage sites in the scaffold. For these and many other applications it is not possible to design one scaffold that meets all requirements. Therefore we design different scaffolds and adapt these scaffolds to meet the different requirements. As is shown herein, we are able to design and construct such scaffolds with characteristics such as heat stability, a wide pH resistance and ligand binding even in high salt concentrations. Furthermore we are able to adapt the scaffolds to the required characteristics without changing ligand specificity by changing either amino acids in the core or inside or outside oriented amino acids, such as e.g., the introduction or removal of a cysteine bridge or the removal of a potential N-glycosylation site With this in mind, it is possible to design and construct scaffolds that can be used in multiple kinds of ligand binding environments without changing the properties and spatial position of the ligand binding domain. With the above explained MAST technology, selected affinity regions can be swapped from one scaffold to another without losing their ligand specificity, meaning that a once selected affinity can be used in several different applications by just changing the scaffold.

The invention further provides a proteinaceous molecule, method therefore, therewith or use thereof, wherein the proteinaceous molecule comprises a molecule as depicted in table 2, 3, 10, 13 or 16.

### EXAMPLES

### Example 1

Determination of Core Coordinates

Immunoglobulin-like (ig-like) folds are very common throughout nature. Many proteins, especially in the animal kingdom, have a fold region within the protein that belongs to this class. Reviewing the function of the proteins that contain an ig-like fold and reviewing the function of this ig-like fold within that specific protein, it is apparent that most of these domains, if not all, are involved in ligand binding. Some examples of ig-like fold containing proteins are: V-CAM, immunoglobulin heavy chain variable domains, immunoglobulin light chain variable domains, constant regions of immunoglobulins, T-cell receptors, fibronectin, reovirus coat protein, beta-galactosidase, integrins, EPO-receptor, CD58, ribulose carboxylase, desulphoferrodoxine, superoxide likes, biotin decarboxylase and P53 core DNA binding protein. A classification ofmost ig-like folds can be obtained from the SCOP database (Murzin A. G et al, 1995; http://scop.mrc-lmb.cam.ac.uk/scop) and from CATH (Orengo et al, 1997; http://www.biochem.ucl.ac.uk/bsm/cath_new/index.html). SCOP classifies these folds as: all beta proteins, with an immunoglobulin-like beta-sandwich in which the sandwich contains 7 strands in 2 sheets although some members that contain the fold have additional strands. CATH classifies these folds as mainly beta proteins with an architecture like a sandwich in an immunoglobulin-like fold designated with code 2.60.40. In structure database like CE (Shindyalov et al.1998; http://cl.sdsc.edu/ce.htm), VAST (Gibrat et al.,1996; http://www.ncbi.nlm.nih.gov/Structure/VAST/vast.shtml) and FSSP (Holm et al, 1998; http://www.ebi.ac.uk/dali/fssp) similar classifications are used.

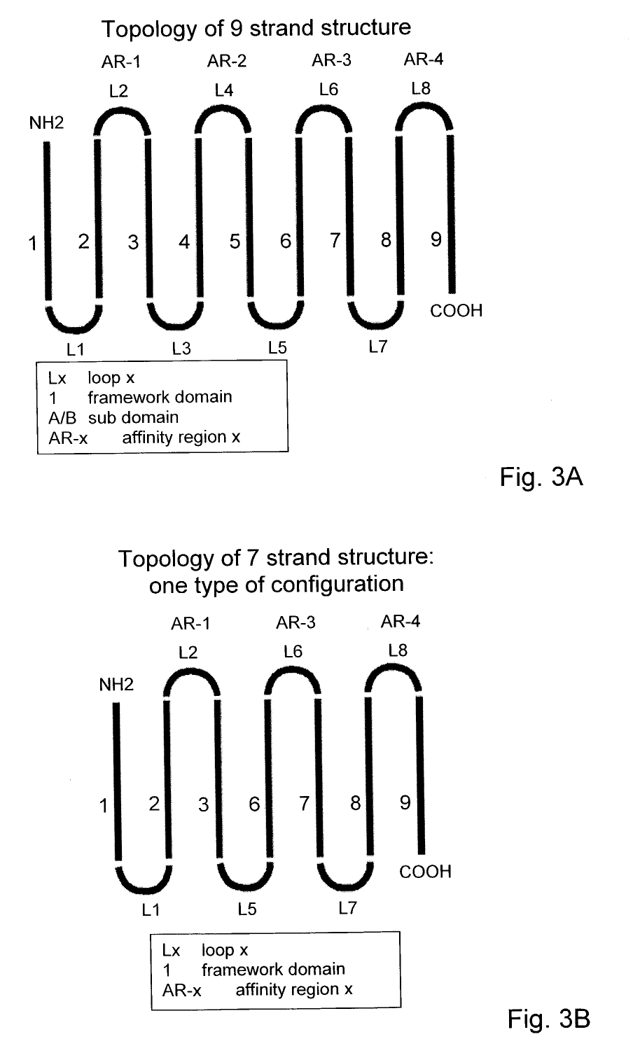

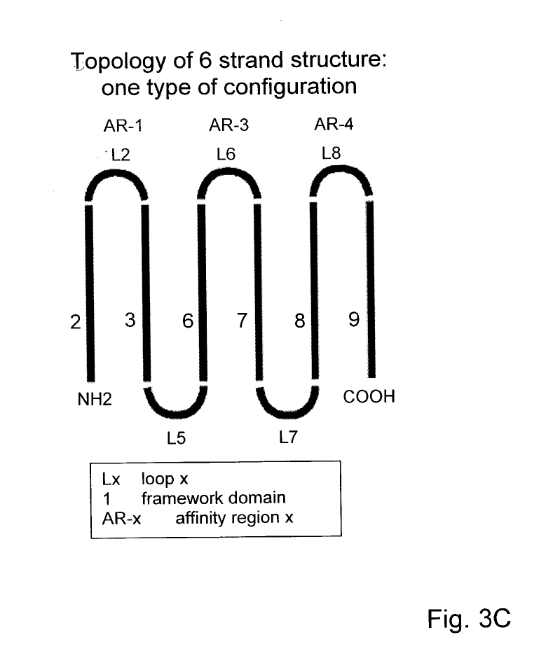

Projection of these folds from different proteins using software of Cn3D (NCBI; http://www.ncbi.nlm.nih.gov/Structure/CN3D/cn3d.shtml), InsightII (MSI; http://www.accelrys.com/insight) and other structure viewers, showed that the ig-like folds have different sub-domains. A schematic projection of the structure is depicted in FIG. 3A. Themost conserved structure was observed in the centre of the folds, named the core. The core structures hardly vary in length and have a relative conserved spatial constrain, but they were found to vary to a large degree in both sequence and amino acid composition. On both sides of the core, extremely variable sub-domains were present that are called connecting loops. These connecting loops can vary in amino acid content, sequence, length and configuration. The core structure is therefore designated as the most important domain within these proteins. The number of beta- elements that form core can vary between 7 and 9 although 6 stranded core structures might also be of importance. The beta-elements are all arranged in two beta-sheets. Each beta-sheet is build of anti-parallel beta-element orientations. The minimum number of beta-elements in one beta-sheet that was observed was 3 elements. The maximum number of beta-element in one sheet that was observed was 5 elements. Higher number of beta-elements might be possible. Connecting loops connect the beta-elements on one side of the barrel. Some connections cross the beta-sheets while others connect beta-elements that are located within one beta-sheet. Especially the loops that are indicated as L2, L4, L6 and L8 are used in nature for ligand binding. The high variety in length, structure, sequences and amino acid compositions of the L1, L3, L5 and L7 loops clearly indicates that these loops can also be used for ligand binding, at least in an artificial format.

Amino acid side chains in the beta-elements that form the actual core that are projected towards the interior of the core and thus fill the space in the centre of the core, can interact with each other via H-bonds, covalent bonds (cysteine bridges) and other forces, to stabilize the fold. Because amino acid composition and sequence of the residues of the beta-element parts that line up the interior were found to be extremely variable it was concluded that many other formats and can also be created.

In order to obtain the basic concept of the structure as a starting point for the design of new types of proteins containing this ig-like fold, projections of domains that contain ig-like folds were used. Insight II, Cn3D and modeller programs were used to determine the minimal elements and lengths. In addition, as amino acid identities were determined as not of any importance, only C-alpha atoms of the structures were projected because these described the minimal features of the folds. Minor differences in spatial positions (coordinates) of these beta elements were allowed. Four examples of such structures containing 9 beta elements were determined and converted into PDB formats (coordinate descriptions; see table 1) but many minor differences within the structure were also assumed to be of importance, as long as the fold according to the definitions of an ig-like fold (see e.g., CATH and SCOP).

These PDB files representing the coordinates of the C-alpha atoms of the core of ig-like folds were used for the development of new 9, 8, 7 and 6 beta-elements containing structures. For 8 stranded structures beta element 1 or 9 can be omitted but also elements 4 or 5 can be omitted. For 7 stranded structures, beta elements 1 and 9 were removed or, preferably, elements 4 and 5 were omitted. The exclusion of elements 4 and 5 is preferred because of spatial constrains (FIG. 3B). Six stranded structures lack preferably element 1, 4 and 5 or 4, 5 and 9 but also other formats were analyzed with Insight and modeller and shown to be reliable enough for engineering purposes (FIG. 3C).

### Example 2

Design of 9 Strands Folds

Protein folding depends on interaction between amino acid backbone atoms and atoms present in the side chains of amino acids. Beta sheets depend on both types of interactions while interactions between two beta sheets, for example in the abovementioned structures, are mainly mediated via amino acid side chain interactions of opposing residues. Spatial constrains, physical and chemical properties of amino acid side chains limit the possibilities for specific structures and folds and thus the types of amino acids that can be used at a certain location in a fold or structure. To obtain amino acid sequences that meet the spatial constrains and properties that fit with the 3D structure of the above described structures (example 1), 3D analysis software (Modeller, Prosa, Insight II, What if and Procheck) was used. Current computer calculation powers and limited model accuracy and algorithm reliabilities limit the number of residues and putative structures that can be calculated and assessed.

To obtain an amino acid sequence that can form a 9 beta strand folds as described above, different levels of testing are required, starting with a C-alpha backbone trace as described in for example PDB file 1. First the interior of the fold needs to be designed and tested. Secondly, beta-element connecting loops need to be attached and calculated. Thirdly exterior amino acids, i.e., amino acids that expose their amino acid side chains to the environment, need to fit in without disturbing the obtained putative fold. In addition, the exterior amino acid side chains should preferably result in a soluble product. In the fourth and last phase, the total model is recalculated for accidentally introduced spatial conflicts. Amino acid residues that provoked incompatibilities are exchanged by an amino acid that exhibits amore accurate and reliable fit.

In the first phase, amino acid sequences aligning the interior of correctly folded double beta-sheet structures that meet criteria as described above and also in example 1, were obtained by submitting PDB files for structural alignments in e.g., VAST (http://www.ncbi.nlm.nih.gov/Structure/VAST/vast.shtml). The submission of the PDB files as depicted in PDB file 1 already resulted in thousands of hits. The majority of these proteins were proteins that contained at least one domain that would be classified according to SCOP or CATH (see above) as folds meant here.

Several unique sequences aligning the interior of the submitted structure were used for the generation of product examples. Interesting sequences from this structural alignment experiment were selected on criteria of classification, root mean square deviations (RMSD-value), VAST-score values (higher values represent more accurate fit), sequence identities, origin of species and proposed biological function of the hits. Structures as fibronectin-like protein, antibody related proteins, cell adhesion molecules, virus core proteins, and many others. The structures that are represented by the C-alpha backbones are called the core structures.

In the second phase, loops were attached to obtained products. Although several analysis methods can be applied that resolve the structure of the end products, the most challenging feature would be the presentation of affinity regions on core sequences that have full functional ligand binding properties. In order to test the functionality of the end products, affinity loops that recognize known ligands can be transplanted on the core structure. Because anti-chicken lysozyme (structure known as 1MEL) is well documented, and the features of these affinity regions (called CDR's in antibodies) are well described, these loops were inserted at the correct position on core sequences obtained via the method described in the first phase. Correct positions were determined via structural alignments, i.e., overlap projections of the already obtained folds with the file that describes the 3D structure of 1MEL (PDB file; example). Similar projections and subsequent loop transplantations were carried out for the bovine RNase A binding affinity region that were extracted from the structure described by 1BZQ (PDB). The transplanted affinity loops connect one end of the beta elements with one other. Affinity region 1 connects beta-element 2 with 3 (L2), AR2 connects beta element 4 and 5 (L4), AR3 connects beta elements 6 and 7 (L6) and AR4 connects beta elements 8 and 9 (L8). The other end of each of the beta elements was connected by loops that connect element 1 with 2 (L1), 3 with 4 (L3), 5 with 6 (L5) and 7 with 8 (L7) respectively (see schematic projection in FIG. 3A). Of course all kinds of loops can be used to connect the beta elements. Sources of loop sequences and loop lengths encompass for example loops obtained via loop modeling (software) and from available data from natural occurring loops that have been described in the indicated classes of for example SCOP and CATH. C-alpha backbones of loops representing loops 1 (L1), 3 (L3), 5 (L5) and 7 (L7; FIG. 3A) were selected from structures like for example 1NEU, 1EPF-B, 1QHP-A, 1CWV-A, 1EJ6-A, 1E50-C, 1MEL, 1BZQ and 1F2X, but many others could have been used with similar results. 3D-aligments of the core structures obtained in the first phase as described above, together with loop positions obtained from structural information that is present in the PDB files of the example structures 1EPF, 1NEU, 1CWV, 1F2X, 1QHP, 1E50 and 1EJ6 were realized using powerful computers and Cn3D,modeller and/or Insight software of. Corresponding loops were inserted at the correct position in the first phase models. Loops did not have to fit exactly on to the core because a certain degree of energy and/or spatial freedom can be present. The type of amino acids that actually will form the loops and the position of these amino acids within the loop determine this energy freedom of the loops. Loops from different sources can be used to shuffle loop regions. This feature enables new features in the future protein because different loops have different properties, like physical, chemical, expressional, post translational modifications, etc. Similarly, structures that contain less loops due to reduced numbers of beta elements can be converted into proteins with 9 beta elements and a compatible number of loops. Here it is demonstrated that the C-alpha trace backbones of the loops of 7 stranded proteins like for example 1EPF, 1QHP, 1E50 and 1CWV could be used as templates for 9 stranded core templates. The additional loop (L3) was in this case retrieved from the 9 stranded template 1F2X but any other loops that were reliable according to assessment analysis could also have been used. The nature of the amino acids side chains that are pointing to the interior of the protein structure was restricted and thus determined by spatial constrains. Therefore several but limited configurations were possible according to 3D-structure projections using the modeling software.

In the third phase, all possible identifies of amino acid side chains that are exposed to the exterior, i.e., side chains that stick out of the structure into the environment, were calculated for each location individually. For most applications, it is preferred to use proteins that have very good solubility, and therefore amino acids were chosen that are non-hydrophobic. Such amino acids are for example D, E, N, Q, R, S and T. Methionine was preferably omitted because the codon belonging to methionine (ATG) can results in alternative proteins products due to aberrant translational starts. Also, cysteine residues were omitted because free cysteines can lead to cysteine-cysteine bonds. Thus, free cysteines can result in undesired covalent protein-protein interactions that contain free cysteines. Glycine residues can be introduced at locations that have extreme spatial constrains. These residues do not have side chains and are thus more or less neutral in activity. However, the extreme flexibility and lack of interactive side chains of glycine residues can lead to destabilization and therefore glycine residues were not commonly used.

In the fourth phase, the models were assessed using modeller. Modeller was programmed to accept cysteine-cysteine bridges when appropriate. Next all predicted protein structures were assessed with Prosall (http://www.came.sbg.ac.at/Services/prosa.html), Procheck and What if (http://www.cmbi.kun.nl/What if). Prosall zp-comb scores of less then -4.71 were assumed to indicate protein sequences that might fold in vivo into the desired beta motif. The seven protein sequences depicted in table 1 represent a collection of acceptable solutions meeting all criteria mentioned above. Procheck and What if assessments also indicated that these sequences might fit into the models and thus as being reliable (e.g., pG values larger than 0.80; Sanchez et al., 1998)

### Example 3

Assembly of Synthetic Scaffolds Synthetic VAPs were designed on basis of their, predicted, three dimensional structure. The amino acid sequence (Table 3) was back translated into DNA sequence (Table 4) using the preferred codon usage for enteric bacterial gene expression (Informax Vector Nti). The obtained DNA sequence was checked for undesired restriction sites that could interfere with future cloning steps. Such sites were removed by changing the DNA sequence without changing the amino acid codons. Next the DNA sequence was adapted to create a NdeI site at the 5′ end to introduce the ATG start codon and at the 3end a SfiI site, both required for unidirectional cloning purposes. PCR assembly consists of four steps: oligo primer design (ordered at Operon's), gene assembly, gene amplification, and cloning. The scaffolds were assembled in the following manner: first both plus and minus strands of the DNA sequence were divided into oligonucleotide primers of approximately 35 bp and the oligonucleotide primer pairs that code for opposite strands were designed in such a way that they have complementary overlaps of approximately 16-17 bases. Second, all oligonucleotide primers for each synthetic scaffold were mixed in equimolar amounts, 100 pmol of this primer mix was used in a PCR assembly reaction using 1 Unit Taq polymerase (Roche), 1×PCR buffer+mgCl₂ (Roche) and 0.1 mM dNTP (Roche) in a final volume of 50 μl, 35 cycles of; 30 sec. 92° C., 30 sec. 50° C., and 30 sec. 72° C. Third, 5 μl of PCR assembly product was used in a standard PCR amplification reaction using, both outside primers of the synthetic scaffold, 1 Unit Taq polymerase, 1×PCR buffer+mgCl₂, and 0.1mM dNTP in a final volume of 50 μl, 25 cycles; 30 sec. 92° C., 30 sec. 55° C., 1 min. 72° C. Fourth, PCR products were analyzed by agaros gel electrophoresis, PCR products of the correct size were digested with NdeI and SfiI and ligated into vector pCM126 linearized with NdeI and SfiI. Ligation products were transformed into TOP10 competent cells (In Vitrogen) grown overnight at 37° C. on 2xTY plates containing 100 microgram/ml ampicillin and 2% glucose. Single colonies were grown in liquid medium containing 100 μg ampicillin, plasmid DNA was isolated and used for sequence analysis.

### Example 4

Expression Vector CM126 Construction

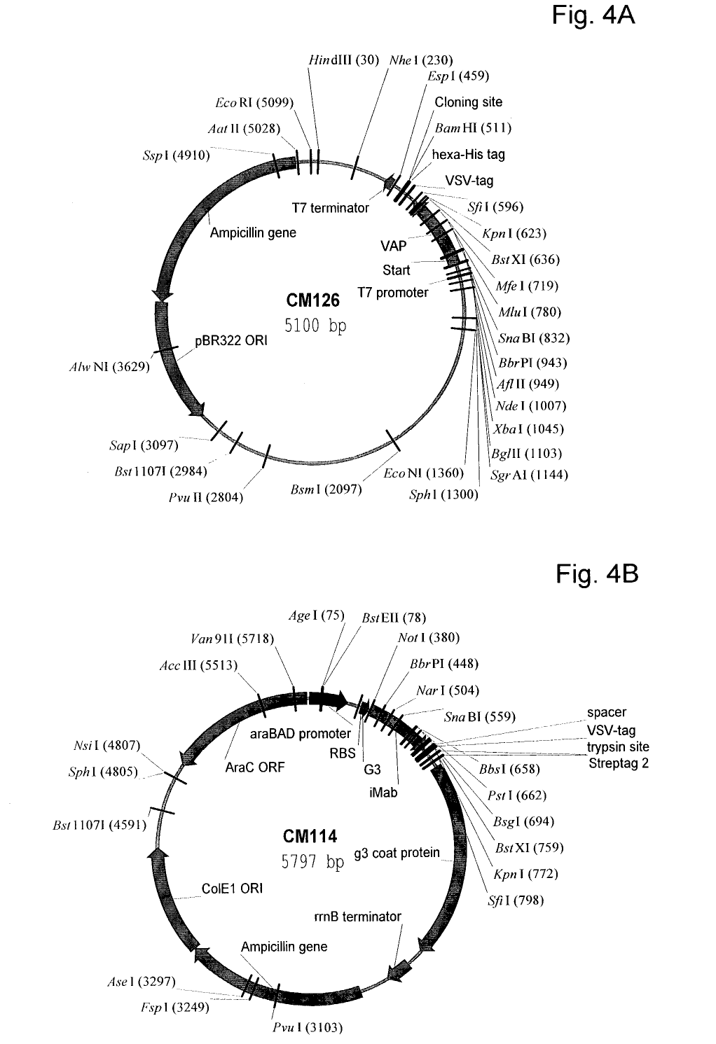

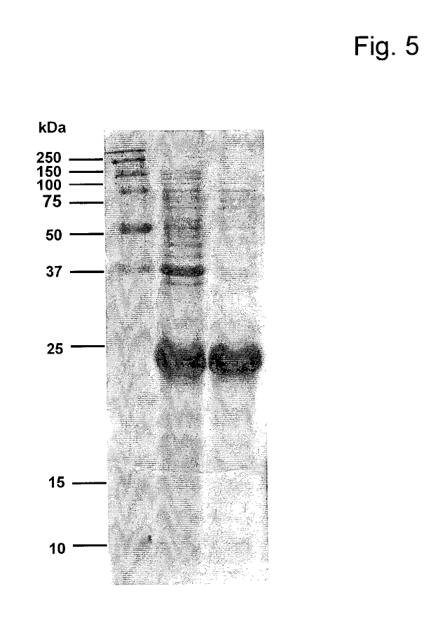

A vector for efficient protein expression (CM126; see FIG. 4A) based on pET-12a (Novagen) was constructed. A dummy VAP, iMab100, including convenient restriction sites, linker, VSV-tag, 6 times His-tag and stop codon was inserted (see table 4, 3). As a result, the signal peptide OmpT was omitted from pET-12a. iMab100 was PCR amplified using forward primer 129 (see Table 5) that contains a 5′ NdeI overhanging sequence and a very long reverse oligonucleotide primer 306 (see Table 5) containing all linkers and tag sequences and a BamHI overhanging sequence. After amplification, the PCR product and pET-12a were digested with NdeI and BamHI. After gel purification products were purified via the Qiagen gel-elution system according to manufacturer's procedures. The vector and PCR fragment were ligated and transformed by electroporation in E. coli TOP10 cells. Correct clones were selected and verified for their sequence by sequencing. This vector including the dummy VAP acted as the basic vector for expression analysis of other VAPs. Insertion of other VAPs was performed by amplification with primers 129 and 51 (see Table 5), digestion with NdeI and SfiI and ligation into NdeI and SfiI digested CM126.

### Example 5

Expression of iMab100

E. coli BL21 (DE3) (Novagen) was transformed with expression vector CM 126-iMab100. Cells were grown in 250 ml shaker flasks containing 50 ml 2*TY medium (16 g/l tryptone, 10 g/l yeast extract, 5 g/l NaCl (Merck)) supplemented with ampicillin (200 microgram/ml) and agitated at 30° C. Isopropylthio-β-galactoside (IPTG) was added at a final concentration of 0.2 mM to initiate protein expression when OD (600 nm) reached one. The cells were harvested 4 hours after the addition of IPTG, centrifuged (4000 g, 15 min., 4° C.) and pellets were stored at −20° C. until used.

Protein expression was analyzed by Sodium Dodecyl Sulphate PolyAcrylamide Gel Electrophoresis (SDS-PAGE). This is demonstrated in FIG. X lane 2 for E. coli BL21(CM 126-iMab100) expressing iMAb100.

### Example 6

Purification of iMab100 Proteins from Inclusion Bodies using Heat.

IMab100 was expressed in E. coli BL21 (CM126-iMab100) as described in example 5.most of the expressed iMab100 was deposited in inclusion bodies. This is demonstrated in FIG. X lane 3, which represents soluble proteins of E. coli BL21 (CM126) after lysis (French press) and subsequent centrifugation (12.000 g, 15 min). Inclusion bodies were purified as follows. Cell pellets (from a 50 ml culture) were resuspended in 5 ml PBS pH 8 up to 20 g cdw/1 and lysed by 2 passages through a cold French pressure cell (Sim-Aminco). Inclusion bodies were collected by centrifugation (12.000 g, 15 min) and resuspended in PBS containing 1% Tween-20 (ICN) in order to solubilize and remove membrane-bound proteins. After centrifugation (12.000 g, 15 min), pellet (containing inclusion bodies) was washed 2 times with PBS. The isolated inclusion bodies were resuspended in PBS pH 8+1% Tween-20 and incubated at 60° C. for 10 minutes. This resulted in nearly complete solubilization of iMab100 as is demonstrated in FIG. 5. Lane 1 represents isolated inclusion bodies of iMab100. Lane 2 represents solubilized iMab100 after incubation of the isolated inclusion bodies in PBS pH 8+1% Tween-20 at 60° C. for 10 minutes.

The supernatant was loaded on a Nickel-Nitrilotriacetic acid (Ni-NTA) superflow column and purified according to a standard protocol as described by Qiagen (The QIA expressionist™, fifth edition, 2001). The binding of the thus purified iMab100 to chicken lysozyme was analyzed by ELISA (according to Example 8) and is summarized in Table 6.

### Example 7

Purification of iMab100 Proteins from Inclusion Bodies using Urea Andmatrix Assisted Refolding

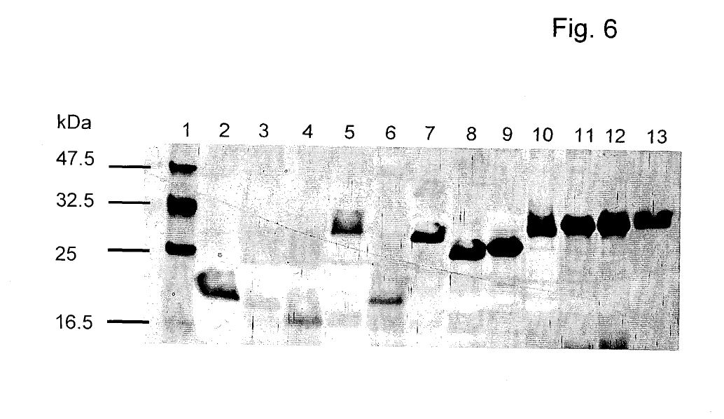

Alternatively, iMab100 was solubilized from inclusion bodies using 8 m urea and purified into an active form bymatrix assisted refolding. Inclusion bodies were prepared as described in example 6 and solubilized in 1 ml PBS pH 8+8 m urea. The solubilized proteins were clarified from insolublematerial by centrifugation (12.000 g, 30 min.) and subsequently loaded on a Ni-NTA super-flow column (Qiagen) equilibrated with PBS pH 8+8 M urea. Aspecific proteins were released by washing the column with 4 volumes PBS pH 6.2+8 M urea. The bound His-tagged iMab100 was allowed to refold on the column by a stepwise reduction of the urea concentration in PBS pH 8 at room temperature. The column was washed with 2 volumes of PBS+4 M urea, followed by 2 volumes of PBS+2 M urea, 2 volumes of PBS+1 M urea and 2 volumes of PBS without urea. IMab100 was eluted with PBS pH 8 containing 250 mM imidazole. The released iMab100 was dialyzed overnight against PBS pH 8 (4° C.), concentrated by freeze drying and characterized for binding and structure measurements. The purified fraction of iMab100 was analyzed by SDS-PAGE as is demonstrated in FIG. 6. lane 13.

### Example 8

Specific Binding of iMab100 Proteins to Chicken Lysozyme (ELISA)

Binding of iMab proteins to targetmolecules was detected using an Enzyme Linked Immnuno Sorption Assay (ELISA). ELISA was performed by coating wells of microtiter plates (Nunc) with the desired antigen (such as chicken lysozyme) and blocked with an appropriate blocking agent such as 3% skimmilk powder solution (ELK). Purified iMab proteins or purified phages (10⁸-10⁹) originating from a single colony were added to each well and incubated for 1 hour at room temperature. Plates were excessively washed with PBS containing 0.1% Tween-20 using a plate washer (Bio-Tek Instruments). Bound iMab proteins or phages were detected by the standard ELISA protocol using anti-VSV-hrp conjugate (Roche) or anti-M13-hrp conjugate (Pharmacia), respectively. Colorimetric assays were performed using Turbo-TMB (3, 3′, 5, 5′—tetramethylbenzidine, Pierce) as a substrate.

Binding of iMab100 to chicken lysozyme was assayed as follows. Purified iMab100 (˜50 ng) in 100 μl was added to a microtiter plate well coated with either ELK (control) or lysozyme (+ELK as a blocking agent) and incubated for 1 hour at room temperature on a table shaker (300 rpm). Themicrotiter plate was excessively washed with PBS (3 times), PBS+0.1% Tween-20 (3 times) and PBS (3 times). Bound iMab100 was detected by incubating the wells with 100 μl ELK containing anti-VSV-HRP conjugate (Roche) for 1 hour at room temperature.

After excessive washing using PBS (3 times), PBS+0.1% Tween-20 (3 times) and PBS (3 times), wells were incubated with 100 μl Turbo-TMB for 5minutes. Reaction was stopped with 100 μl 2M H₂SO₄ and absorbtion was read at 450 nm using amicrotiter plate reader (Biorad).

Purified iMab100 which has been prepared as described in Example 6 and Example 7 appeared to bind strongly and specifically to chicken lysozyme which is demonstrated in Table 6.

### Example 9

Size Exclusion Chromatography

IMab100 was purified as described in Example 7.

The purified iMab100 was analyzed for molecular weight distribution using a Shodex 803 column with 40% acetonitrile, 60% milliQ and 0.1% TFA as mobile phase. 90% of the protein eluted at a retention time of 14.7 minutes corresponding to a molecular weight of 21.5 kD. This is in close agreement with the computer calculated molecular weight (19.5 kD) and indicates that most of the protein is present in the monomeric form.

### Example 10

iMab100 Stability at 95° C. over Time

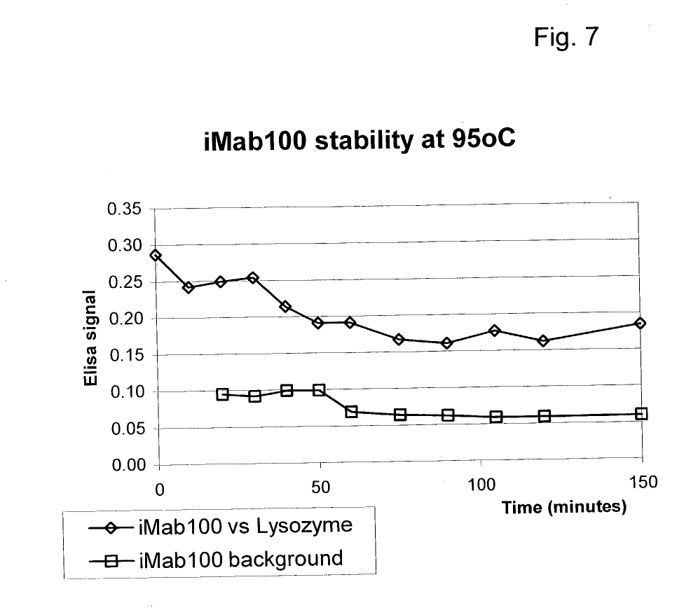

iMab100 stability was determined at 95° C. by ELISA. 10 microgram/milliliter iMab100 was heated to 95° C. for 10 minutes to 2.5 hours, unheated iMab was used as input control. After heating, samples were placed at 20° C. and kept there until assayed. Lysozyme binding of these samples was tested by ELISA measurements using 1:2000 in PBS diluted anti-VSV-hrp (Roche). TMB-ultra (Pierce) was used as a substrate for hrp enzyme levels (FIG. 7). iMab100 was very stable at high temperatures. A very slow decrease in activity was detected.

### Example 11

iMab100 Stability over Time at 20° C.

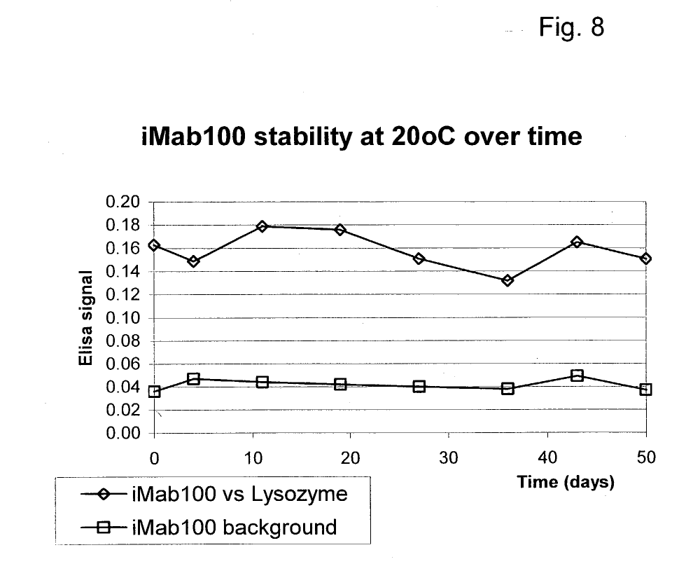

iMab100 stability was determined over a period of 50 days at 20° C. iMab100 (0.1 milligram/milliliter) was placed at 20° C. Every 7 days a sample was taken and every sample was stored at −20° C. for at least 2 hours to prevent breakdown and freeze the experimental condition. Samples were diluted 200 times in PBS. Lysozyme binding of these samples was tested by ELISA measurements using 1:2000 in PBS diluted anti-VSV-hrp (Roche). TMB-ultra (Pierce) was used as a substrate for hrp enzyme levels (FIG. 8). iMab100 was very stable at room temperature. Activity of iMab100 hardly decreased over time, and thus it can be concluded that the iMab scaffold and its affinity regions are extremely stable.

### Example 12

iMab100 size determination, resistance against pH 4.8 environment, testing by gel and Purified iMab100 (as described in Example 6) was brought to pH 4.8 using potassium acetate (final concentration of 50 mM) which resulted in precipitation of the protein. The precipitate was collected by centrifugation (12000 g, 30 minutes), re-dissolved in PBS pH 7.5 and subsequently filtered through a 0.45 micrometer filter to remove residual precipitates.

The samples before and after pH shock were analyzed by SDS-PAGE, western blotting and characterized for binding using ELISA (Example 8).

It was demonstrated that all iMab100 was precipitated at pH 4.8 and could also be completely recovered after re-dissolving in PBS pH 7.5 and filtering. ELISA measurements demonstrated that precipitation and subsequent resolubilization did not result in a loss of activity (Table 7). It was confirmed that the VSV-tag is not lost during purification and precipitation and that no degradation products are formed.

### Example 13

Structural Analysis of Scaffolds

The structure of iMab100 was analyzed and compared with another structure using a circular dichroism polarimeter (CD). As a reference, a naturally occurring 9 beta strand containing Vhh molecule, Vhh10-2/271102 (a kind gift of M. Kwaaitaal, Wageningen University), was measured. Both proteins have tags attached to the C-terminal end. The amino acid sequence and length of these tags are identical. The only structural differences between these two proteins are present in the CDR3 (Vhh) corresponding affinity loop 4 (iMab100).

System settings were: sensitivity=standard (100 mdeg); start=260 nm; end=205 nm; interval=0.1 nm; delay=1 sec.; speed=50 nm/min; accumulation=10.

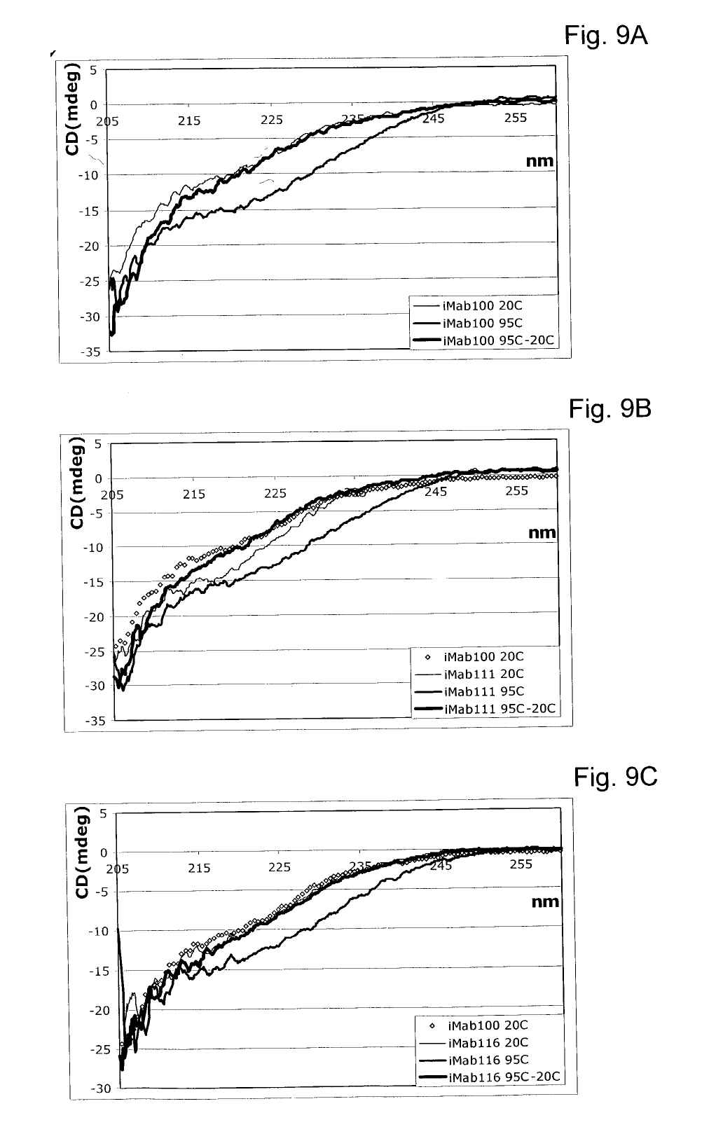

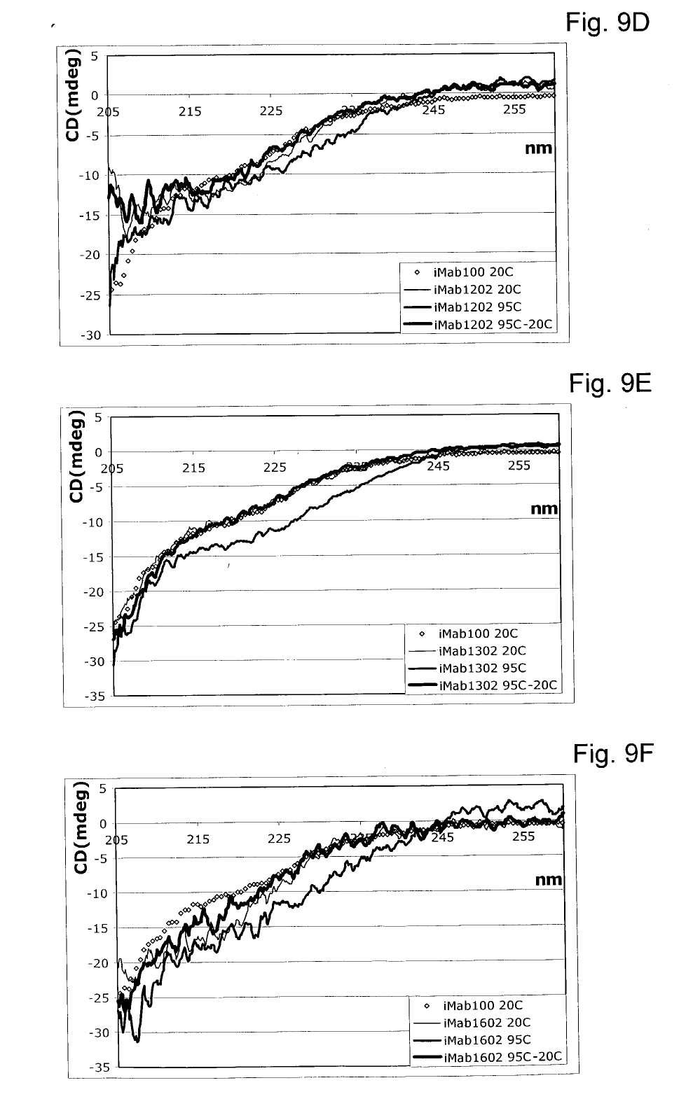

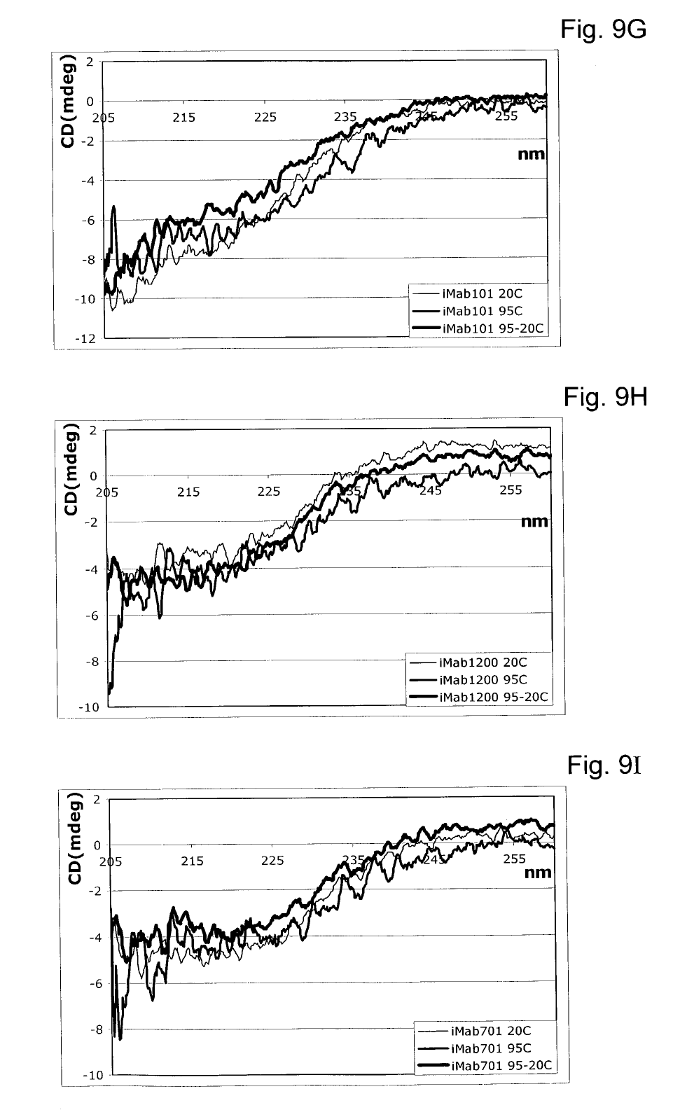

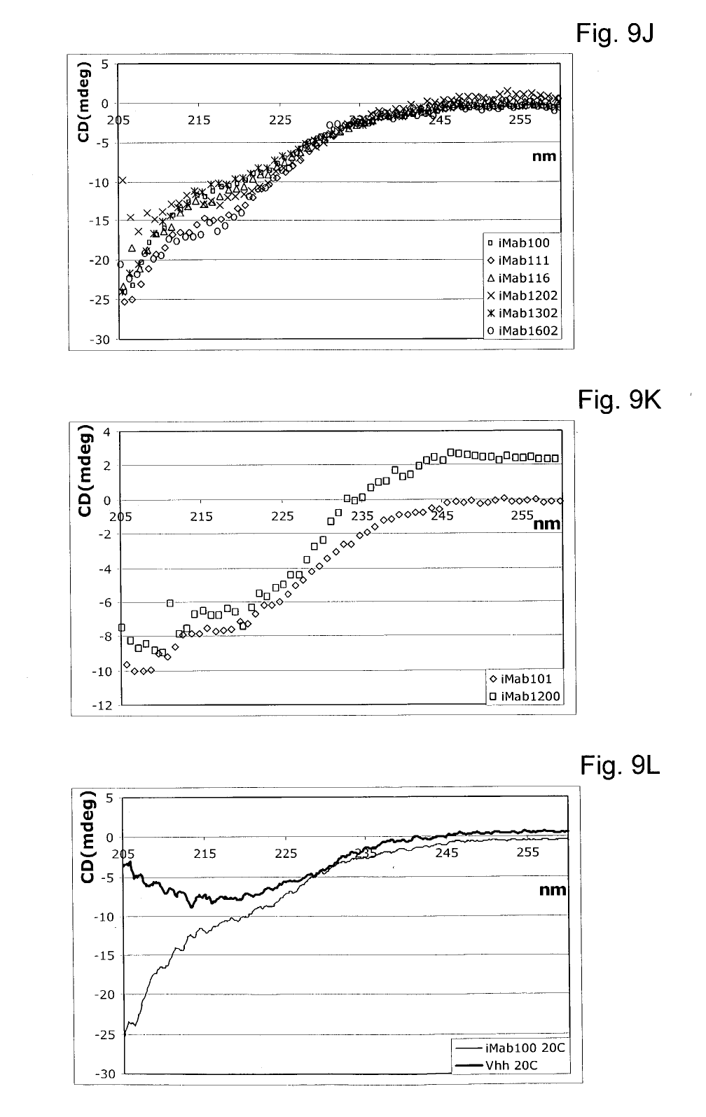

iMab100 and Vhh10-2/271102 were prepared with a purity of 98% in PBS pH 7.5 and OD₂₈₀ ≈1.0. Sample was loaded in a 0.1 cm quartz cuvette and the CD spectrum measured with a computer controlled JASCO Corporation J-715 spectropolarimeter software (Spectramanager version 1.53.00, JASCO Corporation). Baseline corrections were obtained by meausring the spectrum of PBS. The obtained PBS signal was substracted from all measurements to correct for solvent and salt effects. An initial measurement with each sample was done to determine the maximum signal. If required, the sample was diluted with 1 times PBS fro optimal resolution of the photomultiplier signal. A solution in PBS of RNase A was used to verify the CD apparatus. The observed spectrum of RNase A was completely different if compared with iMab100 and the Vhh spectrum. FIG. 9L represents the CD spectrum of iMab100 and the Vhh proteins in far UV (205-260nm). Large part of the spectral patterns were identical. Spectral difference were mainly observed at wavelengths below 220 nm. The observed differences of the spectra are probably due to differences in CDR3/AR4 structural differences. The structure of AR4 in iMab100, which was retrieved from 1MEL, can be classified as random coil-like. Also, AR4 present in iMab100 is about 10 amino acids longer than the CDR3 of the Vhh protein.

The temperature stability of the iMab100 protein was determined in a similar way using the CD-meter except that the temperature at which the measurements were performed were adjusted. In addition to measurements at room temperature, folding and refolding was assayed at 20, 50, 80 (not shown) and 95 degrees Celsius. Fresh iMab100 protein solution in PBS diluted was first measured at 20 degrees Celsius. Next, spectra at increasing temperatures were determined and lastly, the 20 degrees Celsius spectrum was re-measured. Baseline corrections were applied with the spectrum of PBS (FIG. 9A). The results clearly show a gradual increase in ellipticity at increasing temperatures. The re-appearance of the 20 degrees Celsius spectrum after heating strongly indicates complete refolding of the scaffold. This conclusion was also substantiated by subsequent lysozyme binding capacity detection of the samples by ELISA (data not shown).

### Example 14

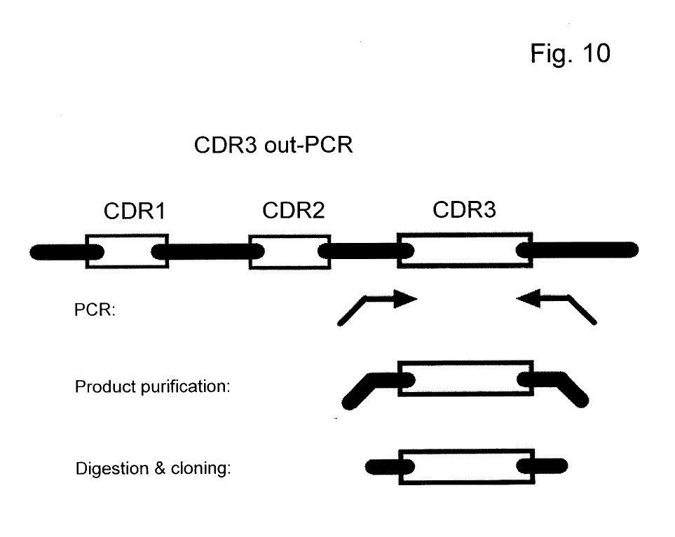

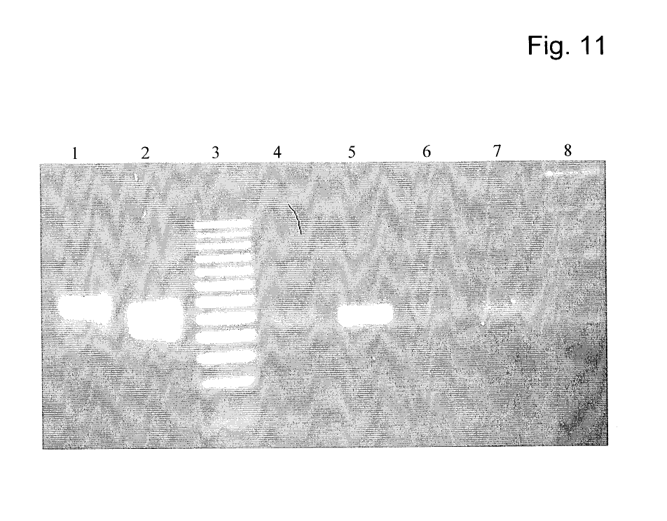

E. coli BL21 (DE3) (Novagen) was transformed with expression vector CM126 containing various VAP inserts for iMab1302, iMab1602, iMab1202 and iMab122 all containing 9 β-strands. Growth and expression was similar as described in Example 5. All 9-stranded iMab proteins were purified by matrix assisted refolding similar as is described in example 7. The purified fractions of iMab1302, iMab1602, iMab1202 and iMab122 were analyzed by SDS-PAGE as is demonstrated in FIG. 10 lanes10, 9, 8 and 7 respectively.

### Example 15

Specific Binding of Various 9 Stranded iMab Proteins to Chicken Lysozyme (ELISA)

Purified iMab1302(˜50 ng), iMab1602(˜50 ng), iMab1202(˜50 ng) and iMab122 (˜50 ng) were analyzed for binding to either ELK (control) or lysozyme (+ELK as a blocking agent) similar as is described in Example 8. ELISA confirmed specific binding of purified iMab1302, iMab1602, iMab1202 and iMab122 to chicken lysozyme as is demonstrated in Table 6

### Example 16

CD Spectra of Various 9 Stranded iMab

iMab100, iMab1202, Imab1302 and iMab1602were purified as described in example 14 and analyzed for CD spectra as described in example 13. The spectra of iMab1202, iMab1302 and iMab1602 were measured at 20° C., 95° C. and back at 20° C. to test scaffold stability and refolding characteristics. The corresponding spectra are demonstrated in FIG. 9D, 9E and 9F respectively. The spectra measured at 20° C. were compared with the spectrum of iMab100at 20° C. to determine the degree of similarity of the secondary structure (see, FIG. 9J). It can be concluded that all different 9 strand scaffolds behave identical. This indicates that the basic structure of these scaffolds is identical. The data obtained after succesive 20-95-20 degrees Celsius treatments clearly show that all scaffolds return to their original conformation.

### Example 17

Design of 7 Stranded ig-Like Folds

The procedure as described in Example 2 was used for the development of sequences that contain an ig-like fold consisting of 7 beta-elements in the core and 3+3 connecting loops. The procedure involved 4 phases through which the development of the new sequences was guided, identical as the process as described in example 2. In phase 1, the coordinates of C-alpha atoms as indicated in PDB table 1 for 9 stranded core structures were adapted. C-alpha atoms representing beta elements 4 and 5 were removed from the PDB files, resulting in a seven-stranded example of the core (PDB table 8). Amino acid side chains that line up with the interior of the beta-sheets were obtained and inserted as described in detail in example 2. In the second phase connecting loops were added. On one site beta-elements were connected with one other by affinity region retrieved from anti-chicken lysozyme binding region obtained from the structure 1MEL or the bovine RNase A binding regions of 1BZQ (L2, L6 and L8). On the other end of the structure, beta-elements were connected with C-alpha backbone trace loops obtained from several different origins (1E50, 1CWV, 1QHP, 1NEU, 1EPF, 1F2x or 1EJ6). The procedure for the attachment and fit of the loops is described in detail in example 2. In the third phase, amino acid side chains that determine the solubility of the proteins located in the core and loops 1, 3, 7 were determined as described in example 2. In the last phase, the models were build using Insight. Insight was programmed to accept cysteine-cysteine bridges when appropriate. Next all predicted protein structures build with Insight were assessed with Prosall, Procheck and WHAT IF. Prosall zp-comb scores of less then −4.71 were assumed to indicate protein sequences that might fold in vivo into the desired ig-like beta motif fold (table 9). A number of example sequences depicted in table 10 represent a collection that appeared to be reliable. Procheck and What if assessments also indicated that these sequences might fit into themodels and thus as being reliable (e.g., pG values larger than 0.80; Sanchez et al., 1998).

### Example 18

E. coli BL21 (DE3) (Novagen) was transformed with expression vector CM126 containing various VAP inserts for iMab1300, iMab1200, iMab101 and iMab900all containing 7 beta-strands. Growth and expression was similar as described in Example 5. All 7-strand iMabs were purified bymatrix assisted refolding similar as is described in Example 7. The purified fractions of iMab101, iMab1300, iMab1200 and iMab900 were analyzed by SDS-PAGE as is demonstrated in FIG. 10 lanes 2, 3, 5 and 6 respectively.

### Example 19

Purified iMab1300 (˜50 ng), iMab1200 (˜5 ng), iMab101 (˜20 ng) and iMab900 (˜10 ng) were analyzed for binding to either ELK (control) or lysozyme (+ELK as a blocking agent) similar as is described in Example 8. ELISA confirmed specific binding of purified iMab1300, iMab1200, iMab101 and iMab900 to chicken lysozyme as is demonstrated in Table 6.

### Example 20

CD Spectra of Various 7 Stranded iMab Proteins

IMab1200 and iMab101 were purified as described in Example 18 and analyzed for CD spectra as described in Example 13. The spectra of iMab1200 and iMab101 were measured at 20° C., 95° C. and back at 20° C. to test scaffold stability and refolding characteristics. The corresponding spectra are demonstrated in FIGS. 9H and 9G respectively. The spectra of iMab1200 and iMab101 measured at 20° C. were compared with each other to determine the degree of similarity of the secondary structure (see FIG. 9K). It can be concluded that the different 7 strand scaffolds behave identical. This indicates that the basic structure of these scaffolds is identical. Even more, as the obtained signals form the 9 stranded scaffolds (Example 16) are similar to the signals observed for the 7 strands as presented here, it can also be concluded that the both types of scaffolds have an similar conformations. The data obtained after successive 20-95-20 degrees Celsius treatments clearly show that all scaffolds stay in their original conformation.

### Example 21

Design of 6 Stranded ig-like Folds

The procedure as described in Examples 2 and 3 was used for the development of sequences that contain an ig-like fold consisting of six beta-elements in the core and 3+3 connecting loops. The procedure involved 4 phases through which the development of the new sequences was guided, identical as the process as described in Examples 2 and 3. In phase one, the coordinates of C-alpha atoms as indicated in PDB table 1 for 9 stranded core structures were adapted. C-alpha atoms representing beta elements 1, 4 and 5 were removed from the PDB files, resulting in a six-stranded example of the core (Table 11). Amino acid side chains that line up with the interior of the beta-sheets were obtained and inserted as described in detail in Examples 2 and 3. In the second phase, connecting loops were added. On one site beta-elements were connected with one other by affinity region retrieved from anti-chicken lysozyme binding region obtained from the structure 1MEL or the bovine RNase A binding regions of 1BZQ (L2, L6 and L8). On the other end of the structure, beta-elements were connected with C-alpha backbone trace loops obtained from several different origins (1E50, 1CWV, 1QHP, 1NEU, 1EPF, 1F2x or 1EJ6). The procedure for the attachment and fit of the loops is described in detail in example 2 and 3. In the third phase, amino acid side chains that determine the solubility of the proteins located in the core and loops L1, L3, L7 were determined as described in example 2 and 3. In the last phase, the models were assessed using modeller. Modeller was programmed to accept cysteine-cysteine bridges when appropriate. Next all predicted protein structures were assessed with Prosall, Procheck and WHAT IF. Prosall zp-comb scores were determined (Table 12) to indicate if the created protein sequences might fold in vivo into the desired ig-like beta motif fold. Procheck and What if assessments were applied to check whether sequences might fit into the models (Table 13).

### Example 22

Purification of 6 Stranded iMab Proteins

E. coli BL21 (DE3) (Novagen) was transformed with expression vector CM126 containing an VAP insert for iMab701 containing 6 beta-strands. Growth and expression was similar as described in Example 5. The iMab701 proteins were purified by matrix assisted refolding similar as is described in Example 7. The purified fraction of iMab701 was analyzed by SDS-PAGE as is demonstrated in FIG. 6 lane 4.

### Example 23

Specific Binding of 6 Stranded iMab Proteins to Chicken Lysozyme (ELISA)

Purified iMab701 (˜10 ng) was analyzed for binding to either ELK (control) and lysozyme (+ELK as a blocking agent) similar as is described in Example 8. ELISA confirmed specific binding of purified iMab701 to chicken lysozyme as is demonstrated in Table 6.

### Example 24

CD Spectra of a 6 Stranded iMab Proteins

IMab701 was purified as described in Example 22 and analyzed for CD spectra as described in example 13. The spectra of iMab701 was measured at 20° C., 95° C. and again at 20° C. to test scaffold stability and refolding characteristics. The corresponding spectra are demonstrated in FIG. 9I. It can be concluded that the 6 strand scaffold behaves identical to the 7 strand scaffolds as described in example 20. This indicates that the basic structure of this scaffold is identical to the structure of the 7 strand containing scaffolds. Even more, as the obtained signals form the 9 stranded scaffolds (example 16) are similar to the signals observed for this 6 strand scaffold as presented here, it can also be concluded that the both types of scaffolds have an similar conformations. The data obtained after successive 20-95-20 degrees Celsius treatments clearly show that all scaffolds stay in their original conformation.

### Example 25

Design of a Minimal Primary Scaffold

A minimal scaffold is designed according to the requirements and features as described in example 1. However now only four and five beta-elements are used in the scaffold (see FIG. 1). In the case of 5 beta-elements amino acids side chains of beta-elements 2, 3, 6, 7 and 8 that are forming the mantle of the new scaffold need to be adjusted for a watery environment. The immunoglobulin killer receptor 2d12 (VAST code 2DLI) is used as a template for comparative modeling to design a new small scaffold consisting of 5 beta-elements.

### Example 26

Procedure for Exchanging Surface Residues: Lysine Replacements

Lysine residues contain chemical active amino-groups that are convenient in for example covalent coupling procedures of VAPs. Covalent coupling can be used for immobilization of proteins on surfaces or irreversible coupling of other molecules to the target.

The spatial position of lysine residues within the VAP determines the positioning of the VAP on the surface after immobilization. Wrong positioning can easily happen with odd located lysine residues exposed on the surface of VAPs. Therefore, it may be required for some VAP structures to remove lysine residues from certain locations, especially from those locations that can result in diminished availability of affinity regions.

As an example of the exchange strategy for residues that are located on the outer surface, iMab100 outer surface lysine residues were changed. 3D imaging indicated that all lysine residues present in iMab100 are actually located on the outer surface. 3D modelling and analysis software (InsightII) determined the spatial consequence of such replacements.

Modeller software was programmed in such a way that either cysteine bridge formation between the beta-sheets was taken into account or the cysteine bridges were neglected in analyses. All retrieved models were built with ProsaII software for more or less objective result ranking. The zp-comb parameter of ProsaII indicated the reliability of the models. Results showed that virtually all types of amino acids could replace lysine residues. However, surface exposed amino acid side chains determine the solubility of a protein. Therefore only amino acids that will solubilize the proteins were taken into account and marked with an X (see Table 14). Sequence of iMab100: underlined lysine residues were exchanged

| | |
|---|---|
| NVKLVEKGGNFVENDDDLKLTCRAEGYTIGPYCMGWFRQAPNDDSTNVATINMGGGITY | (SEQ ID NO:_) |
| YGDSVKERFDIRRDNASNTVTLSMDDLQPEDSAEYNCAGDSTIYASYYECGHGLSTGGYG | |
| YDSRGQGTDVTVSS. | |

### Example 27

Changing Amino Acids in the Exterior: Removal of Glycosylation Site.

N-glycosylation can interfere strongly with protein functions if the glycosylation site is for example present in a putative ligand-binding site. iMab100 proteins were shown to be glycosylated in Pichia pastoris cells and unable to bind to the ligand. Analysis showed that there is a putative N-glycosylation site in AR3. Inspection of the iMab100structure using template-modeling strategies with modeller software revealed that this site is potentially blocking ligand binding due to obstruction by glycosylation. This site could be removed in two different ways, by removing the residue being glycosylated or by changing the recognition motif for N-glycosylation. Here the glycosylation site itself (..RDNAS..) was removed. All residues could be used to replace the amino acid, after which ProsaII, What if and Procheck could be used to check the reliability of each individual amino acid. However, some amino acids could introduce chemical or physical properties that are unfavorable. Cysteine, for example, could make the proteins susceptible to covalent dimerization with proteins that also bear a free cysteine group. Also non-hydrophilic amino acids could disturb the folding process and were omitted. Methionine, on the other hand, is coded by ATG, which can introduce aberrant start sites in DNA sequences. The introduction of ATG sequences might result in alternative protein products due to potential alternative start sites. Methionine residues were only assessed if no other amino acids would fit. All other amino acid residues were assessed with Prosall, What if and Procheck. Replacement of N with Q was considered to be feasible and reliable.

| Protein sequence from iMab with glycosylation site: | |
|---|---|
| NVKLVEKGGNFVENDDDLKLTCRAEGYTIGPYCMGWFRQAPNDDSTNVATINMGGGITY | (SEQ ID NO:_) |
| YGDSVKERFDIRRDNASNTVTLSMDDLQPEDSAEYNCAGDSTIYASYYECGHGLSTGGYG | |
| YDSRGQGTDVTVSS. | |
| Protein sequence from iMab without glycosylation site: | |
| NVKLVEKGGNFVENDDDLKLTCRAEGYTIGPYCMGWFRQAPNDDSTNVATINMGGGITY | (SEQ ID NO:_) |
| YGDSVKERFDIRRDQASNTVTLSMDDLQPEDSAEYNCAGDSTIYASYYECGHGLSTGGYG | |
| YDSRGQGTDVTVSS. | |

Expression of iMab100in Pichia pastoris was performed by amplification of 10 ng of CM114-iMab100 DNA in a 100 microliter PCR reaction mix comprising 2 units Taq polymerase (Roche), 200 micromilor of each dNTP (Roche), buffers (Roche Taq buffer system), 2.5 micromolar of primer 107 and 108 in a Primus96 PCR machine (MWG) with the following program 25 times [94° C. 20″, 55° C. 25″, 72° C. 30″], digestion with EcoRI and NotI and ligation in EcoRI and NotI digested pPIC9 (In Vitrogen). Constructs were checked by sequencing and showed all the correct iMab100sequence. Transformation of Pichia pastoris was performed by electroporation according to the manufacturer's protocol. Growth and induction of protein expression by methanol was performed according to the manufacturer's protocol. Expression of iMab100 resulted in the production of a protein that on a SDS-PAGE showed a size of 50 kD, while expressed in E. coli the size of iMab100 is 21 kD. This difference is most likely due to glycosylation of the putative N-glycosylation site present in iMab100as described above. Therefore this glycosylation site was removed by exchange of the asparagine (N) for a glutamine (Q) in a similar way as described in example 26 except that primer 136 (Table 5) was used. This resulted in iMab115. Expression of iMab115 in E. coli resulted in the production of a 21 kD protein. ELISA experiments confirmed specificity of this iMab for lysozyme. Thus, ARs in iMab115 were positioned correctly and, more specifically, replacement of the asparagine with glutamine in AR3 did not alter AR3 properties.

### Example 28

Changing Amino Acids in the Interior of the Core: Removal of Cysteine Residues.

Obtained sequences that fold in an ig-like structure, can be used for the retrieval of similarly folded structures but aberrant amino acid sequences. Amino acids can be exchanged with other amino acids and thereby putatively changing the physical and chemical properties of the new protein if compared with the template protein. Changes on the out side of the protein structure were shown to be rather straightforward. Here, we changed amino acids that are lining up with the interior of the core. Spatial constraints of neighboring amino acid side chains and the spatial constrains of the core structure itself determine and limit the types of side chains that can be present at these locations. In addition, chemical properties of neighboring side chains can also influence the outcome of the replacements. In some replacement studies, it might be necessary to replace addition amino acids that are in close proximity of the target residues in order to obtain suitable and reliable replacements.

Here were removed the potential to form cysteine bridges in the core. The removal of only one cysteine by itself prevents the potential to form cysteine bridges in the core. However, dual replacements can also be performed in order to prevent the free cysteine to interact with other free cysteine during folding or re-folding in vivo or in vitro. First, the individual cysteine residues were replaced by any other common amino acid (19 in total). This way, 2 times 19models were retrieved. All models were assessed using ProsaII (zp-scores), What if (2^{nd} generation packing quality, backbone conformation) and Procheck (number of residues outside allowed regions). Several reliable models were obtained. Table 15 shows the zp-combined Prosa scores of the cysteine replacements at position 96. The replacement of one of the cysteines with valine was tested in vivo to validate the method. This clone was designated as iMab116 (see, Table 3) and constructed (Table 4) according to the procedure as described in Example 3. The complete iMab sequence of this clone was transferred into CM126 in the following manner. The iMab sequence, iMab116, was isolated by PCR using Cys-min iMab116 as a template together with primers pr121 and pr129 (Table 5). The resulting PCR fragment was digested with NdeI and SfiI and ligated into CM126 linearized with NdeI and SfiI. This clone, designated CM126-iMab116 was selected and used for further testing.

### Example 29

Purification of iMab116

E. coli BL21 (DE3) (Novagen) was transformed with expression vector CM126 containing an VAP insert for iMab116 containing 9 beta-strands and potentially lacking a cysteine bridge in the core (as described in Example 27). Growth and expression was similar as described in Example 5. IMab116 was purified by matrix assisted refolding similar as is described in Example 7. The purified fraction of iMab116 was analysed by SDS-PAGE as is demonstrated in FIG. 6 lane 11.

### Example 30

Specific Binding of iMab116 to Chicken Lysozyme (ELISA)

Purified iMab116(˜50 ng) was analyzed for binding to either ELK (control) and lysozyme (+ELK as a blocking agent) similar as is described in Example 8. ELISA confirmed specific binding of purified iMab116 to chicken lysozyme as is demonstrated in Table 6.

### Example 31

CD Spectra of iMab116 Proteins

IMab116 was purified as described in Example 28 and analyzed for CD spectra as described in Example 13. The spectrum of iMab116 was measured at 20° C., 95° C. and again at 20° C. to test scaffold stability and refolding characteristics. The corresponding spectra are demonstrated in FIG. 9C. The spectra measured at 20° C. were compared with the spectrum of iMab100and other 9-stranded iMab proteins at 20° C. to determine the degree of similarity of the secondary structure (see, FIG. 9J). Because the obtained spectrum is identical to the spectrum obtained from other 9 strand scaffolds, including the iMab100 spectrum, it can be concluded that the cysteine residue removal from the internal core has no effect on the structure itself.

### Example 32

Introduction of Extra Cysteine Bridge in the Core

Chemical bonding of two cysteine residues in a proteins structure (cysteine bridge) can dramatically stabilize a protein structure at temperatures below about 70 degrees Celsius. Above this temperature cysteine bridges can be broken. Some applications demand proteins that are more stable than the original protein. The spatial constrains of the core of beta strand folds as referred to in Example 1, enables cysteine bridges. This conclusion is based on the observation that in some natural occurring proteins with the referred fold a cysteine bridge is present in the center of the core (e.g., all heavy chain variable domains in antibodies). The distance between C-alpha backbone atoms of such cysteines is most often found to be between 6.3 and 7.4 angstrom.

The introduction of new cysteine residues that putatively form bridges in core motifs was analyzed by measurements. The coordinates of C-alpha atoms of a protein written in PDB files can be used to determine potential cysteine bridges. The distance between each C-alpha atom individually and all other C-alpha atoms can be calculated. The position of C-alpha atoms of the iMab100 protein obtained via comparative modeling is shown in FIG. BBB3. Insight software can be used to determine the distance between C-alpha atoms. However, standard mathematical algorithms that determine distances between two positions in space indicated by coordinates (as represented in a PDB coordinates) can also be used. Excel sheets were used to determine all possible distances. Distance values that appear to be between 6.3 and 7.4 angstrom were regarded as putative cysteine locations. Analysis indicated 33 possible cysteine bridge locations within iMab100. The cys-number indicates the position of the C-alpha atom in the structure that might be used for the insertion of a cysteine (Table 16A). However, not all positions in space are very useful; some bridges might be to close to an already available cysteine bridge, two cysteines next to each other can be problematic, two cysteine bridges between identical beta strands will not be very helpful, spatial constrains with other amino acid side chains that are located nearby. All 33models were constructed and assayed with iMab100as a template in modeller. Zp-scores of assessed models obtained with Prosall indicated that most cysteine residues are problematic. The best cysteine locations are indicated in table 16B. Two models, indicated in bold, were chosen based on the spatial position of these cysteine residues and bridges in relation to the other potential cysteine bridge. Also, some models were rejected, though the zp-scores were excellent, because of their position within the fold as reviewed with Insight (MSI).

### Example 33

Construction of an iMab100 Derivative that Contains two Extra Cysteines in the Core.

An oligonucleotide mediated site directed mutagenesis method was used to construct an iMab100 derivative, named iMab111 (Table 3), that received two extra cysteine residues. CM114-iMab100 was used as a template for the PCR reactions together with oligonucleotides pr33, pr35, pr82, pr83 (see, Table 5). In the first PCR reaction, primers pr82 and pr83 were used to generate a 401 bp fragment. In this PCR fragment a glutamine and a glycine coding residue were changed into cysteine coding sequences. This PCR fragment is used as a template in two parallel PCR reaction: In one reaction the obtained PCR fragment, CM114-iMab100 template and pr33 were used, while in the other reaction the obtained PCR fragment, CM114-iMab100 template and primers 35 were used. The former reaction gave a 584 bp product while the second one produced a 531 bp fragment. Both PCR fragments were isolated via agarose gel separation and isolation (Qiagen gel extraction kit). The products were mixed in an equimolar relation and an fragment overlap-PCR reaction with primers pr33 and pr35 resulted in a 714 bp fragment. This PCR fragment was digested with NotI and SfiI. The resulting 411 bp fragment was isolated via an agarose gel and ligated into CM114 linearized with NotI and SfiI. Sequencing analysis confirmed the product, i.e., iMab111 (Tables 4 and 3).

### Example 34

Expression of iMab111

iMab111 DNA was subcloned in CM126 as described in example 28. CM126-iMab111 transformed BL21(DE3) cells were induced with IPTG and protein was isolated as described in Example 7. Protein extracts were analyzed on 15% SDS-PAGE gels and showed a strong induction of a 21 KD protein. The expected length of iMab11 including tags is also about 21 kD indicating high production levels of this clone.

### Example 35

Purification of iMab111

E. coli BL21 (DE3) (Novagen) was transformed with expression vector CM126 containing an VAP inserts for iMab111 containing 9 beta-strands potentially containing an extra cysteine bridge (as described in Examples 32 and 33). Growth and expression was similar as described in Examples 5 and 34. iMab111 was purified by matrix assisted refolding similar as is described in Example 7. The purified fraction of iMab111 was analyzed by SDS-PAGE as is demonstrated in FIG. 6 lane 12.

### Example 36

Specific Binding of iMab111 to Chicken Lysozyme (ELISA)

Purified iMab111(˜50 ng) was analyzed for binding to either ELK (control) and lysozyme (+ELK as a blocking agent) similarly as described in Example 8. A 100-fold dilution of the protein extract in an ELISA assay resulted in a signal of approximately 20 fold higher than background signal. ELISA results confirmed specific binding of purified iMab111 to chicken lysozyme as is demonstrated in Table 6.

### Example 37

CD Spectra of iMab111 Proteins

IMab111 was purified as described in example 32 and analyzed for CD spectra as described in Example 13. The spectrum of iMab116 was measured at 20° C., 95° C. and again at 20° C. to test scaffold stability and refolding characteristics. The corresponding spectra are demonstrated in FIG. 9C. The spectra measured at 20° C. were compared with the spectrum of iMab100and other 9-stranded iMab proteins at 20° C. to determine the degree of similarity of the secondary structure (see FIG. 9J). Because the obtained spectrum is identical to the spectrum obtained from other 9 strand scaffolds, including the iMab000 spectrum, it can be concluded that the additional cysteine residue in the center of the core has no effect on the structure itself.

### Example 38

Improving Properties of Scaffolds for Specific Applications

For certain applications, the properties of a scaffold need to be optimized. For example, heat stability, acid tolerance or proteolytic stability can be advantageous or even required in certain environments in order to function well. A mutation and re-selection program can be applied to create a new scaffold with similar binding properties but with improved properties. In this example a selected binding protein is improved to resist proteolytic degradation in a proteolytic environment. New scaffolds can be tested for proteolytic resistance by a treatment with a mixture of proteases or alternatively a cascade treatment with specific protease. In addition, new scaffolds can be tested for resistance by introducing the scaffolds in the environment of the future application. In order to obtain proteolytic resistant scaffolds, the gene(s) that codes for the scaffold(s) is (are)mutated using mutagenesis methods. Next a phage display library is build from the mutated PCR products so that the new scaffolds are expressed on the outside of phages as fusion proteins with a coat protein. The phages are added to a the desired proteolytic active environment for a certain time at the desired temperature. Intact phages can be used in a standard panning procedure as described. After extensive washing bound phages are eluted, infected in E. coli cells that bear F-pili and grown overnight on a agar plate that contains appropriate antibiotics. Individual clones are re-checked for their new properties and sequenced. The process of mutation introduction and selection can be repeated several times or other selection conditions can be applied in further optimization rounds.

### Example 39

Random Mutagenesis of Scaffolds Regions

Primers annealing just 3 prime and 5 prime of the desired region (affinity regions, frameworks, loops or combinations of these) are used for amplification in the presence of dITP or dPTP as described. These mutated fragments are amplified in a second PCR reaction with primers having the identical sequence as the set of primers used in the first PCR but now containing restriction sites for recloning the fragments into the scaffold structure at the which can differ among each other in DNA sequence and thus also in protein sequence. Phage display selection procedures can be used for the retrieval of clones that have desired properties.

### Example 40

Phage Display Vector CM114-iMab100 Construction

A vector for efficient phage display (CM114-iMab100; see FIG. 4B) was constructed using part of the backbone of a pBAD (In Vitrogen). The required vector part from pBAD was amplified using primers 4 and 5 containing respectively AscI and BamHI overhanging restriction sites. In parallel a synthetic constructed fragment was made containing the sequence as described in table 4 including a new promoter, optimized g3 secretion leader, NotI site, dummy insert, SfiI site, linker, VSV-tag, trypsin specific proteolytic site, Strep-tagII and AscI site (see FIG. 4B). After combining the digested fragment and the PCR amplified pBAD vector fragment, the coding region of them 13 phage g3 core protein was amplified using AscI overhanging sites attached to primers (table 5, primer 6 and 7) and inserted after AscI digestion. Vector that contained correct sequences and correct orientations of the inserted fragments were used for further experiments.

### Example 41

Phage Display Vector CM114-iMab113 Construction

Cysteine bridges between AR4 and other affinity regions (e.g., AR1 for iMab100) can be involved in certain types of structures and stabilities that are not very likely without cysteine bridge formations. Not only can AR1 be used as an attachment for cysteines present in some affinity regions 4, but also AR2 and AR3 are obvious stabilizing sites for cysteine bridge formation. Because AR2 is an attractive alternative location for cysteine bridge formation with AR4, an expression vector is constructed which is 100% identical to CM114-iMab100 with the exception of the locations of a cysteine codon in AR2 and the lack of such in AR1. 3D-modelling analysis revealed that the best suitable location for cysteine in AR2 is at the location originally determined as a threonine (.VATIN.. into ..VACIN..). Analysis indicated that in addition to the new cysteine location (..VACIN..), the alanine residue just before the threonine residue in AR2 was replaced with a serine residue (..VSCIN..). The original cysteine in AR1 was replaced by a serine that turned out to be a suitable replacement according to 3Dmodelling analysis (Table 3).

The new determined sequence, named iMab113, (Table 4) was constructed according to the gene construction procedure as described in Example 3 and inserted in CM114 replacing iMab100.

### Example 42

Phage Display Vector CM114-iMab114 Construction

Cysteine bridges between AR4 and other regions are not always desired because intermolecular cysteine bridge formations during folding might influence the efficiency of expression and percentage of correct folded proteins. Also, in reducing environments such ARs might become less active or even inactive. Therefore, scaffolds without cysteine bridges are required.

An expression vector lacking cysteines in AR1 , 2 and 3 was constructed. This vector is 100% identical to CM114 with the exception that the cysteine in AR1 (..PYCMG..) has been changed to a serine (..PMSMG..; see Table 3). The new determined sequence, named iMab114, (table 4) was constructed according to the gene construction procedure as described above (Example 3) and inserted in CM114 replacing iMab100.

### Example 43

Amplification of Camelidae Derived CDR3 Regions

Lama pacos and Lama glama blood lymphocytes were isolated according to standard procedures as described in Spinelli et al. (Biochemistry 39 (2000) 1217-1222). RNA from these cells was isolated via Qiagen RNeasymethods according to manufacturer's protocol. cDNA was generated using muMLv or AMV (New England Biolabs) according to manufacturer's procedure. CDR3 regions from Vhh cDNA were amplified (see FIG. 10) using 1 μl cDNA reaction in 100 microliter PCR reaction mix comprising 2 units Taq polymerase (Roche), 200 μM of each dNTP (Roche), buffers (Roche Taq buffer system), 2.5 μM of forward and reverse primers in a Primus96 PCR machine (MWG) with the following program 35 times [94° C. 20 ″, 50° C. 25 ″, 72° C. 30″]. In order to select for CDR3 regions containing at least one cysteine primer 56 (table 5) was used as a forward primer and in case to select for CDR regions that do not contain cysteines primer 76 (table 5) was used in the first PCR round. In both cases primer 16 (Table 5) was used as reverse primer. Products were separated on a 1% Agarose gel and products of the correct length (˜250 bp) were isolated and purified using Qiagen gel extraction kit. 5μl of these products were used in a next round of PCR similar as described above in which primer 8 (Table 5) and primer 9 (Table 5) were used to amplify CDR3 regions. Products were separated on a 2% Agarose gel and products of the correct length (˜80-150 bp) were isolated and purified using Qiagen gel extraction kit. In order to adapt the environment of the camelidae CDR3 regions to scaffold iMab100 two extra rounds of PCR similar to the first PCR method was performed on 5 μl of the products with the exception that the cycle number was decreased to 15 cycles and in which primer 73 (Table 5) and 75 (Table 5) were subsequently used as forward primer and primer 49 (Table 5) was used as reverse primer.

### Example 44

Amplification of Cow Derived CDR3 Regions

Cow (Bos taurus) blood lymphocytes were isolated according to standard procedures as described in Spinelli et al. (Biochemistry 39 (2000) 1217-1222). RNA from these cells was isolated via Qiagen RNeasymethods according to manufacturer's protocol. cDNA was generated usingmuMLv or AMV (New England Biolabs) according to manufacturer's procedure. CDR3 regions from Vh cDNA was amplified using 1 μl cDNA reaction in 100 microliter PCR reactionmix comprising 2 units Taq polymerase (Roche), 200 μM of each dNTP (Roche), buffers (Roche Taq buffer system), 2.5 μM of primer 299 (table 5) and 300 (table 5) in a Primus96 PCRmachine (MWG) with the following program 35 times [94° C. 20″, 50° C. 25″, 72° C. 30″]. Products were separated on a 2% Agarose gel and products of the correct length were isolated and purified using Qiagen gel extraction kit. The length distribution of the PCR products observed (see FIG. 11) represents the average length of cow CDR3 regions. Correcting for framework sequences that are present in primer 299 (21 amino acids; table 5) and 300 (27 amino acids; Table 5) it can be concluded that the average length of cow CDR3s is: 120 base average PCR product lengthminus 48 base frameworks determines 72 bases and thus 24 amino acids. This result corresponds very well with the results observed by Spinelli et al. (Biochemistry 39 (2000) 1217-1222). These CDR regions are therefore extremely useful for naive library constructions.

Isolated and purified products can be used to adapt the sequences around the actual CDR3/AR4 location in a way that the coding regions of the frameworks are gradually adapted via several PCR modifications rounds similarly as described for lama derived ARs (see, Example 43).

### Example 45

Libraries Containing Loop Variegations in AR4 by Insertion of Amplified CDR3 Regions

A nucleic acid phage display library having variegations in AR4 was prepared by the following method. Amplified CDR3 regions from lama's immunized with lactoperoxidase and lactoferrin was obtained as described in example 43 and were digested with PstI and KpnI and ligated with T4 DNA ligase into the PstI and KpnI digested and alkaline phosphatase treated vector CM114-iMab113 or CM114-iMab114. Cysteine containing CDR3s were cloned into CM114-iMab114 while CDR3s without cysteines were cloned into vector CM114-iMab113. The libraries were constructed by electroporation into E. coli TG1 electrocompetent cells by using a BTX electrocell manipulator ECM630. Cells were recovered in SOB and grown on plates that contained 4% glucose, 100 micrograms ampicillin per milliliter in 2*TY-agar. After overnight culture at 37° C., cells were harvested in 2*TYmedium and stored in 50% glycerol as concentrated dispersions at -80° C. Typically, 5×10⁸ transformants were obtained with 1 μg DNA and a library contained about 10⁹ independent clones.

### Example 46

Libraries Containing Loop Variegations in AR4 by Insertion of Randomized CDR3 Regions

A nucleic acid phage display library having variegations in AR4 by insertion of randomized CDR3 regions was prepared by the following method. CDR3 regions from non-immunized and immunized lama's were amplified as described in example 28 except that in the second PCR round dITP according to Spee et al. (1993) or dPTP according to Zaccolo et al. (1996) were included as described in example 35. Preparation of the library was performed as described in example 28. With dITP amutation rate of 2% was achieved while with dPTP included in the PCR a mutation rate of over 20% was obtained.

### Example 47

Enrichment of VAPs that Bind to Target Molecules

About 50 microliter of the library stocks was inoculated in 50 ml 2*TY/100 microgram ampicillin/4% glucose and grown until an OD600 of 0.5 was reached. Next 10¹¹ VCSM13 (Stratagene) helper phages were added. The culture was left at 37° C. without shaking for 45 minutes to enable infection. Cells were pellet by centrifugation and the supernatant was discarded. Pellets were resuspended in 400 ml 2*TY/100 microgram ampicillin and cultured for 1 hour at 37° C. after which 50 μg/ml kanamycin was added. Infected cultures were grown at 30° C. for 8 hours on a 200 rpm shaking platform. Next, bacteria were removed by pelleting at 5000 g at 4° C. for 30 minutes. The supernatant was filtered through a 0.45 micrometer PVDF filtermembrane. Poly-ethylene-glycol and NaCl were added to the flow through with final concentrations of respectively 4% and 0.5 M. In this way phages precipitated on ice and were pelleted by centrifugation at 6000 g. The phage pellet was solved in 50% glycerol/50% PBS and stored at −20° C.

The selection of phage-displayed VAPs was performed as follows. Approximately 1 μg of a target molecule (antigen) was immobilized in an immunotube (Nunc) ormicrotiter plate (Nunc) in 0.1 m sodium carbonate buffer (pH 9.4) at 4° C. o/n. After the removal of this solution, the tubes were blocked with a 3% skimmilk powder solution (ELK) in PBS or a similar blocking agent for at least 2 hrs either at room temperature or at 4° C. o/n. After removal of the blocking agent a phagemid library solution containing approximately 10¹²-10¹³ colony forming units (cfu), which was pre-blocked with blocking buffer for 1 hour at room temperature, was added in blocking buffer. Incubation was performed on a slow rotating platform for 1 hour at room temperature. The tubes were then washed three times with PBS, two times with PBS with 0.1% Tween and again four times with PBS. Bound phages were eluted with an appropriate elution buffer, either 300 μl 0.1 m glycine pH 2.2 or 500 μl 0.1% trypsin in PBS. Recovered phages were immediately neutralized with 700 μl 1 m Tris-HCl pH 8.5 if eluted with glycine. Alternatively the bound phages were eluted by incubation with PBS containing the antigen (1-10 μM). Recovered phages were amplified as described above employing E.coli XLI-Blue (Stratagene) or Top10F′ (In Vitrogen) cells as the host. The selection process was repeated several times to concentrate positive clones. After the final round, individual clones were picked and their binding affinities and DNA sequences were determined.

The binding affinities of VAPs were determined by ELISA as described in Example 6, either as gIII-fusion protein on the phage particles or after subcloning as a NdeI-SfiI into the expression vector CM126 as described in Example 4. E. coli BL21(DE3) or Origami(DE3) (Novagen) were transformed by electroporation as described in Example 5 and transformants were grown in 2×TY medium supplemented with ampicillin (100 μg/ml). When the cell cultures reached an OD600 ˜1 protein expression was induced by adding IPTG (0.2 mM). After 4 hours at 37° C., cells were harvested by centrifugation. Proteins were isolated as described in Example 5.

### Example 48

Enrichment for Lactoferrin Binding VAPs

Purified Lactoferrin (LF) was Supplied by DMV-Campina.

A phage display library with variegations in AR4 as described in example 45 was used to select LF binding VAPs. LF (10 microgram in 1 ml sodium bicarbonate buffer (0.1 m, pH 9.4)) was immobilized in an immunotube (Nunc) followed by blocking with 3% chicken serum in PBS. Panning was performed as described in Example 32. 10¹³ phages were used as input. After the 1^{st} round of panning about 10000 colonies were formed. After the 2^{nd} panning round 500 to 1000 colonies were formed. Individual clones were grown and VAPs were produced and checked by ELISA as described in Example 6. Enrichment was found for clones with the following AR4: CAAQTGGPPAPYYCTEYGSPDSW (SEQ ID NO:_).

### Example 49

Enrichment for Lactoperoxidase Binding VAPs

Purified Lactoperoxidase (LP) was Supplied by DMV-Campina.

A phage display library with variegations in AR4 as described in example 45 was used to select LP binding VAPs. LP (10 microgram in 1 ml sodium bicarbonate buffer (0.1 m, pH 9.4)) was immobilized in an immunotube (Nunc) followed by blocking with 3% chicken serum in PBS. Panning was performed as described in example 32 10¹³ phages were used as input. After the 1^{St} round of panning about 5000 colonies were formed. After the ₂nd panning round 500 to 1000 colonies were formed. Individual clones were grown and VAPs were produced and checked by ELISA as described in Example 6. Positive clones were sequenced. Enrichment was found for clones with the following AR4:

| | |
|---|---|
| CAAVLGCGYCDYDDGDVGSW | (SEQ ID NO:_) |
| CAATENFRIAREGYEYDYW | (SEQ ID NO:_) |
| CAATSDFRIAREDYEYDYW | (SEQ ID NO:_) |

### Example 50

RNase A Binder, Construction, Maturation and Panning.

A synthetic RNase A binding iMab, iMab130, was synthesized as described in Example 3 (Table 4, Table 3) and subsequently cloned into CM114 forming CM114-iMab130. Chimeric phages with iMab130 as a fusion protein with the g3 coat protein were produced under conditions as described for library amplification procedure in Example 32 Panning with these chirmeric phages against RNase A coated immunotubes (see, Example 32 for panning procedure) failed to show RNase A specific binding of iMab130. Functional positioning of the RNase A binding regions had clearly failed, probably due to minor distortions of surrounding amino acid side chains. Small modifications of the scaffold might help to displace ARs into correct positions. In order to achieve this, the iMab130 coding region was mutated using the following method: iMab130 present in vector CM114 was mutagenised using either dITP or dPTP during amplification of the scaffold with primers 120 and 121 (table . . . ).mutagenizing concentrations of 1.7 mM dITP or 300 μM, 75 μM or 10 μM dPTP were used. Resulting PCR products were isolated from an agarose gel via Qiagen's gel elution system according to manufacturer's procedures. Isolated products were amplified in the presence of 100 μM of dNTPs (Roche) in order to generate dITP and dPTP free products. After purification via Qiagen's PCR clean up kit, these PCR fragments were digested with NotI and SfiI (NEB) and ligated into NotI and SfiI linearized CM114. Precipitated and 70% ethanol washed ligation products were transformed into TG1 bymeans of electroporation and grown in 2×TYmedium containing 100 μg/ml ampicillin and 2% glucose and subsequently infected with VCSM13 helper phage (Stratagene) for chimeric phage production as described in example 32. Part of the transformation was plated on 2×TY plates containing 2% glucose and 100 microgram/ml ampicillin to determine transformation frequency:

These phage libraries were used in RNase A panning experiments as described in example 32 RNase A was immobilized in immunotubes and panning was performed. After panning, phages were eluted and used for infection of TOP10 F′ (In Vitrogen), and grown overnight at 37° C. on 2×TY plates containing 2% glucose and 100 μg/ml ampicillin and 25 microgram/ml tetracycline. The number of retrieved colonies is indicated in Table 17. As can be concluded from the number of colonies obtained after panning with phage libraries derived from different mutagenesis levels of iMab130, a significant increase of binders can be observed from the library with a mild mutagenesis level, being dITP (Table 17)

### Example 51

Immobilisation Procedure

1 g of epoxy activated Sepharose 6B (manufacturer Amersham Biosciences) was packed in a column and washed with 10 bed volumes coupling buffer (200 mM potassium phosphate, pH 7). The protein to be coupled was dissolved in coupling buffer at a concentration of 1 mg/ml and passed over the column at a flow rate of 0.1 ml/min. After passing 20 bed volumes of protein solution, the column was washed with coupling buffer. Passing 10 bed volumes of 0.2 M ethanolamine/ 200 mM potassium phosphate pH 7 blocked the unreacted epoxy groups. The resin was then washed with 20 bed volumes of 50 mM potassium phosphate pH 7 after which it was ready for use.

### Example 52

iMab100 Purification via Lysozyme Immobilized Beads

Lysozyme was immobilized on Eupergit, an activated epoxy-resin from Rohm and used in a column. A solution containing iMab100was passed on the column and the concentration was measured in a direct bypass and the flow through from the column (A280 nm). The difference indicated the amount of iMab100that was bound to the column. The bound iMab100could be released with a CAPS buffer pH11. Control experiments with BSA indicated that the binding of iMab100 to immobilized lysozyme was specific.

### Example 53

Lysozyme Purification via iMab100 Immobilized Beads

iMab100 was immobilized on Eupergit and used in a column. A solution containing Lysozyme was passed on the column and the concentration was measured and in a direct bypass and the flow through from the column (A280 nm). The difference indicated the amount of Lysozyme that was bound to the column. The bound Lysozyme could be released with a CAPS buffer pH11. Control experiments with BSA indicated that the binding of Lysozyme to immobilized iMab100 was specific.

### Example 54

Stability of iMab100 in Whey Fractions

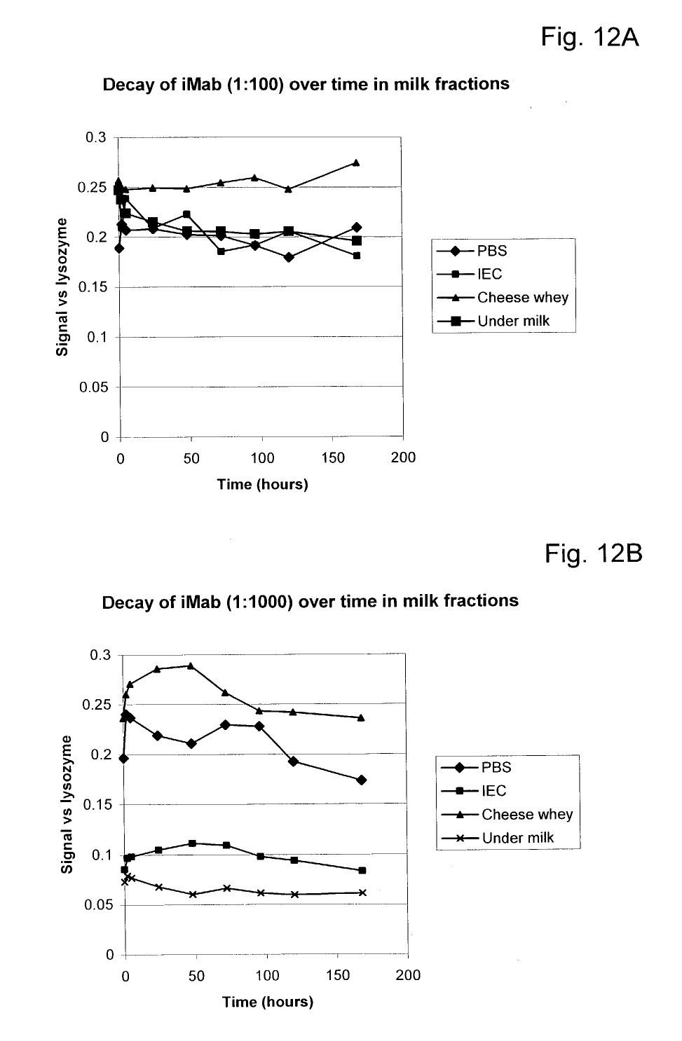

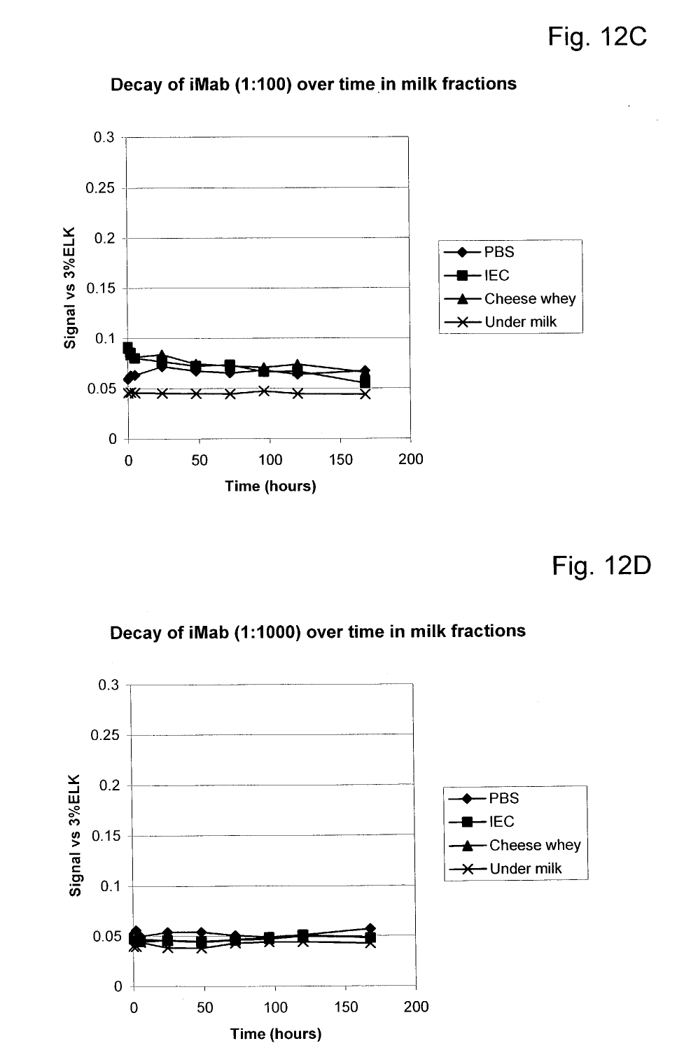

The stability of iMab100 in several milk fractions was measured by lysozyme coated plates via ELISA methods (Example 8). If the tags, scaffold regions or affinity regions were proteolytically degraded, a decreased anti-lysozyme activity would be observed. iMab100was diluted in several different solution: 1×PBS as a control, ion-exchange fraction from cheese-whey, gouda-cheese-whey and low Pasteurized undermilk, 1.4 μm filtered to a final concentration of 40 μg/ml. All fractions were stored at 8° C., samples were taken after:0,2 and 5 hours and after 1, 2, 3, 4, 5 and 7 days. Samples were placed at −20° C. to prevent further degradation. ELISA detection was performed as described in Example 8 and shown in FIG. 12. The activity pattern of iMab100 remained similar throughout the experiment. Therefore, it can be concluded that iMab100, including the tags, were stable in assayed milk fractions.

TABLE 1
Examples of nine stranded (strands-only) of in PDB format

| | | | | | | | | | | |
|---|---|---|---|---|---|---|---|---|---|---|
| 1Neu | | | | | | | | | | |
| ATOM | 1 | CA | GLY | 2 | −9.450 | −13.069 | 10.671 | 1.00 | 25.06 | C |
| ATOM | 2 | CA | GLY | 3 | −9.868 | −10.322 | 8.019 | 1.00 | 20.77 | C |
| ATOM | 3 | CA | GLY | 4 | −6.884 | −9.280 | 5.813 | 1.00 | 19.01 | C |
| ATOM | 4 | CA | GLY | 5 | −6.047 | −5.991 | 4.016 | 1.00 | 19.75 | C |
| ATOM | 5 | CA | GLY | 6 | −2.638 | −4.349 | 3.125 | 1.00 | 22.33 | C |
| ATOM | 6 | CA | GLY | 7 | −1.382 | −1.720 | 5.647 | 1.00 | 24.80 | C |
| ATOM | 7 | CA | GLY | 8 | −0.685 | 1.080 | 3.150 | 1.00 | 28.23 | C |
| ATOM | 8 | CA | GLY | 9 | −0.917 | 1.393 | −0.623 | 1.00 | 26.37 | C |
| ATOM | 9 | CA | GLY | 10 | 0.737 | 3.923 | −2.887 | 1.00 | 29.08 | C |
| ATOM | 10 | CA | GLY | 11 | −0.741 | 5.082 | −6.156 | 1.00 | 26.48 | C |
| ATOM | 11 | CA | GLY | 12 | 0.157 | 7.450 | −8.989 | 1.00 | 27.26 | C |
| ATOM | 12 | CA | GLY | 13 | −2.216 | 10.173 | −10.146 | 1.00 | 26.73 | C |
| ATOM | 13 | CA | GLY | 15 | −3.567 | 5.434 | −12.371 | 1.00 | 23.37 | C |
| ATOM | 14 | CA | GLY | 16 | −5.492 | 2.682 | −10.482 | 1.00 | 22.86 | C |
| ATOM | 15 | CA | GLY | 17 | −4.920 | 0.709 | −7.288 | 1.00 | 20.21 | C |
| ATOM | 16 | CA | GLY | 18 | −6.462 | −2.512 | −5.933 | 1.00 | 19.23 | C |
| ATOM | 17 | CA | GLY | 19 | −7.735 | −2.659 | −2.366 | 1.00 | 17.13 | C |
| ATOM | 18 | CA | GLY | 20 | −7.524 | −6.278 | −1.141 | 1.00 | 19.06 | C |
| ATOM | 19 | CA | GLY | 21 | −9.914 | −7.812 | 1.355 | 1.00 | 15.28 | C |
| ATOM | 20 | CA | GLY | 22 | −10.325 | −11.479 | −2.238 | 1.00 | 15.88 | C |
| ATOM | 21 | CA | GLY | 23 | −11.233 | −13.572 | 5.249 | 1.00 | 18.12 | C |
| ATOM | 22 | CA | GLY | 24 | −10.228 | −16.988 | 6.550 | 1.00 | 18.67 | C |
| ATOM | 23 | CA | GLY | 25 | −11.569 | −19.457 | 9.107 | 1.00 | 20.29 | C |
| ATOM | 24 | CA | GLY | 33 | −21.431 | −13.432 | 3.640 | 1.00 | 24.64 | C |
| ATOM | 25 | CA | GLY | 34 | −21.423 | −9.665 | 3.090 | 1.00 | 21.24 | C |
| ATOM | 26 | CA | GLY | 35 | −18.613 | −7.157 | 2.347 | 1.00 | 17.90 | C |
| ATOM | 27 | CA | GLY | 36 | −18.908 | −3.427 | 3.018 | 1.00 | 16.78 | C |
| ATOM | 28 | CA | GLY | 37 | −16.219 | −0.829 | 2.103 | 1.00 | 15.77 | C |
| ATOM | 29 | CA | GLY | 38 | −16.123 | 2.573 | 3.946 | 1.00 | 16.15 | C |
| ATOM | 30 | CA | GLY | 39 | −13.870 | 5.619 | 3.251 | 1.00 | 17.11 | C |
| ATOM | 31 | CA | GLY | 40 | −12.494 | 8.314 | 5.642 | 1.00 | 19.95 | C |
| ATOM | 32 | CA | GLY | 46 | −16.461 | 10.313 | 9.058 | 1.00 | 25.44 | C |
| ATOM | 33 | CA | GLY | 47 | −16.556 | 6.820 | 7.445 | 1.00 | 21.65 | C |
| ATOM | 34 | CA | GLY | 48 | −18.735 | 6.834 | 4.274 | 1.00 | 18.17 | C |
| ATOM | 35 | CA | GLY | 49 | −19.877 | 3.539 | 2.632 | 1.00 | 16.87 | C |
| ATOM | 36 | CA | GLY | 50 | −18.781 | 3.271 | −1.024 | 1.00 | 16.20 | C |
| ATOM | 37 | CA | GLY | 51 | −19.542 | −0.410 | −1.819 | 1.00 | 18.17 | C |
| ATOM | 38 | CA | GLY | 58 | −23.016 | −6.057 | −5.656 | 1.00 | 25.27 | C |
| ATOM | 39 | CA | GLY | 59 | −24.037 | −2.432 | −4.897 | 1.00 | 25.25 | C |
| ATOM | 40 | CA | GLY | 60 | −21.801 | 0.483 | −5.960 | 1.00 | 27.11 | C |
| ATOM | 41 | CA | GLY | 61 | −22.391 | 4.033 | −4.746 | 1.00 | 33.08 | C |
| ATOM | 42 | CA | GLY | 69 | −14.428 | 4.962 | −10.168 | 1.00 | 19.37 | C |
| ATOM | 43 | CA | GLY | 70 | −15.191 | 1.646 | −8.389 | 1.00 | 17.40 | C |
| ATOM | 44 | CA | GLY | 71 | −14.920 | −1.883 | −9.862 | 1.00 | 17.48 | C |
| ATOM | 45 | CA | GLY | 72 | −15.954 | −5.092 | −8.038 | 1.00 | 17.29 | C |
| ATOM | 46 | CA | GLY | 73 | −13.296 | −7.784 | −8.446 | 1.00 | 17.82 | C |
| ATOM | 47 | CA | GLY | 74 | −13.990 | −10.198 | −5.611 | 1.00 | 20.26 | C |
| ATOM | 48 | CA | GLY | 81 | −14.142 | −8.971 | −1.381 | 1.00 | 15.95 | C |
| ATOM | 49 | CA | GLY | 82 | −11.604 | −6.836 | −3.256 | 1.00 | 14.51 | C |
| ATOM | 50 | CA | GLY | 83 | −12.322 | −3.672 | −5.337 | 1.00 | 14.59 | C |
| ATOM | 51 | CA | GLY | 84 | −10.287 | −1.441 | −7.646 | 1.00 | 15.51 | C |
| ATOM | 52 | CA | GLY | 85 | −10.204 | 2.403 | −7.291 | 1.00 | 16.32 | C |
| ATOM | 53 | CA | GLY | 86 | −9.791 | 3.931 | −10.768 | 1.00 | 17.40 | C |
| ATOM | 54 | CA | GLY | 87 | −8.338 | 7.203 | −12.126 | 1.00 | 20.53 | C |
| ATOM | 55 | CA | GLY | 89 | −6.478 | 11.899 | −7.844 | 1.00 | 33.26 | C |
| ATOM | 56 | CA | GLY | 90 | −4.328 | 13.752 | −5.293 | 1.00 | 33.41 | C |
| ATOM | 57 | CA | GLY | 91 | −7.275 | 14.318 | −3.027 | 1.00 | 28.24 | C |
| ATOM | 58 | CA | GLY | 92 | −8.033 | 10.627 | −2.715 | 1.00 | 23.61 | C |
| ATOM | 59 | CA | GLY | 93 | −5.654 | 10.161 | 0.314 | 1.00 | 22.12 | C |
| ATOM | 60 | CA | GLY | 94 | −7.413 | 8.486 | 3.205 | 1.00 | 18.21 | C |
| ATOM | 61 | CA | GLY | 95 | −8.183 | 5.294 | 5.047 | 1.00 | 18.57 | C |
| ATOM | 62 | CA | GLY | 96 | −10.462 | 2.502 | 3.647 | 1.00 | 17.72 | C |
| ATOM | 63 | CA | GLY | 97 | −12.048 | −0.109 | 5.899 | 1.00 | 18.72 | C |
| ATOM | 64 | CA | GLY | 98 | −13.364 | −3.549 | 4.893 | 1.00 | 18.53 | C |
| ATOM | 65 | CA | GLY | 99 | −16.169 | −4.934 | 7.135 | 1.00 | 18.45 | C |
| ATOM | 66 | CA | GLY | 100 | −17.005 | −8.651 | 6.806 | 1.00 | 17.71 | C |
| ATOM | 67 | CA | GLY | 108 | −18.629 | −7.767 | 11.674 | 1.00 | 32.51 | C |
| ATOM | 68 | CA | GLY | 109 | −14.846 | −7.953 | 11.718 | 1.00 | 28.62 | C |
| ATOM | 69 | CA | GLY | 110 | −12.921 | −5.079 | 10.098 | 1.00 | 23.31 | C |
| ATOM | 70 | CA | GLY | 111 | −9.483 | −4.260 | 8.692 | 1.00 | 19.82 | C |
| ATOM | 71 | CA | GLY | 112 | −8.175 | −1.005 | 7.171 | 1.00 | 19.75 | C |
| ATOM | 72 | CA | GLY | 113 | −5.635 | 0.154 | 4.554 | 1.00 | 18.42 | C |
| ATOM | 73 | CA | GLY | 114 | −4.325 | 3.731 | 4.046 | 1.00 | 18.70 | C |
| ATOM | 74 | CA | GLY | 115 | −3.915 | 5.265 | 0.576 | 1.00 | 19.95 | C |
| ATOM | 75 | CA | GLY | 116 | −1.274 | 7.843 | −0.510 | 1.00 | 25.78 | C |
| ATOM | 76 | CA | GLY | 117 | −1.158 | 9.173 | −4.076 | 1.00 | 31.06 | C |
| ATOM | 77 | CA | GLY | 118 | 1.962 | 10.836 | −5.500 | 1.00 | 38.60 | C |
| ATOM | 78 | CA | GLY | 119 | 3.251 | 11.884 | −8.972 | 1.00 | 42.50 | C |
| TER | | | | | | | | | | |
| 1MEL | | | | | | | | | | |
| ATOM | 79 | CA | GLY A | 3 | −9.610 | −12.241 | 11.306 | 1.00 | 36.96 | C |
| ATOM | 80 | CA | GLY A | 4 | −9.672 | −9.746 | 8.420 | 1.00 | 28.80 | C |
| ATOM | 81 | CA | GLY A | 5 | −6.352 | −8.965 | 6.761 | 1.00 | 31.64 | C |
| ATOM | 82 | CA | GLY A | 6 | −6.225 | −6.212 | 4.154 | 1.00 | 24.68 | C |
| ATOM | 83 | CA | GLY A | 7 | −3.391 | −5.808 | 1.630 | 1.00 | 21.60 | C |
| ATOM | 84 | CA | GLY A | 8 | −2.679 | −3.267 | −1.055 | 1.00 | 17.27 | C |
| ATOM | 85 | CA | GLY A | 9 | −2.837 | 0.450 | −0.715 | 1.00 | 18.58 | C |
| ATOM | 86 | CA | GLY A | 10 | 0.112 | 2.848 | −0.895 | 1.00 | 16.78 | C |
| ATOM | 87 | CA | GLY A | 11 | 0.663 | 5.945 | −3.023 | 1.00 | 11.36 | C |
| ATOM | 88 | CA | GLY A | 12 | −0.001 | 6.528 | −6.640 | 1.00 | 8.84 | C |
| ATOM | 89 | CA | GLY A | 13 | −0.394 | 9.450 | −8.944 | 1.00 | 11.16 | C |
| ATOM | 90 | CA | GLY A | 14 | −3.805 | 10.880 | −9.667 | 1.00 | 10.62 | C |
| ATOM | 91 | CA | GLY A | 16 | −3.498 | 5.586 | −11.682 | 1.00 | 7.90 | C |
| ATOM | 92 | CA | GLY A | 17 | −5.029 | 2.507 | −10.132 | 1.00 | 7.54 | C |
| ATOM | 93 | CA | GLY A | 18 | −4.536 | 0.406 | −6.998 | 1.00 | 6.47 | C |
| ATOM | 94 | CA | GLY A | 19 | −5.823 | −2.962 | −5.868 | 1.00 | 5.43 | C |
| ATOM | 95 | CA | GLY A | 20 | −6.773 | −3.739 | −2.263 | 1.00 | 8.09 | C |
| ATOM | 96 | CA | GLY A | 21 | −7.534 | −7.231 | −0.907 | 1.00 | 8.76 | C |
| ATOM | 97 | CA | GLY A | 22 | −9.056 | −8.745 | 2.173 | 1.00 | 8.33 | C |
| ATOM | 98 | CA | GLY A | 23 | −9.100 | −12.281 | 3.393 | 1.00 | 13.77 | C |
| ATOM | 99 | CA | GLY A | 24 | −11.485 | −13.177 | 6.222 | 1.00 | 17.53 | C |
| ATOM | 100 | CA | GLY A | 25 | −9.970 | −15.910 | 8.360 | 1.00 | 23.23 | C |
| ATOM | 101 | CA | GLY A | 26 | −11.324 | −17.985 | 11.176 | 1.00 | 25.25 | C |
| ATOM | 102 | CA | GLY A | 32 | −22.537 | −12.961 | 4.478 | 1.00 | 5.00 | C |
| ATOM | 103 | CA | GLY A | 33 | −21.061 | −9.574 | 3.450 | 1.00 | 4.64 | C |
| ATOM | 104 | CA | GLY A | 34 | −17.557 | −8.097 | 2.923 | 1.00 | 2.48 | C |
| ATOM | 105 | CA | GLY A | 35 | −17.173 | −4.447 | 1.958 | 1.00 | 2.00 | C |
| ATOM | 106 | CA | GLY A | 36 | −14.942 | −1.420 | 2.147 | 1.00 | 3.40 | C |
| ATOM | 107 | CA | GLY A | 37 | −15.248 | 1.775 | 4.153 | 1.00 | 6.64 | C |
| ATOM | 108 | CA | GLY A | 38 | −12.980 | 4.768 | 4.009 | 1.00 | 8.74 | C |
| ATOM | 109 | CA | GLY A | 39 | −12.174 | 7.634 | 6.381 | 1.00 | 19.24 | C |
| ATOM | 110 | CA | GLY A | 44 | −17.378 | 11.364 | 7.293 | 1.00 | 26.93 | C |
| ATOM | 111 | CA | GLY A | 45 | −16.728 | 7.653 | 7.090 | 1.00 | 16.63 | C |
| ATOM | 112 | CA | GLY A | 46 | −17.836 | 6.562 | 3.651 | 1.00 | 13.26 | C |
| ATOM | 113 | CA | GLY A | 47 | −19.278 | 3.220 | 2.664 | 1.00 | 8.30 | C |
| ATOM | 114 | CA | GLY A | 48 | −17.484 | 2.453 | −0.627 | 1.00 | 6.32 | C |
| ATOM | 115 | CA | GLY A | 49 | −18.387 | −0.943 | −1.936 | 1.00 | 3.71 | C |
| ATOM | 116 | CA | GLY A | 57 | −24.217 | −9.042 | −5.792 | 1.00 | 11.04 | C |
| ATOM | 117 | CA | GLY A | 58 | −22.300 | −5.759 | −5.582 | 1.00 | 7.10 | C |
| ATOM | 118 | CA | GLY A | 59 | −23.147 | −2.237 | −4.440 | 1.00 | 9.00 | C |
| ATOM | 119 | CA | GLY A | 60 | −21.067 | 0.930 | −4.751 | 1.00 | 8.36 | C |
| ATOM | 120 | CA | GLY A | 61 | −21.147 | 4.392 | −3.266 | 1.00 | 15.85 | C |
| ATOM | 121 | CA | GLY A | 67 | −14.348 | 3.577 | −11.091 | 1.00 | 12.68 | C |
| ATOM | 122 | CA | GLY A | 68 | −14.176 | 0.900 | −8.416 | 1.00 | 8.15 | C |
| ATOM | 123 | CA | GLY A | 69 | −15.003 | −2.767 | −8.799 | 1.00 | 8.39 | C |
| ATOM | 124 | CA | GLY A | 70 | −15.301 | −5.266 | −5.949 | 1.00 | 5.47 | C |
| ATOM | 125 | CA | GLY A | 71 | −15.018 | −8.998 | −6.510 | 1.00 | 8.48 | C |
| ATOM | 126 | CA | GLY A | 72 | −14.299 | −12.215 | −4.617 | 1.00 | 18.00 | C |
| ATOM | 127 | CA | GLY A | 79 | −12.288 | −10.021 | −1.938 | 1.00 | 9.55 | C |
| ATOM | 128 | CA | GLY A | 80 | −10.619 | −7.230 | −3.968 | 1.00 | 4.94 | C |
| ATOM | 129 | CA | GLY A | 81 | −11.319 | −3.691 | −4.814 | 1.00 | 4.92 | C |
| ATOM | 130 | CA | GLY A | 82 | −9.808 | −2.556 | −8.096 | 1.00 | 7.44 | C |
| ATOM | 131 | CA | GLY A | 83 | −9.608 | 1.233 | −8.010 | 1.00 | 6.55 | C |
| ATOM | 132 | CA | GLY A | 84 | −9.109 | 2.986 | −11.359 | 1.00 | 9.49 | C |
| ATOM | 133 | CA | GLY A | 85 | −9.157 | 6.689 | −12.211 | 1.00 | 12.54 | C |
| ATOM | 134 | CA | GLY A | 87 | −8.265 | 11.163 | −7.900 | 1.00 | 15.46 | C |
| ATOM | 135 | CA | GLY A | 88 | −6.724 | 12.875 | −4.855 | 1.00 | 13.94 | C |
| ATOM | 136 | CA | GLY A | 89 | −10.223 | 13.046 | −3.286 | 1.00 | 18.68 | C |
| ATOM | 137 | CA | GLY A | 90 | −9.896 | 9.253 | −2.924 | 1.00 | 9.55 | C |
| ATOM | 138 | CA | GLY A | 91 | −7.043 | 9.782 | −0.407 | 1.00 | 6.54 | C |
| ATOM | 139 | CA | GLY A | 92 | −8.201 | 8.111 | 2.809 | 1.00 | 6.38 | C |
| ATOM | 140 | CA | GLY A | 93 | −7.841 | 5.198 | 5.205 | 1.00 | 6.33 | C |
| ATOM | 141 | CA | GLY A | 94 | −9.509 | 2.130 | 3.746 | 1.00 | 6.08 | C |
| ATOM | 142 | CA | GLY A | 95 | −11.083 | −0.367 | 6.028 | 1.00 | 10.12 | C |
| ATOM | 143 | CA | GLY A | 96 | −12.264 | −3.845 | 5.241 | 1.00 | 10.76 | C |
| ATOM | 144 | CA | GLY A | 97 | −15.411 | −5.059 | 6.995 | 1.00 | 9.08 | C |
| ATOM | 145 | CA | GLY A | 98 | −17.534 | −8.225 | 7.154 | 1.00 | 8.28 | C |
| ATOM | 146 | CA | GLY A | 122 | −15.862 | −7.486 | 11.967 | 1.00 | 15.49 | C |
| ATOM | 147 | CA | GLY A | 123 | −13.351 | −4.917 | 11.000 | 1.00 | 12.37 | C |
| ATOM | 148 | CA | GLY A | 124 | −9.811 | −4.988 | 9.801 | 1.00 | 14.34 | C |
| ATOM | 149 | CA | GLY A | 125 | −6.730 | −2.842 | 10.015 | 1.00 | 22.49 | C |
| ATOM | 150 | CA | GLY A | 126 | −6.910 | 0.334 | 7.957 | 1.00 | 17.72 | C |
| ATOM | 151 | CA | GLY A | 127 | −4.732 | 0.802 | 4.921 | 1.00 | 16.51 | C |
| ATOM | 152 | CA | GLY A | 128 | −3.822 | 4.319 | 3.804 | 1.00 | 16.84 | C |
| ATOM | 153 | CA | GLY A | 129 | −4.119 | 5.119 | 0.160 | 1.00 | 11.90 | C |
| ATOM | 154 | CA | GLY A | 130 | −2.710 | 8.445 | −0.901 | 1.00 | 8.75 | C |
| ATOM | 155 | CA | GLY A | 131 | −3.277 | 9.842 | −4.344 | 1.00 | 14.37 | C |
| ATOM | 156 | CA | GLY A | 132 | −0.480 | 12.243 | −5.478 | 1.00 | 23.32 | C |
| ATOM | 157 | CA | GLY A | 133 | −0.447 | 15.425 | −7.580 | 1.00 | 36.14 | C |
| TER | | | | | | | | | | |
| 1F97 | | | | | | | | | | |
| ATOM | 158 | CA | GLY A | 29 | −9.830 | −13.499 | 10.551 | 1.00 | 41.25 | C |
| ATOM | 159 | CA | GLY A | 30 | −9.746 | −10.552 | 8.150 | 1.00 | 22.43 | C |
| ATOM | 160 | CA | GLY A | 31 | −6.475 | −9.224 | 6.722 | 1.00 | 24.73 | C |
| ATOM | 161 | CA | GLY A | 32 | −4.787 | −7.203 | 3.981 | 1.00 | 20.95 | C |
| ATOM | 162 | CA | GLY A | 33 | −1.574 | −7.581 | 1.983 | 1.00 | 28.77 | C |
| ATOM | 163 | CA | GLY A | 34 | −0.760 | −3.875 | 2.262 | 1.00 | 33.48 | C |
| ATOM | 164 | CA | GLY A | 35 | −2.198 | −1.487 | 4.855 | 1.00 | 27.47 | C |
| ATOM | 165 | CA | GLY A | 36 | −0.223 | 1.510 | 3.544 | 1.00 | 29.20 | C |
| ATOM | 166 | CA | GLY A | 37 | −0.984 | 1.885 | −0.160 | 1.00 | 23.99 | C |
| ATOM | 167 | CA | GLY A | 38 | 0.681 | 4.472 | −2.392 | 1.00 | 24.19 | C |
| ATOM | 168 | CA | GLY A | 39 | −0.199 | 4.783 | −6.071 | 1.00 | 15.35 | C |
| ATOM | 169 | CA | GLY A | 40 | 0.260 | 7.491 | −8.737 | 1.00 | 12.64 | C |
| ATOM | 170 | CA | GLY A | 41 | −2.766 | 9.641 | −9.587 | 1.00 | 9.24 | C |
| ATOM | 171 | CA | GLY A | 43 | −3.890 | 4.861 | −12.131 | 1.00 | 14.44 | C |
| ATOM | 172 | CA | GLY A | 44 | −5.807 | 1.735 | −11.160 | 1.00 | 21.52 | C |
| ATOM | 173 | CA | GLY A | 45 | −5.444 | 0.202 | −7.726 | 1.00 | 22.48 | C |
| ATOM | 174 | CA | GLY A | 46 | −6.964 | −2.720 | −5.861 | 1.00 | 21.22 | C |
| ATOM | 175 | CA | GLY A | 47 | −7.458 | −2.391 | −2.118 | 1.00 | 20.06 | C |
| ATOM | 176 | CA | GLY A | 48 | −7.106 | −5.988 | −0.927 | 1.00 | 13.56 | C |
| ATOM | 177 | CA | GLY A | 49 | −9.118 | −7.626 | 1.851 | 1.00 | 19.14 | C |
| ATOM | 178 | CA | GLY A | 50 | −8.738 | −11.362 | 2.401 | 1.00 | 22.49 | C |
| ATOM | 179 | CA | GLY A | 51 | −10.667 | −13.442 | 4.932 | 1.00 | 20.58 | C |
| ATOM | 180 | CA | GLY A | 52 | −11.032 | −17.051 | 6.076 | 1.00 | 24.55 | C |
| ATOM | 181 | CA | GLY A | 53 | −13.512 | −18.935 | 8.234 | 1.00 | 17.82 | C |
| ATOM | 182 | CA | GLY A | 57 | −19.932 | −13.290 | 4.102 | 1.00 | 10.39 | C |
| ATOM | 183 | CA | GLY A | 58 | −21.330 | −9.790 | 3.738 | 1.00 | 8.00 | C |
| ATOM | 184 | CA | GLY A | 59 | −18.524 | −7.465 | 2.656 | 1.00 | 7.62 | C |
| ATOM | 185 | CA | GLY A | 60 | −18.773 | −3.717 | 3.244 | 1.00 | 6.84 | C |
| ATOM | 186 | CA | GLY A | 61 | −16.384 | −0.804 | 2.777 | 1.00 | 8.48 | C |
| ATOM | 187 | CA | GLY A | 62 | −16.301 | 2.656 | 4.292 | 1.00 | 13.92 | C |
| ATOM | 188 | CA | GLY A | 63 | −14.245 | 5.726 | 3.449 | 1.00 | 11.89 | C |
| ATOM | 189 | CA | GLY A | 64 | −13.094 | 8.083 | 6.189 | 1.00 | 23.18 | C |
| ATOM | 190 | CA | GLY A | 68 | −16.689 | 12.612 | 6.413 | 1.00 | 46.37 | C |
| ATOM | 191 | CA | GLY A | 69 | −17.861 | 8.988 | 6.532 | 1.00 | 32.33 | C |
| ATOM | 192 | CA | GLY A | 70 | −19.096 | 7.427 | 3.276 | 1.00 | 18.88 | C |
| ATOM | 193 | CA | GLY A | 71 | −19.976 | 3.841 | 2.373 | 1.00 | 12.11 | C |
| ATOM | 194 | CA | GLY A | 72 | −18.208 | 2.531 | −0.728 | 1.00 | 9.54 | C |
| ATOM | 195 | CA | GLY A | 73 | −19.659 | −0.955 | −0.492 | 1.00 | 9.17 | C |
| ATOM | 196 | CA | GLY A | 77 | −22.346 | −4.092 | −3.568 | 1.00 | 17.96 | C |
| ATOM | 197 | CA | GLY A | 78 | −20.630 | −0.882 | −4.664 | 1.00 | 11.41 | C |
| ATOM | 198 | CA | GLY A | 79 | −22.720 | 2.163 | −3.704 | 1.00 | 8.01 | C |
| ATOM | 199 | CA | GLY A | 85 | −15.239 | 3.577 | −11.142 | 1.00 | 17.93 | C |
| ATOM | 200 | CA | GLY A | 86 | −15.128 | 0.968 | −8.358 | 1.00 | 15.44 | C |
| ATOM | 201 | CA | GLY A | 87 | −15.569 | −2.740 | −9.020 | 1.00 | 16.79 | C |
| ATOM | 202 | CA | GLY A | 88 | −16.272 | −5.311 | −6.312 | 1.00 | 14.07 | C |
| ATOM | 203 | CA | GLY A | 89 | −14.727 | −8.756 | −5.888 | 1.00 | 16.75 | C |
| ATOM | 204 | CA | GLY A | 91 | −10.820 | −10.288 | −2.524 | 1.00 | 17.13 | C |
| ATOM | 205 | CA | GLY A | 92 | −11.033 | −6.489 | −2.552 | 1.00 | 12.84 | C |
| ATOM | 206 | CA | GLY A | 93 | −12.232 | −3.404 | −4.409 | 1.00 | 12.33 | C |
| ATOM | 207 | CA | GLY A | 94 | −10.610 | −2.146 | −7.602 | 1.00 | 12 .43 | C |
| ATOM | 208 | CA | GLY A | 95 | −10.518 | 1.519 | −8.590 | 1.00 | 11.12 | C |
| ATOM | 209 | CA | GLY A | 96 | −10.279 | 1.926 | −12.355 | 1.00 | 16.95 | C |
| ATOM | 210 | CA | GLY A | 97 | −8.644 | 5.292 | −11.532 | 1.00 | 21.11 | C |
| ATOM | 211 | CA | GLY A | 99 | −7.443 | 11.068 | −7.844 | 1.00 | 22.37 | C |
| ATOM | 212 | CA | GLY A | 100 | −5.937 | 13.065 | −4.977 | 1.00 | 23.75 | C |
| ATOM | 213 | CA | GLY A | 101 | −9.515 | 13.338 | −3.639 | 1.00 | 22.84 | C |
| ATOM | 214 | CA | GLY A | 102 | −9.383 | 9.634 | −2.783 | 1.00 | 15.51 | C |
| ATOM | 215 | CA | GLY A | 103 | −6.718 | 10.015 | −0.070 | 1.00 | 11.57 | C |
| ATOM | 216 | CA | GLY A | 104 | −7.876 | 8.577 | 3.237 | 1.00 | 9.52 | C |
| ATOM | 217 | CA | GLY A | 105 | −8.530 | 5.333 | 5.050 | 1.00 | 12.64 | C |
| ATOM | 218 | CA | GLY A | 106 | −10.727 | 2.534 | 3.753 | 1.00 | 7.32 | C |
| ATOM | 219 | CA | GLY A | 107 | −12.073 | −0.044 | 6.175 | 1.00 | 7.52 | C |
| ATOM | 220 | CA | GLY A | 108 | −13.078 | −3.483 | 4.952 | 1.00 | 9.97 | C |
| ATOM | 221 | CA | GLY A | 109 | −15.819 | −4.936 | 7.140 | 1.00 | 12.91 | C |
| ATOM | 222 | CA | GLY A | 110 | −16.559 | −8.639 | 6.792 | 1.00 | 11.27 | C |
| ATOM | 223 | CA | GLY A | 118 | −17.046 | −10.415 | 12.491 | 1.00 | 13.29 | C |
| ATOM | 224 | CA | GLY A | 119 | −13.800 | −8.756 | 11.482 | 1.00 | 15.55 | C |
| ATOM | 225 | CA | GLY A | 120 | −12.342 | −5.613 | 9.917 | 1.00 | 11.36 | C |
| ATOM | 226 | CA | GLY A | 121 | −9.139 | −4.141 | 8.532 | 1.00 | 11.36 | C |
| ATOM | 227 | CA | GLY A | 122 | −8.151 | −0.571 | 7.641 | 1.00 | 11.14 | C |
| ATOM | 228 | CA | GLY A | 123 | −6.080 | 0.520 | 4.643 | 1.00 | 11.58 | C |
| ATOM | 229 | CA | GLY A | 124 | −4.595 | 3.978 | 4.206 | 1.00 | 15.52 | C |
| ATOM | 230 | CA | GLY A | 125 | −4.504 | 5.200 | 0.621 | 1.00 | 9.61 | C |
| ATOM | 231 | CA | GLY A | 126 | −2.079 | 7.899 | −0.493 | 1.00 | 10.84 | C |
| ATOM | 232 | CA | GLY A | 127 | −2.415 | 9.054 | −4.098 | 1.00 | 10.58 | C |
| ATOM | 233 | CA | GLY A | 128 | 0.985 | 10.216 | −5.373 | 1.00 | 14.41 | C |
| ATOM | 234 | CA | GLY A | 129 | 1.308 | 13.675 | −6.915 | 1.00 | 12.32 | C |
| TER | | | | | | | | | | |
| 1DQT | | | | | | | | | | |
| ATOM | 235 | CA | GLY C | 2 | −10.005 | −8.876 | 13.603 | 1.00 | 35.96 | C |
| ATOM | 236 | CA | GLY C | 3 | −10.267 | −7.502 | 10.101 | 1.00 | 30.20 | C |
| ATOM | 237 | CA | GLY C | 4 | −7.171 | −6.498 | 8.221 | 1.00 | 27.24 | C |
| ATOM | 238 | CA | GLY C | 5 | −6.397 | −5.009 | 4.845 | 1.00 | 23.16 | C |
| ATOM | 239 | CA | GLY C | 6 | −3.219 | −3.760 | 3.070 | 1.00 | 22.73 | C |
| ATOM | 240 | CA | GLY C | 7 | −1.859 | −0.343 | 3.998 | 1.00 | 24.33 | C |
| ATOM | 241 | CA | GLY C | 8 | −1.267 | 0.851 | 0.436 | 1.00 | 21.64 | C |
| ATOM | 242 | CA | GLY C | 9 | −2.613 | 0.109 | −3.024 | 1.00 | 20.25 | C |
| ATOM | 243 | CA | GLY C | 10 | −1.486 | 1.813 | −6.246 | 1.00 | 20.37 | C |
| ATOM | 244 | CA | GLY C | 11 | −4.559 | 2.055 | −8.480 | 1.00 | 22.46 | C |
| ATOM | 245 | CA | GLY C | 12 | −4.228 | 1.091 | −12.139 | 1.00 | 24.30 | C |
| ATOM | 246 | CA | GLY C | 14 | −7.812 | 2.779 | −15.613 | 1.00 | 30.82 | C |
| ATOM | 247 | CA | GLY C | 15 | −8.831 | 3.617 | −12.060 | 1.00 | 25.29 | C |
| ATOM | 248 | CA | GLY C | 16 | −8.949 | 0.054 | −10.709 | 1.00 | 21.63 | C |
| ATOM | 249 | CA | GLY C | 17 | −7.920 | −0.828 | −7.174 | 1.00 | 20.22 | C |
| ATOM | 250 | CA | GLY C | 18 | −8.037 | −4.397 | −5.930 | 1.00 | 20.86 | C |
| ATOM | 251 | CA | GLY C | 19 | −7.062 | −5.746 | −2.558 | 1.00 | 21.25 | C |
| ATOM | 252 | CA | GLY C | 20 | −7.903 | −8.418 | 0.003 | 1.00 | 21.73 | C |
| ATOM | 253 | CA | GLY C | 21 | −9.906 | −7.860 | 3.174 | 1.00 | 22.97 | C |
| ATOM | 254 | CA | GLY C | 22 | −9.210 | −10.573 | 5.688 | 1.00 | 26.59 | C |
| ATOM | 255 | CA | GLY C | 23 | −10.945 | −11.564 | 8.878 | 1.00 | 26.47 | C |
| ATOM | 256 | CA | GLY C | 24 | −10.701 | −13.907 | 11.826 | 1.00 | 31.54 | C |
| ATOM | 257 | CA | GLY C | 25 | −11.932 | −16.084 | 13.335 | 1.00 | 31.33 | C |
| ATOM | 258 | CA | GLY C | 32 | −20.611 | −12.339 | 5.419 | 1.00 | 21.25 | C |
| ATOM | 259 | CA | GLY C | 33 | −21.785 | −8.834 | 4.607 | 1.00 | 21.86 | C |
| ATOM | 260 | CA | GLY C | 34 | −18.854 | −6.717 | 3.456 | 1.00 | 19.78 | C |
| ATOM | 261 | CA | GLY C | 35 | −18.920 | −2.932 | 3.364 | 1.00 | 19.11 | C |
| ATOM | 262 | CA | GLY C | 36 | −16.430 | −0.515 | 1.806 | 1.00 | 19.23 | C |
| ATOM | 263 | CA | GLY C | 37 | −16.229 | 2.889 | 3.460 | 1.00 | 25.95 | C |
| ATOM | 264 | CA | GLY C | 38 | −14.212 | 5.923 | 2.415 | 1.00 | 30.90 | C |
| ATOM | 265 | CA | GLY C | 39 | −12.916 | 7.802 | 5.426 | 1.00 | 40.84 | C |
| ATOM | 266 | CA | GLY C | 43 | −16.713 | 11.053 | 8.779 | 1.00 | 46.74 | C |
| ATOM | 267 | CA | GLY C | 44 | −17.290 | 8.066 | 6.505 | 1.00 | 36.18 | C |
| ATOM | 268 | CA | GLY C | 45 | −19.030 | 7.541 | 3.173 | 1.00 | 28.21 | C |
| ATOM | 269 | CA | GLY C | 46 | −20.279 | 4.093 | 2.143 | 1.00 | 25.48 | C |
| ATOM | 270 | CA | GLY C | 47 | −18.891 | 3.200 | −1.269 | 1.00 | 23.13 | C |
| ATOM | 271 | CA | GLY C | 48 | −20.541 | −0.174 | −1.750 | 1.00 | 20.54 | C |
| ATOM | 272 | CA | GLY C | 58 | −19.057 | −12.667 | −7.642 | 1.00 | 24.08 | C |
| ATOM | 273 | CA | GLY C | 59 | −20.627 | −9.992 | −5.444 | 1.00 | 22.25 | C |
| ATOM | 274 | CA | GLY C | 60 | −21.579 | −6.311 | −5.553 | 1.00 | 22.96 | C |
| ATOM | 275 | CA | GLY C | 61 | −23.676 | −7.234 | −8.605 | 1.00 | 28.83 | C |
| ATOM | 276 | CA | GLY C | 62 | −25.740 | −4.088 | −8.210 | 1.00 | 32.00 | C |
| ATOM | 277 | CA | GLY C | 63 | −22.747 | −1.772 | −7.854 | 1.00 | 30.87 | C |
| ATOM | 278 | CA | GLY C | 66 | −17.371 | −2.468 | −7.103 | 1.00 | 23.17 | C |
| ATOM | 279 | CA | GLY C | 67 | −16.897 | −6.161 | −7.488 | 1.00 | 22.56 | C |
| ATOM | 280 | CA | GLY C | 68 | −15.405 | −9.022 | −5.517 | 1.00 | 21.60 | C |
| ATOM | 281 | CA | GLY C | 69 | −15.312 | −12.649 | −4.466 | 1.00 | 22.69 | C |
| ATOM | 282 | CA | GLY C | 75 | −12.905 | −11.173 | 0.577 | 1.00 | 23.03 | C |
| ATOM | 283 | CA | GLY C | 76 | −11.171 | −10.003 | −2.613 | 1.00 | 24.30 | C |
| ATOM | 284 | CA | GLY C | 77 | −12.370 | −6.459 | −3.308 | 1.00 | 23.46 | C |
| ATOM | 285 | CA | GLY C | 78 | −12.157 | −4.457 | −6.510 | 1.00 | 25.74 | C |
| ATOM | 286 | CA | GLY C | 79 | −13.139 | −0.780 | −6.670 | 1.00 | 26.08 | C |
| ATOM | 287 | CA | GLY C | 80 | −13.383 | 0.660 | −10.196 | 1.00 | 29.15 | C |
| ATOM | 288 | CA | GLY C | 81 | −13.950 | 3.973 | −11.951 | 1.00 | 26.23 | C |
| ATOM | 289 | CA | GLY C | 84 | −7.281 | 11.072 | −8.931 | 1.00 | 24.79 | C |
| ATOM | 290 | CA | GLY C | 85 | −9.649 | 12.909 | −6.605 | 1.00 | 25.37 | C |
| ATOM | 291 | CA | GLY C | 86 | −10.734 | 9.536 | −5.170 | 1.00 | 25.21 | C |
| ATOM | 292 | CA | GLY C | 87 | −7.287 | 9.058 | −3.657 | 1.00 | 23.95 | C |
| ATOM | 293 | CA | GLY C | 88 | −7.874 | 8.354 | 0.014 | 1.00 | 23.67 | C |
| ATOM | 294 | CA | GLY C | 89 | −8.483 | 5.896 | 2.834 | 1.00 | 22.75 | C |
| ATOM | 295 | CA | GLY C | 90 | −10.866 | 2.985 | 2.312 | 1.00 | 23.39 | C |
| ATOM | 296 | CA | GLY C | 91 | −12.127 | 0.902 | 5.210 | 1.00 | 22.55 | C |
| ATOM | 297 | CA | GLY C | 92 | −13.188 | −2.683 | 4.810 | 1.00 | 22.29 | C |
| ATOM | 298 | CA | GLY C | 93 | −15.931 | −3.797 | 7.205 | 1.00 | 20.45 | C |
| ATOM | 299 | CA | GLY C | 94 | −16.933 | −7.418 | 7.719 | 1.00 | 20.73 | C |
| ATOM | 300 | CA | GLY C | 105 | −15.090 | −5.968 | 12.589 | 1.00 | 24.94 | C |
| ATOM | 301 | CA | GLY C | 106 | −13.327 | −3.332 | 10.512 | 1.00 | 25.24 | C |
| ATOM | 302 | CA | GLY C | 107 | −9.792 | −2.872 | 9.205 | 1.00 | 24.05 | C |
| ATOM | 303 | CA | GLY C | 108 | −7.671 | 0.283 | 9.656 | 1.00 | 25.85 | C |
| ATOM | 304 | CA | GLY C | 109 | −8.117 | 1.175 | 6.019 | 1.00 | 23.77 | C |
| ATOM | 305 | CA | GLY C | 110 | −6.209 | 0.891 | 2.770 | 1.00 | 22.46 | C |
| ATOM | 306 | CA | GLY C | 111 | −4.626 | 4.045 | 1.330 | 1.00 | 24.13 | C |
| ATOM | 307 | CA | GLY C | 112 | −5.545 | 4.027 | −2.339 | 1.00 | 22.83 | C |
| ATOM | 308 | CA | GLY C | 113 | −3.438 | 6.352 | −4.496 | 1.00 | 23.40 | C |
| ATOM | 309 | CA | GLY C | 114 | −4.906 | 7.221 | −7.840 | 1.00 | 25.42 | C |
| END | | | | | | | | | | |

TABLE 2

Exemplary amino acid sequences likely to fold as
nine stranded iMab proteins

iMab100
NVKLVE--KGG-NFVEN--DDDL--KLTCRAEGYTI----GPYCMGWFRQ
APNDDSTNVATINMGGGITYYGDSVKERFDIRRDNASNTVTLSMDDLQP
ED---SAEYNCAGDSTIYASYYECGHGLSTGGYGYDSHYR--GQ-GTDVT
VSSA
iMab502
SVKFVC--KVLPNFWEN--NKDLPTKFTVRASGYTI----GPTCVGVFAQ
NPEDDSTNVATINMGGGITYYGDSVKLRFDIRRDNAKVTRTNSLDDVQP
EGRGKSFELTCAADSTIYASYYECGHGISTGGYGYDQVAR--YHRGIDIT
VDGP
iMab702
AVKSVF--KVSTNFIENDGTMDS--KLTFRASGYTI----GPOCLGFFQQ
GVPDDSTNVATINMGGGITYYGDSVKSIFDIRRDNAKDTYTASVDDNQP
E----DVEITCAADSTIYASYYECGHGISTGGYGYDLILRTLQK-GIDLF
VVPT
iMab1202 (1EJ6)
IVKLVM--EKR-GNFEN--GQDC--KLTIRASGYTI----GPACDGFFCQ
FPSDDSFSTED-NMGGGIT-VNDAMKPQFDIRRDNAKGTWTLSM-DFQP
EG---IYEMQCAADSTIYASYYECGHGISTGGYGYDNPVR--LG-GFDVD
VPDV
iMab1302
VVKVVI--KPSQNFIEN--GEDK--KFTCRASGYTI----GPKCIGWFSQ
NPEDDSTNVATINMGGGITYYGDSVKERFDIRRDNAKDTSTLSIDDAQP
ED---AGIYKCAADSTIYASYYECGHGISTGGYGYDSEA---TV-GVDIF
VKLM
iMab1502 (1NEU)
NVKVVT--KRE-NFGEN--GSDV--KLTCRASGYTI----GPICFGWFYQ
PEGDDSAISIFHNMGGGITDEVDTFKERFDIRRDNAKKTGTISIDDLQP
SD---NETFTCAADSTIYASYYECGHGISTGGYGYDGKTR--QV-GLDVF
VKVP
iMab1602
AVKPVIGSKAP-NFGEN---GDV--KTIDRASGYTI----GPTCGGVFFQ
GPTDDSTNVATINMGGGITYYGDSVKETFDIRRDNAKSTRTESYDDNQP
EG---LTEVKCAADSTIYASYYECGHGISTGGYGYDVSSR--LY-GYDIL
VGTQ

TABLE 3
VAP amino acid sequences

iMab100
NVKLVEKGGNFVENDDDLKLTCRAEGYTIGPYCMGWFRQAPNDDSTNVATINMGGGITY
YGDSVKER
FDIRRDNASNTVTLSMDDLQPEDSAEYNCAGDSTIYASYYECGHGLSTGGYGYDSHYRG
QGTDVTVSS
iMab101
VKLVEKGGNFVENDDDLKLTCRASGYTIGPYCMGWFRQAPNDDSTNVATINMGTVTLS
MDDLQPEDSA
EYNCAADSTIYASYYECGHGLSTGGYGYDSHYRGQGTDVTVSS
iMab102
DLKLTCRASGYTIGPYCMGWFRQAPNDDSTNVATINMGTVTLSMDDLQPEDSAEYNCAA
DSTIYASYY
ECGHGLSTGGYGYDSHYRGQGTDVTVSS
iMab111
NVKLVCKGGNFVENDDDLKLTCRAEGYTIGPYCMGWFRQAPNDDSTNVATINMGGGIT
YYGDSVKER
FDIRRDNASNTVTLSMDDLQPEDSAEYNCAGDSTIYASYYECGHGLSTGGYGYDSHYRC
QGTDVTVSS
iMab112
NVKLVEKGGNFVENDDDLKLTCRAEGYTIGPYCMGWFCQAPNDDSTCVATINMGGGITY
YGDSVKER
FDIRRDNASNTVTLSMDDLQPEDSAEYNCAGDSTIYASYYECGHGLSTGGYGYDSHYRG
QGTDVTVSS
iMab113
NVKLVEKGGNFVENDDDLKLTCRAEGYTIGPYSMGWFRQAPNDDSTNVSCINMGGGITY
YGDSVKER
FDIRRDNASNTVTLSMDDLQPEDSAEYNCAGDSTIYASYYECGHGLSTGGYGYDSHYRG
QGTDVTVSS
iMab114
NVKLVEKGGNFVENDDDLKLTCRAEGYTIGPYSMGWFRQAPNDDSTNVATINMGGGITY
YGDSVKER
FDIRRDNASNTVTLSMDDLQPEDSAEYNCAGDSTIYASYYECGHGLSTGGYGYDSHYRG
QGTDVTVSS
iMab115
NVKLVEKGGNFVENDDDLKLTCRAEGYTIGPYCMGWFRQAPNDDSTNVATINMGGGITY
YGDSVKER
FDIRRDQASNTVTLSMDDLQPEDSAEYNCAGDSTIYASYYECGHGLSTGGYGYDSHYRG
QGTDVTVSS
iMab116
NVKLVEKGGNFVENPDDLKLTCRAEGYTIGPYCMGWFRQAPNDDSTNVATINMGGGITY
YGDSVKER
FDIRRDNASNTVTLSMDDLQPEDSAEYNGAGDSTIYGSYYECGHGLSTGGYGYDSHYRG
QGTDVTVSS
iMab120
NVKLVEKGGNFVENDDDLKLTCRAEGYTIGPYCMGWFRQAPNDDSTNVATINMGGGITY
YGDSVKER
FDIRRDNASNTVTLSMDDLQPEDSAEYNCAGDSTIYASYYECGHGLSTGGYGYDSRGQGT
DVTVSS
iMab121
NVKLVEKGGNFVENDDDLKLTCRASGRSFSSYTMGWFRQAPNDDSTNVATISETGGDIVY
TNYGDSVKER
FDIRRDIASNTVTLSMDDLQPEDSAEYNCAGSVYGSGWRPDRYDYRGQGTDVTVSS
iMab124
DDLKLTCRASGRSFSSYIMGWFRQAPNDDSTNVATISETTVTLSMDDLQPEDSAEYNCAG
SVYGSGWRPD
RYDYRGQGTDVTVSS
iMab122
NVKLVEKGGNFVENDDDLKLTCRASGRTFSSRTMGWFRQAPNDDSTNVATIRWNGGST
YYTNYGDSVKER
FDIRVDQASNTVTLSMDDLQPEDSAEYNCAGTDIGDGWSGRYDYRGQGTDVTVSS
iMab125
DDLKLTCRASGRTFSSRTMGWFRQAPNDDSThVATIRWNTVTLSMDDLQPEDSAEYNCA
GTDIGDGWSGR
YDYRGQGTDVTVSS
iMab123
NVKLVEKGGNFVENDDDLKLTCRASGRTFSRAAMGWFRQAPNDDSTNVATITWSGHT
RYGDSVKER
FDIRRDQASNTVTLSMDDLQPEDSAEYNCAGEGSNTASTSPRPYDYRGQGTDVTVSS
iMab130
NVKLVEKGGNFVENDDDLKLTCRASGYAYTYIYMGWFRQAPNDDSTNVATIDSGGGGT
LYGDSVKER
FDIRRDKGSNTVTLSMDDLQPEDSAEYNCAAGGYELRDRTYGQRGQGTDVTVSS
iMab201
VQLQASGGGSVQAGGSLRLSCRASGYTIGPYCMGWFRQAPGDDSEGVAAINMGTVYLL
MNSLEPEDT
AIYYCAADSTIYASYYECGHGLSTGGYGYDSWGQGTQVTVSS
iMab300
VQLQQPGSNLVRPGASVKLSCKASGYTIGPSCIHWAKQRPGDGLEWIGEINMGTAYVDLS
SLTSEDS
AVYYCAADSTIYASYYECGHGLSTGGYGYDYWGQGTTLTVSS
iMab302
ASVKLSCKASGYTIGPSCIHWAKQRPGDGLEWIGEINMGTAYVDLSSLTSEDSAVYYCAA
DSTIYAS
YYECGHGLSTGGYGYDYWGQGTTLTVSS
iMab400
VQLVESGGGLVQPGGSLRLSCRASGYTIGPYCMNWVRQAPGDGLEWVGWINMGTAYLQ
MNSLRAEDT
AVYYCAADSTIYASYYECGHGLSTGGYGYDVWGQGTLVTVSS
iMab500
PNFLCSVLPTHWRCNKTLPIAFKCRASGYTIGPTCVTVMAGNDEDYSNMGARFNDLRFVG
RSGRGKS
FTLTCAADSTIYASYYECGHGLSTGGYGYPQVATYHRAIKITVDGP
iMab502
SVKFVCKVLPNFWENNKDLPIKFTVRASGYTIGPTCVGVFAQNPEDDSTNVATINMGGGI
TYYGDSVKLR
FDIRRDNAKVTRTNSLDDVQPEGRGKSFELTCAADSTIYASYYECGHGLSTGGYGYDQVA
RYHRGIDITVDGP
iMab600
APVGLKARNADESGHVVLRCRASGYTIGPICYEVDVSAGQDAGSVQRVEINMGRTESVLS
NLRGRTRYTFA
CAADSTIYASYYECGHGLSTGGYGYSEWSEPVSLLTPS
iMab700
DKSTLAAVPTSIIADGLMASTITCEASGYTIGPACVAFDTFFLGNNMGTYSAPLTSTTLGVA
TVTCANDST
IYASYYECGHGLSTGGYGYAAFSVPSVTVNFTA
iMab702
AVKSVFKVSTNFIENDGTMDSKLTFRASGYTIGPQCLGFFQQGVPDDSTNVATINMGGGIT
YYGDSVKSI
FDIRRDNAKDTYTASVDDNQPEDVEITCAADSTIYASYYECGHGLSTGGYGYDLILRTLQ
KGIDLFVVPT
iMab701
MASTITCEASGYTIGPACVAFDTTLGNNMGTYSAPLTSTTLGVATVTCAADSTIYASYYEC
GHGLSTGGY
GYAAFSVPSVTVNFTA
iMab800
GRSSFTVSTPDILADGTMSSTLSCRASGYTIGPQCLSFTQNGVPVSISPINMGSYTATVVGN
SVGDVTITC
AADSTIYASYYECGHGLSTGGYGYTLILSTLQKKISLFP
iMab900
LTLTAAVIGDGAPANGKTAITVECTASGYTIGPQCVVITTNNGALPNKITENMGVARIALT
NTTDGVTVVT
CAADSTIYASYYECGHGLSTGGYGYQRQSVDTHFVK
iMab1000
HKPVIEKVDGGYLCKASGYTIGPECIELLADGRSYTKNMGEAFFAIDASKVTCAADSTIYA
SYYECGHGLS
TGGYGYHWKAEN
iMab1001
VDGGYLCKASGYTIGPECIELLADGRSYTKNMGEAFFAIDASKVTCAADSTIYASYYECG
HGLSTGGYGYHWKAEN
iMab1100
APVGLKARLADESGHVVLRCRASGYTIGPICYEVDVSAGNDAGSVQRVEILNMGTESVLS
NLRGRTRYTFACA
ADSTIYASYYECGHGLSTGGYGYSAWSEPVSLLTPS
iMab1200
HGLPMEKRGNFIVGQNCSLTCPASGYTIGPQCVFNCYFNSALAFSTENMGEWTLDMVFSD
AGIYTMCAADS
TIYASYYECGHGLSTGGYGYNPVSLGSFVVDSP
iMab1202
IVKLVMEKRGNFENGQDCKLTIRASGYTIGPACDGFFCQFPSDDSFSTEDNMGGGITVNDA
MKPQFDIRRDNAKGTWTLSMDFQPEGIYEMQCAADSTIYASYYECGHGLSTGGYGYDNP
VRLGGFDVDVPDV
iMab1300
LQVDIKPSQGEISVGESKFFLCQASGYTIGPCISWFSPNGEKLNMGSSTLTIYNANIDDAGIY
KCAADSTIY
ASYYECGHGLSTGGYGYQSEATVNVKIFQ
iMab1302
VVKVVIKPSQNFIENGEDKKFTCRASGYTIGPKCIGWFSQNPEDDSTNVATINMGGGITYY
GDSVKER
FDIRRDNAKDTSTLSIDDAQPEDAGIYKCAADSTIYASYYECGHGLSTGGYGYDSEATVG
VDTFVKLM
iMab1301
ESKFFLCQASGYTIGPCISWFSPNGEKLNMGSSTLTIYNANIDDAGIYKCAADSTIYASYYE
CGHGLS
TGGYGYQSEATVNVKIFQ
iMab1400
VPRDLEVVAATPTSLLISCDASGYTIGPYCITYGETGGNSPVQEFTVPNMG
KSTATISGLKPGVDYTITCAADSTIYASYYECGHGLSTGGYGYSKPISINYRT
iMab1500
IKVYTDRENYGAVGSQVTLHCSASGYTIGPICFTWRYQPEGDRDAISTFHYNMGDGSIVIH
NLDYS
DNGTFTCAADSTIYASYYECGHGLSTGGYGYVGKTSQVTLYVFE
iMab1502
NVKVVTKRENFGENGSDVKLTCRASGYTIGPICFGWFYQPEGDDSAISIFHNMGGGITDEV
DTFKER
FDIRRDNAKKTGTISIDDLQPSDNETFTCAADSTIYASYYECGHGLSTGGYGYDGKTRQVG
LDVFVKVP
iMab1501
SQVTLHCSASGYTIGPICFTWRYQPEGDRDAISIFHYNMGDGSIVIHNLDYSDNGTFTCAA
DSTIYAS
YYECGHGISTGGYGYVGKTSQVTLYVFE
iMab1600
SKPQIGSVAPNMGIPGNDVTITCRASGYTIGPTCGTVTFGGVTNMGNRIEVYVPNMAAGL
TDVKCAA
DSTIYASYYECGHGLSTGGYGYGVSSNLYSYNILS
iMab1602
AVKPVIGSKAPNFGENGDVKTIDRASGYTIGPTCGGVFFQGPTDDSTNVATINMGGGITYY
GDSVKET
FDIRRDNAKSTRTESYDDNQPEGLTEVKCAADSTIYASYYECGHGLSTGGYGYDVSSRLY
GYDILVGTQ
iMab1700
KDPEIHLSGPLEAGKPITVKCSASGYTIGPLCIDLLKGDHLMKSQEFNMGSLEVTFTPVIEDI
GKVLVC
AADSTIYASYYECGHGLSTGGYGYVRQAVKELQVD
iMab1701
KPITVKCSASGYTIGPLCIDLLKGDHLMKSQEFNMGSLEVTFTPVIEDIGKVLVCAADSTIY
ASYYEC
GHGLSTGGYGYVRQAVKELQVD

TABLE 4
iMab DNA sequences

| | |
|---|---|
| iMab D100 | |
| 1 | AATGTGAAAC TGGTTGAAAA AGGTGGCAAT TTCGTCGAAA ACGATGACGA |
| | TCTTAAGCTC ACGTGCCGTG CTGAAGGTTA |
| 81 | CACCATTGGC CCGTACTGCA TGGGTTGGTT CCGTCAGGCG CCGAACGACG |
| | ACAGTACTAA CGTGGCCACG ATCAACATGG |
| 161 | GTGGCGGTAT TACGTACTAC GGTGACTCCG TCAAAGAGCG CTTCGATATC |
| | CGTCGCGACA ACGCGTCCAA CACCGTTACC |
| 241 | TTATCGATGG ACGATCTGCA ACCGGAAGAC TCTGCAGAAT ACAATTGTGC |
| | AGGTGATTCT ACCATTTACG CGAGCTATTA |
| 321 | TGAATGTGGT CATGGCCTGA GTACCGGCGG TTACGGCTAC GATAGCCACT |
| | ACCGTGGTCA GGGTACCGAC GTTACCGTCT |
| 401 | CG |
| iMab D101 | |
| 1 | CATA TGGTTAAACT GGTTGAAAAA GGTGGTAACT TCGTTGAAAA |
| | CGACGACGAC CTGAAACTGA CCTGCCGTGC |
| 81 | TTCCGGTTAC ACCATCGGTC CGTACTGCAT GGGTTGGTTC CGTCAGGCTC |
| | CGAACGACGA CTCCACCAAC GTTGCTACCA |
| 161 | TCAACATGGG TACCGTTACC CTGTCCATGG ACGACCTGCA GCCGGAAGAC |
| | TCCGCTGAAT ACAACTGCGC TGCTGACTCC |
| 241 | ACCATCTACG CTTCCTACTA CGAATGCGGT CACGGTATCT CCACCGGTGG |
| | TTACGGTTAC GACTCCCACT ACCGTGGTCA |
| 321 | GGGTACCGAC GTTACCGTTT CCTCGGCCAG CTCGGCC |
| iMab D102 | |
| 1 | CATA TGGACCTGAA ACTGACCTGC CGTGCTTCCG GTTACACCAT |
| | CGGTCCGTAC TGCATGGGTT GGTTCCGTCA |
| 81 | GGCTCCGAAC GACGACTCCA CCAACGTTGC TACCATCAAC ATGGGTACCG |
| | TTACCCTGTC CATGGACGAC CTGCAGCCGG |
| 161 | AAGACTCCGC TGAATACAAC TGCGCTGCTG ACTCCACCAT CTACGCTTCC |
| | TACTACGAAT GCGGTCACGG TATCTCCACC |
| 241 | GGTGGTTACG GTTACGACTC CCACTACCGT GGTCAGGGTA CCGACGTTAC |
| | CGTTTCCTCG GCCAGCTCGG CC |
| iMab D111 | |
| 1 | CATATG AATGTGAAAC TGGTTTGTAA AGGTGGCAAT TTCGTCGAAA |
| | ACGATGACGA TCTTAAGCTC ACGTGCCGTG |
| 81 | CTGAAGGTTA CACCATTGGC CCGTACTGCA TGGGTTGGTT CCGTCAGGCG |
| | CCGAACGACG ACAGTACTAA CGTGGCCACG |
| 161 | ATCAACATGG GTGGCGGTAT TACGTACTAC GGTGACTCCG TCAAAGAGCG |
| | CTTCGATATC CGTCGCGACA ACGCGTCCAA |
| 241 | CACCGTTACC TTATCGATGG ACGATCTGCA ACCGGAAGAC TCTGCAGAAT |
| | ACAATTGTGC AGGTGATTCT ACCATTTACG |
| 321 | CGAGCTATTA TGAATGTGGT CATGGCCTGA GTACCGGCGG TTACGGCTAC |
| | GATAGCCACT ACCGTTGCCA GGGTACCGAC |
| 401 | GTTACCGTCT CGTCGGCCAG CTCGGCC |
| iMab D112 | |
| 1 | AATGTGAAAC TGGTTGAAAA AGGTGGCAAT TTCGTCGAAA ACGATGACGA |
| | TCTTAAGCTC ACGTGCCGTG CTGAAGGTTA |
| 81 | CACCATTGGC CCGTACTGCA TGGGTTGGTT CTGTCAGGCG CCGAACGACG |
| | ACAGTACTTG CGTGGCCACG ATCAACATGG |
| 161 | GTGGCGGTAT TACGTACTAC GGTGACTCCG TCAAAGAGCG CTTCGATATC |
| | CGTCGCGACA ACGCGTCCAA CACCGTTACC |
| 241 | TTATCGATGG ACGATCTGCA ACCGGAAGAC TCTGCAGAAT ACAATTGTGC |
| | AGGTGATTCT ACCATTTACG CGAGCTATTA |
| 321 | TGAATGTGGT CATGGCCTGA GTACCGGCGG TTACGGCTAC GATAGCCACT |
| | ACCGTGGTCA GGGTACCGAC GTTACCGTCT |
| 401 | CGTCG |
| iMab D113 | |
| 1 | AATGTGAAAC TGGTTGAAAA AGGTGGCAAT TTCGTCGAAA ACGATGACGA |
| | TCTTAAGCTC ACGTGCCGTG CTGAAGGTTA |
| 81 | CACCATTGGC CCGTACTCCA TGGGTTGGTT CCGTCAGGCG CCGAACGACG |
| | ACAGTACTAA CGTGTCCTGC ATCAACATGG |
| 161 | GTGGCGGTAT TACGTACTAC GGTGACTCCG TCAAAGAGCG CTTCGATATC |
| | CGTCGCGACA ACGCGTCCAA CACCGTTACC |
| 241 | TTATCGATGG ACGATCTGCA ACCGGAAGAC TCTGCAGAAT ACAATTGTGC |
| | AGGTGATTCT ACCATTTACG CGAGCTATTA |
| 321 | TGAATGTGGT CATGGCCTGA GTACCGGCGG TTACGGCTAC GATAGCCACT |
| | ACCGTGGTCA GGGTACCGAC GTTACCGTCT |
| 401 | CGTCG |
| iMab D114 | |
| 1 | AATGTGAAAC TGGTTGAAAA AGGTGGCAAT TTCGTCGAAA ACGATGACGA |
| | TCTTAAGCTC ACGTGCCGTG CTGAAGGTTA |
| 81 | CACCATTGGC CCGTACTCCA TGGGTTGGTT CCGTCAGGCG CCGAACGACG |
| | ACAGTACTAA CGTGGCCACG ATCAACATGG |
| 161 | GTGGCGGTAT TACGTACTAC GGTGACTCCG TCAAAGAGCG CTTCGATATC |
| | CGTCGCGACA ACGCGTCCAA CACCGTTACC |
| 241 | TTATCGATGG ACGATCTGCA ACCGGAAGAC TCTGCAGAAT ACAATTGTGC |
| | AGGTGATTCT ACCATTTACG CGAGCTATTA |
| 321 | TGAATGTGGT CATGGCCTGA GTACCGGCGG TTACGGCTAC GATAGCCACT |
| | ACCGTGGTCA GGGTACCGAC GTTACCGTCT |
| 401 | CGTCG |
| iMab D115 | |
| 1 | AATGTGAAAC TGGTTGAAAA AGGTGGCAAT TTCGTCGAAA ACGATGACGA |
| | TCTTAAGCTC ACGTGCCGTG CTGAAGGTTA |
| 81 | CACCATTGGC CCGTACTGCA TGGGTTGGTT CCGTCAGGCG CCGAACGACG |
| | ACAGTACTAA CGTGGCCACG ATCAACATGG |
| 161 | GTGGCGGTAT TACGTACTAC GGTGACTCCG TCAAAGAGCG CTTCGATATC |
| | CGTCGCGACC AGGCGTCCAA CACCGTTACC |
| 241 | TTATCGATGG ACGATCTGCA ACCGGAAGAC TCTGCAGAAT ACAATTGTGC |
| | AGGTGATTCT ACCATTTACG CGAGCTATTA |
| 321 | TGAATGTGGT CATGGCCTGA GTACCGGCGG TTACGGCTAC GATAGCCACT |
| | ACCGTGGTCA GGGTACCGAC GTTACCGTCT |
| 401 | CGTCG |
| iMab D116 | |
| 1 | CATATG AATGTGAAAC TGGTTGAAAA AGGTGGCAAT TTCGTCGAAA |
| | ACGATGACGA TCTTAAGCTC ACGTGTCGTG |
| 81 | CTGAAGGTTA CACCATTGGC CCGTACTGCA TGGGTTGGTT CCGTCAGGCG |
| | CCGAACGACG ACAGTACTAA CGTGGCCACG |
| 161 | ATCAACATGG GTGGCGGTAT TACGTACTAC GGTGACTCCG TCAAAGAGCG |
| | CTTCGATATC CGTCGCGACA ACGCGTCCAA |
| 241 | CACCGTTACC TTATCGATGG ACGATCTGCA ACCGGAAGAC TCTGCAGAAT |
| | ACAATGGTGC AGGTGATTCT ACCATTTACG |
| 321 | GGAGCTATTA TGAATGTGGT CATGGCCTGA GTACCGGCGG TTACGGCTAC |
| | GATAGCCACT ACCGTGGTCA GGGTACCGAC |
| 401 | GTTACCGTCT CGTCGGCCAG CTCGGCC |
| iMab D120 | |
| 1 | AATGTGAAAC TGGTTGAAAA AGGTGGCAAT TTCGTCGAAA ACGATGACGA |
| | TCTTAAGCTC ACGTGCCGTG CTGAAGGTTA |
| 81 | CACCATTGGC CCGTACTGCA TGGGTTGGTT CCGTCAGGCG CCGAACGACG |
| | ACAGTACTAA CGTGGCCACG ATCAACATGG |
| 161 | GTGGCGGTAT TACGTACTAC GGTGACTCCG TCAAAGAGCG CTTCGATATC |
| | CGTCGCGACA ACGCGTCCAA CACCGTTACC |
| 241 | TTATCGATGG ACGATCTGCA ACCGGAAGAC TCTGCAGAAT ACAATTGTGC |
| | AGGTGATTCT ACCATTTACG CGAGCTATTA |
| 321 | TGAATGTGGT CATGGCCTGA GTACCGGCGG TACGGCTAC GATAGCCGTG |
| | GTCAGGGTAC CGACGTTACC GTCTCGTCG |
| iMab D121 | |
| 1 | CATA TGAACTTTAA ACTGGTTGAA AAAGGTGGTA ACTTCGTTGA |
| | AAACGACGAC GACCTGAAAC TGACCTGCCG |
| 81 | TGCTTCCGGT CGTTCCTTCT CCTCCTACAT CATGGGTTGG TTCCGTCAGG |
| | CTCCGAACGA CGACTCCACC AACGTTGCTA |
| 161 | CCATCTCCGA AACCGGTGGT GACATCGTTT ACACCAACTA CGGTGACTCC |
| | GTTAAAGAAC GTTTCGACAT CCGTCGTGAC |
| 241 | ATCGCTTCCA ACACCGTTAC CCTGTCCATG GACGACCTGC AGCCGGAAGA |
| | CTCCGCTGAA TACAACTGCG CTGGTTCCGT |
| 321 | TTACGGTTCC GGTTGGCGTC CGGACCGTTA CGACTACCGT GGTCAGGGTA |
| | CCGACGTTAC CGTTTCCTCG GCCAGCTCGG |
| 401 | CC |
| iMab D122 | |
| 1 | CATA TGAACGTTAA ACTGGTTGAA AAAGGTGGTA ACTTCGTTGA |
| | AAACGACGAC GACCTGAAAC TGACCTGCCG |
| 81 | TGCTTCCGGT CGTACCTTCT CCTCCCGTAC CATGGGTTGG TTCCGTCAGG |
| | CTCCGAACGA CGACTCCACC AACGTTGCTA |
| 161 | CCATCCGTTG GAACGGTGGT TCCACCTACT ACACCAACTA CGGTGACTCC |
| | GTTAAAGAAC GTTTCGACAT CCGTGTTGAC |
| 241 | CAGGCTTCCA ACACCGTTAC CCTGTCCATG GACGACCTGC AGCCGGAAGA |
| | CTCCGCTGAA TACAACTGCG CTGGTACCGA |
| 321 | CATCGGTGAC GGTTGGTCCG GTCGTTACGA CTACCGTGGT CAGGGTACCG |
| | ACGTTACCGT TTCCTCGGCC AGCTCGGCC |
| iMab D123 | |
| 1 | CATA TGAACGTTAA ACTGGTTGAA AAAGGTGGTA ACTTCGTTGA |
| | AAACGACGAC GACCTGAAAC TGACCTGCCG |
| 81 | TGCTTCCGGT CGTACCTTCT CCCGTGCTGC TATGGGTTGG TTCCGTCAGG |
| | CTCCGAACGA CGACTCCACC AACGTTGCTA |
| 161 | CCATCACCTG GTCCGGTCGT CACACCCGTT ACGGTGACTC CGTTAAAGAA |
| | CGTTTCGACA TCCGTCGTGA CCAGGCTTCC |
| 241 | AACACCGTTA CCCTGTCCAT GGACGACCTG CAGCCGGAAG ACTCCGCTGA |
| | ATACAACTGC GCTGGTGAAG GTTCCAACAC |
| 321 | CGCTTCCACC TCCCCGCGTC CGTACGACTA CCGTGGTCAG GGTACCGACG |
| | TTACCGYITC CTCGGCCAGC TCGGCC |
| iMab D124 | |
| 1 | CATA TGGACGACCT GAAACTGACC TGCCGTGCTT CCGGTCGTTC |
| | CTTCTCCTCC TACATCATGG GTTGGTTCCG |
| 81 | TCAGGCTCCG AACGACGACT CCACCAACGT TGCTACCATC TCCGAAACCA |
| | CCGTTACCCT GTCCATGGAC GACCTGCAGC |
| 161 | CGGAAGACTC CGCTGAATAC AACTGCGCTG GTTCCGTTTA CGGTTCCGGT |
| | TGGCGTCCGG ACCGTTACGA CTACCGTGGT |
| 241 | CAGGGTACCG ACGTTACCGT TTCCTCGGCC AGCTCGGCC |
| iMab D125 | |
| 1 | CATA TGGACGACCT GAAACTGACC TGCCGTGCTT CCGGTCGTAC |
| | CTTCTCCTCC CGTACCATGG GTTGGTTCCG |
| 81 | TCAGGCTCCG AACGACGACT CCACCAACGT TGCTACCATC CGTTGGAACA |
| | CCGTTACCCT GTCCATGGAC GACCTGCAGC |
| 161 | CGGAAGACTC CGCTGAATAC AACTGCGCTG GTACCGACAT CGGTGACGGT |
| | TGGTCCGGTC GTTACGACTA CCGTGGTCAG |
| 241 | GGTACCGACG TTACCGTTTC CTCGGCCAGC TCGGCC |
| iMab D130 | |
| 1 | A ATGTGAAACT GGTTGAAAAA GGTGGCAATT TCGTCGAAAA |
| | CGATGACGAT CTTAAGCTCA CGTGCCGTGC |
| 81 | TAGCGGTTAC GCCTACACGT ATATCTACAT GGGTTGGTTC CGTCAGGCGC |
| | CGAACGACGA CAGTACTAAC GTGGCCACCA |
| 161 | TCGACTCGGG TGGCGGCGGT ACCCTGTACG GTGACTCCGT CAAAGAGCGC |
| | TTCGATATCC GTCGCGACAA AGGCTCCAAC |
| 241 | ACCGTTACCT TATCGATGGA CGATCTGCAA CCGGAAGACT CTGCAGAATA |
| | CAATTGTGCA GCGGGTGGCT ACGAACTGCG |
| 321 | CGACCGCACC TACGGTCAGC GTGGTCAGGG TACCGACGTT ACCGTCTCGT |
| | CGGCCAGCTC GGCC |
| iMab D201 | |
| 1 | CATA TGGTTCAGCT GCAGGCTTCC GGTGGTGGTT CCGTTCAGGC |
| | TGGTGGTTCC CTGCGTCTGT CCTGCCGTGC |
| 81 | TTCCGGTTAC ACCATCGGTC CGTACTGCAT GGGTTGGTTC CGTCAGGCTC |
| | CGGGTGACGA CTCCGAAGGT GTTGCTGCTA |
| 161 | TCAACATGGG TACCGTTTAC CTGCTGATGA ACTCCCTGGA ACCGGAAGAC |
| | ACCGCTATCT ACTACTGCGC TGCTGACTCC |
| 241 | ACCATCTACG CTTCCTACTA CGAATGCGGT CACGGTATCT CCACCGGTGG |
| | TTACGGTTAC GACTCCTGGG GTCAGGGTAC |
| 321 | CCAGGTTACC GTTTCCTCGG CCAGCTCGGC C |
| iMab D300 | |
| 1 | CATA TGGTTCAGCT GCAGCAGCCG GGTTCCAACC TGGTTCGTCC |
| | GGGTGCTTCC GTTAAACTGT CCTGCAAAGC |
| 81 | TTCCGGTTAC ACCATCGGTC CGTCCTGCAT CCACTGGGCT AAACAGCGTC |
| | CGGGTGACGG TCTGGAATGG ATCGGTGAAA |
| 161 | TCAACATGGG TACCGCTTAC GTTGACCTGT CCTCCCTGAC CTCCGAAGAC |
| | TCCGCTGTTT ACTACTGCGC TGCTGACTCC |
| 241 | ACCATCTACG CTTCCTACTA CGAATGCGGT CACGGTATCT CCACCGGTGG |
| | TTACGGTTAC GACTACTGGG GTCAGGGTAC |
| 321 | CACCCTGACC GTTTCCTCGG CCAGCTCGGC C |
| iMab D302 | |
| 1 | CATA TGGCTTCCGT TAAACTGTCC TGCAAAGCTT CCGGTTACAC |
| | CATCGGTCCG TCCTGCATCC ACTGGGCTAA |
| 81 | ACAGCGTCCG GGTGACGGTC TGGAATGGAT CGGTGAAATC AACATGGGTA |
| | CCGCTTACGT TGACCTGTCC TCCCTGACCT |
| 161 | CCGAAGACTC CGCTGTTTAC TACTGCGCTG CTGACTCCAC CATCTACGCT |
| | TCCTACTACG AATGCGGTCA CGGTATCTCC |
| 241 | ACCGGTGGTT ACGGTTACGA CTACTGGGGT CAGGGTACCA CCCTGACCGT |
| | TTCCTCGGCC AGCTCGGCC |
| iMab D400 | |
| 1 | CATA TGGTTCAGCT GGTTGAATCC GGTGGTGGTC TGGTTCAGCC |
| | GGGTGGTTCC CTGCGTCTGT CCTGCCGTGC |
| 81 | TTCCGGTTAC ACCATCGGTC CGTACTGCAT GAACTGGGTT CGTCAGGCTC |
| | CGGGTGACGG TCTGGAATGG GTTGGTTGGA |
| 161 | TCAACATGGG TACCGCTTAC CTGCAGATGA ACTCCCTGCG TGCTGAAGAC |
| | ACCGCTGTTT ACTACTGCGC TGCTGACTCC |
| 241 | ACCATCTACG CTTCCTACTA CGAATGCGGT CACGGTATCT CCACCGGTGG |
| | TTACGGTTAC GACGTTTGGG GTCAGGGTAC |
| 321 | CCTGGTTACC GTTTCCTCGG CCAGCTCGGC C |
| iMab D500 | |
| 1 | CATA TGCCGAACTT CCTGTGCTCC GTTCTGCCGA CCCACTGGCG |
| | TTGCAACAAA ACCCTGCCGA TCGCTTTCAA |
| 81 | ATGCCGTGCT TCCGGTTACA CCATCGGTCC GACCTGCGTT ACCGTTATGG |
| | CTGGTAACGA CGAAGACTAC TCCAACATGG |
| 161 | GTGCTCGTTT CAACGACCTG CGTTTCGTTG GTCGTTCCGG TCGTGGTAAA |
| | TCCTTCACCC TGACCTGCGC TGCTGACTCC |
| 241 | ACCATCTACG CTTCCTACTA CGAATGCGGT CACGGTATCT CCACCGGTGG |
| | TTACGGTTAC CCGCAGGTTG CTACCTACCA |
| 321 | CCGTGCTATC AAAATCACCG TTGACGGTCC GGCCAGCTCG GCC |
| iMab D502 | |
| 1 | CATA TGTCCGTTAA ATTCGTTTGC AAAGTTCTGC CGAACTTCTG |
| | GGAAAACAAC AAAGACCTGC CGATCAAATT |
| 81 | CACCGTTCGT GCTTCCGGTT ACACCATCGG TCCGACCTGC GTTGGTGTTT |
| | TCGCTCAGAA CCCGGAAGAC GACTCCACCA |
| 161 | ACGTTGCTAC CATCAACATG GGTGGTGGTA TCACCTACTA CGGTGACTCC |
| | GTTAAACTGC GTTTCGACAT CCGTCGTGAC |
| 241 | AACGCTAAAG TIACCCGTAC CAACTCCCTG GACGACGTTC AGCCGGAAGG |
| | TCGTGGTAAA TCCTTCGAAC TGACCTGCGC |
| 321 | TGCAGACTCC ACCATCTACG CTTCCTACTA CGAATGCGGT CACGGTCTGT |
| | CCACCGGTGG TTACGGTTAC GACCAGGTTG |
| 401 | CTCGTTACCA CCGTGGTATC GACATCACCG TCTCGTCGGC CAGCTCGGCC |
| iMab D600 | |
| 1 | CATA TGGCTCCGGT TGGTCTGAAA GCTCGTAACG CTGACGAATC |
| | CGGTCACGTT GTTCTGCGTT GCCGTGCTTC |
| 81 | CGGTTACACC ATCGGTCCGA TCTGCTACGA AGTTGACGTT TCCGCTGGTC |
| | AGGACGCTGG TTCCGTTCAG CGTGTTGAAA |
| 161 | TCAACATGGG TCGTACCGAA TCCGTTCTGT CCAACCTGCG TGGTCGTACC |
| | CGTTACACCT TCGCTTGCGC TGCTGACTCC |
| 241 | ACCATCTACG CTTCCTACTA CGAATGCGGT CACGGTATCT CCACCGGTGG |
| | TTACGGTTAC TCCGAATGGT CCGAACCGGT |
| 321 | TTCCCTGCTG ACCCCGTCGG CCAGCTCGGC C |
| iMab D700 | |
| 1 | CATA TGGACAAATC CACCCTGGCT GCTGTTCCGA CCTCCATCAT |
| | CGCTGACGGT CTGATGGCTT CCACCATCAC |
| 81 | CTGCGAAGCT TCCGGTTACA CCATCGGTCC GGCTTGCGTT GCTTTCGACA |
| | CCACCCTGGG TAACAACATG GGTACCTACT |
| 161 | CCGCTCCGCT GACCTCCACC ACCCTGGGTG TTGCTACCGT TACCTGCGCT |
| | GCTGACTCCA CCATCTACGC TTCCTACTAC |
| 241 | GAATGCGGTC ACGGTATCTC CACCGGTGGT TACGGTTACG CTGCTTTCTC |
| | CGTTCCGTCC GTTACCGTTA ACTTCACCGC |
| 321 | GGCCAGCTCG GCC |
| iMab D701 | |
| 1 | CATA TGATGGCTTC CACCATCACC TGCGAAGCTT CCGGTTACAC |
| | CATCGGTCCG GCTTGCGTTG CTTCGACAC |
| 81 | CACCCTGGGT AACAACATGG GTACCTACTC CGCTCCGCTG ACCTCCACCA |
| | CCCTGGGTGT TGCTACCGTT ACCTGCGCTG |
| 161 | CTGACTCCAC CATCTACGCT TCCTACTACG AATGCGGTCA CGGTATCTCC |
| | ACCGGTGGTT ACGGTTACGC TGCTTTCTCC |
| 241 | GTTCCGTCCG TTACCGTTAA CTTCACCGCG GCCAGCTCGG CC |
| iMab D702 | |
| 1 | CATA TGGCTGTTAA ATCCGTTTTC AAAGTTTCCA CCAACTTCAT |
| | CGAAAACGAC GGCACCATGG ACTCCAAACT |
| 81 | GACCTTCCGT GCTTCCGGTT ACACCATCGG TCCGCAGTGC CTGGGTTTCT |
| | TCCAGCAGGG TGTTCCGGAC GACTCCACCA |
| 161 | ACGTTGCTAC CATCAACATG GGTGGTGGTA TCACCTACTA CGGTGACTCC |
| | GTTAAATCCA TCTTCGACAT CCGTCGTGAC |
| 241 | AACGCTAAAG ACACCTACAC CGCTTCCGTT GACGACAACC AGCCGGAAGA |
| | CGTTGAAATC ACCTGCGCTG CAGACTCCAC |
| 321 | CATCTACGCT TCCTACTACG AATGCGGTCA CGGTCTGTCC ACCGGTGGTT |
| | ACGGTTACGA CCTGATCCTG CGTACCCTGC |
| 401 | AAAAAGGTAT CGACCTGTTC GTCTCGTCGG CCAGCTCGGC C |
| iMab D800 | |
| 1 | CATA TGGGTCGTTC CTCCTTCACC GTTTCCACCC CGGACATCCT |
| | GGCTGACGGT ACCATGTCCT CCACCCTGTC |
| 81 | CTGCCGTGCT TCCGGTTACA CCATCGGTCC GCAGTGCCTG TCCTTCACCC |
| | AGAACGGTGT TCCGGTTTCC ATCTCCCCGA |
| 161 | TCAACATGGG TTCCTACACC GCTACCGTTG TTGGTAACTC CGTTGGTGAC |
| | GTTACCATCA CCTGCGCTGC TGACTCCACC |
| 241 | ATCTACGCTT CCTACTACGA ATGCGGTCAC GGTATCTCCA CCGGTGGTTA |
| | CGGTTACACC CTGATCCTGT CCACCCTGCA |
| 321 | GAAAAAAATC TCCCTGTTCC CGGCCAGCTC GGCC |
| iMab D900 | |
| 1 | CATA TGCTGACCCT GACCGCTGCT GTTATCGGTG ACGGTGCTCC |
| | GGCTAACGGT AAAACCGCTA TCACCGTTGA |
| 81 | ATGCACCGCT TCCGGTTACA CCATCGGTCC GCAGTGCGTT GTTATCACCA |
| | CCAACAACGG TGCTCTGCCG AACAAAATCA |
| 161 | CCGAAAACAT GGGTGTTGCT CGTATCGCTC TGACCAACAC CACCGACGGT |
| | GTTACCGTTG TTACCTGCGC TGCTGACTCC |
| 241 | ACCATCTACG CTTCCTACTA CGAATGCGGT CACGGTATCT CCACCGGTGG |
| | TTACGGTTAC CAGCGTCAGT CCGTTGACAC |
| 321 | CCACTTCGTT AAGGCCAGCT CGGCC |
| iMab D1000 | |
| 1 | CATA TGCACAAACC GGTTATCGAA AAAGTTGACG GTGGTTACCT |
| | GTGCAAAGCT TCCGGTTACA CCATCGGTCC |
| 81 | GGAATGCATC GAACTGCTGG CTGACGGTCG TTCCTACACC AAAAACATGG |
| | GTGAAGCTTT CTTCGCTATC GACGCTTCCA |
| 161 | AAGTTACCTG CGCTGCTGAC TCCACCATCT ACGCTTCCTA CTACGAATGC |
| | GGTCACGGTA TCTCCACCGG TGGTTACGGT |
| 241 | TACCACTGGA AAGCTGAAAA CTCGGCCAGC TCGGCC |
| iMab D1001 | |
| 1 | CATA TGGTTGACGG TGGTTACCTG TGCAAAGCTT CCGGTTACAC |
| | CATCGGTCCG GAATGCATCG AACTGCTGGC |
| 81 | TGACGGTCGT TCCTACACCA AAAACATGGG TGAAGCTTTC TTCGCTATCG |
| | ACGCTTCCAA AGTTACCTGC GCTGCTGACT |
| 161 | CCACCATCTA CGCTTCCTAC TACGAATGCG GTCACGGTAT CTCCACCGGT |
| | GGTTACGGTT ACCACTGGAA AGCTGAAAAT |
| 241 | TCGGCCAGCT CGGCC |
| iMab D1100 | |
| 1 | CATA TGGCTCCGGT TGGTCTGAAA GCTCGTCTGG CTGACGAATC |
| | CGGTCACGTT GTTCTGCGTT GCCGTGCTTC |
| 81 | CGGTTACACC ATCGGTCCGA TCTGCTACGA AGTTGACGTT TCCGCTGGTA |
| | ACGACGCTGG TTCCGTTGAG CGTGTTGAAA |
| 161 | TCCTGAACAT GGGTACCGAA TCCGTTCTGT CCAACCTGCG TGGTCGTACC |
| | CGTTACACCT TCGCTTGCGC TGCTGACTCC |
| 241 | ACCATCTACG CTTCCTACTA CGAATGCGGT CACGGTATCT CCACCGGTGG |
| | TTACGGTTAC TCCGCTTGGT CCGAACCGGT |
| 321 | TTCCCTGCTG ACCCCGTCGG CCAGCTCGGC C |
| iMab D1200 | |
| 1 | CATA TGCACGGTCT GCCGATGGAA AAACGTGGTA ACTTCATCGT |
| | TGGTCAGAAC TGCTCCCTGA CCTGCCCGGC |
| 81 | TTCCGGTTAC ACCATCGGTC CGCAGTGCGT TTTCAACTGC TACTTCAACT |
| | CCGCTCTGGC ThFCTCCACC GAAAACATGG |
| 161 | GTGAATGGAC CCTGGACATG GTTTTCTCCG ACGCTGGTAT CTACACCATG |
| | TGCGCTGCTG ACTCCACCAT CTACGCTTCC |
| 241 | TACTACGAAT GCGGTCACGG TATCTCCACC GGTGGTTACG GTTACAACCC |
| | GGTTTCCCTG GGTTCCTTCG TTGTTGACTC |
| 321 | CCCGGCCAGC TCGGCC |
| iMab D1202 | |
| 1 | CATA TGATCGTTAA ACTGGTTATG GAAAAACGTG GTAACTTCGA |
| | AAACGGTCAG GACTGCAAAC TGACCATCCG |
| 81 | TGCTCCGGT TACACCATCG GTCCGGCTTG CGACGGTTTC TTCTGCCAGT |
| | TCCCGTCCGA CGACTCCTTC TCCACCGAAG |
| 161 | ACAACATGGG TGGTGGTATC ACCGTTAACG ACGCTATGAA ACCGCAGTTC |
| | GACATCCGTC GTGACAACGC TAAAGGCACC |
| 241 | TGGACCCTGT CCATGGACTT CCAGCCGGAA GGTATCTACG AAATGCAGTG |
| | CGCTGCAGAC TCCACCATCT ACGCTTCCTA |
| 321 | CTACGAATGC GGTCACGGTC TGTCCACCGG TGGTTACGGT TACGACAACC |
| | CGGTTCGTCT GGGTGGTTTC GACGTTGACG |
| 401 | TCTCGTCGGC CAGCTCGGCC |
| iMab D1300 | |
| 1 | CATA TGCTGCAGGT TGACATCAAA CCGTCCCAGG GTGAAATCTC |
| | CGTTGGTGAA TCCAAATTCT TCCTGTGCCA |
| 81 | GGCTTCCGGT TACACCATCG GTCCGTGCAT CTCCTGGTTC TCCCCGAACG |
| | GTGAAAAACT GAACATGGGT TCCTCCACCC |
| 161 | TGACCATCTA CAACGCTAAC ATCGACGACG CTGGTATCTA CAAATGCGCT |
| | GCTGACTCCA CCATCTACGC TTCCTACTAC |
| 241 | GAATGCGGTC ACGGTATCTC CACCGGTGGT TACGGTTACC AGTCCGAAGC |
| | TACCGTTAAC GTTAAAATCT TCCACGCCAG |
| 321 | CTCGGCC |
| iMab D1301 | |
| 1 | CATA TGGAATCCAA ATTCTTCCTG TGCCAGGCTT CCGGTTACAC |
| | CATCGGTCCG TGCATCTCCT GGTTCTCCCC |
| 81 | GAACGGTGAA AAACTGAACA TGGGTTCCTC CACCCTGACC ATCTACAACG |
| | CTAACATCGA CGACGCTGGT ATCTACAAAT |
| 161 | GCGCTGCTGA CTCCACCATC TACGCTTCCT ACTACGAATG CGGTCACGGT |
| | ATCTCCACCG GTGGTTACGG ITACCAGTCC |
| 241 | GAAGCTACCG TTAACGTTAA AATCTTCCAG GCCAGCTCGG CC |
| iMab D1302 | |
| 1 | CATA TGGTTGTTAA AGTTGTTATC AAACCGTCCC AGAACTTCAT |
| | CGAAAACGGT GAAGACAAAA AATTCACCTG |
| 81 | CCGTGCTTCC GGTTACACCA TCGGTCCGAA ATGCATCGGT TGGTTCTCCC |
| | AGAACCCGGA AGACGACTCC ACCAACGTTG |
| 161 | CTACCATCAA CATGGGTGGT GGTATCACCT ACTACGGTGA CTCCGTTAAA |
| | GAACGTTTCG ACATCCGTCG TGACAACGCT |
| 241 | AAAGACACCT CCACCCTGTC CATCGACGAC GCTCAGCCGG AAGACGCTGG |
| | TATCTACAAA TGCGCTGCAG ACTCCACCAT |
| 321 | CTACGCTTCC TACTACGAAT GCGGTCACGG TCTGTCCACC GGTGGTTACG |
| | GTTACGACTC CGAAGCTACC GTTGGTGTTG |
| 401 | ACATCTTCGT CTCGTCGGCC AGCTCGGCC |
| iMab D1400 | |
| 1 | CATA TGGTTCCGCG TGACCTGGAA GTTGTTGCTG CTACCCCGAC |
| | CTCCCTGCTG ATCTCCTGCG ACGCTTCCGG |
| 81 | TTACACCATC GGTCCGTACT GCATCACCTA CGGTGAAACC GGTGGTAACT |
| | CCCCGGTTCA GGAATTCACC GTTCCGAACA |
| 161 | TGGGTAAATC CACCGCTACC ATCTCCGGTC TGAAACCGGG TGTTGACTAC |
| | ACCATCACCT GCGCTGCTGA CTCCACCATC |
| 241 | TACGCTTCCT ACTACGAATG CGGTCACGGT ATCTCCACCG GTGGTTACGG |
| | TTACTCCAAA CCGATCTCCA TCAACTACCG |
| 321 | TACGGCCAGC TCGGCC |
| iMab D1500 | |
| 1 | CATA TGATCAAAGT TTACACCGAC CGTGAAAACT ACGGTGCTGT |
| | TGGTTCCCAG GTTACCCTGC ACTGCTCCGC |
| 81 | TTCCGGTTAC ACCATCGGTC CGATCTGCTT CACCTGGCGT TACCAGCCGG |
| | AAGGTGACCG TGACGCTATC TCCATCTTCC |
| 161 | ACTACAACAT GGGTGACGGT TCCATCGTTA TCCACAACCT GGACTACTCC |
| | GACAACGGTA CCTTCACCTG CGCTGCTGAC |
| 241 | TCCACCATCT ACGCTTCCTA CTACGAATGC GGTCACGGTA TCTCCACCGG |
| | TGGTTACGGT TACGTTGGTA AAACCTCCCA |
| 321 | GGTTACCCTG TACGTTTTCG AGGCCAGCTC GGCC |
| iMab D1501 | |
| 1 | CATA TGTCCCAGGT TACCCTGCAC TGCTCCGCTT CCGGTTACAC |
| | CATCGGTCCG ATCTGCTTCA CCTGGCGTTA |
| 81 | CCAGCCGGAA GGTGACCGTG ACGCTATCTC CATCTTCCAC TACAACATGG |
| | GTGACGGTTC CATCGTTATC CACAACCTGG |
| 161 | ACTACTCCGA CAACGGTACC TTCACCTGCG CTGCTGACTC CACCATCTAC |
| | GCTTCCTACT ACGAATGCGG TCACGGTATC |
| 241 | TCCACCGGTG GTTACGGTTA CGTTGGTAAA ACCTCCCAGG TTACCCTGTA |
| | CGTTTTCGAG GCCAGCTCGG CC |
| iMab D1502 | |
| 1 | CATA TGAACGTTAA AGTGGTTACC AAACGTGAAA ACTTCGGTGA |
| | AAACGGTTCC GACGTTAAAC TGACCTGCCG |
| 81 | TGCTTCCGGT TACACCATCG GTCCGATCTG CTTCGGITGG TTCTACCAGC |
| | CGGAAGGTGA CGACTCCGCT ATCTCCATCT |
| 161 | TCCACAACAT GGGTGGTGGT ATCACCGACG AAGTTGACAC CTTCAAAGAA |
| | CGTTTCGACA TCCGTCGTGA CAACGCTAAA |
| 241 | AAAACCGGCA CCATCTCCAT CGACGACCTG CAACCGTCCG ACAACGAAAC |
| | CTTCACCTGC GCTGCAGACT CCACCATCTA |
| 321 | CGCTTCCTAC TACGAATGCG GTCACGGTCT GTCCACCGGT GGTTACGGTT |
| | ACGACGGTAA AACCCGTCAG GTTGGTCTGG |
| 401 | ACGTTTTCGT CTCGTCGGCC AGCTCGGCC |
| iMab D1600 | |
| 1 | CATA TGATCAAAGT TTACACCGAC CGTGAAAACT ACGGTGCTGT |
| | TGGTTCCCAG GTTACCCTGC ACTGCTCCGC |
| 81 | TTCCGGTTAC ACCATCGGTC CGATCTGCTT CACCTGGCGT TACCAGCCGG |
| | AAGGTGACCG TGACGCTATC TCCATCTTCC |
| 161 | ACTACAACAT GGGTGACGGT TCCATCGTTA TCCACAACCT GGACTACTCC |
| | GACAACGGTA CCTTCACCTG CGCTGCTGAC |
| 241 | TCCACCATCT ACGCTTCCTA CTACGAATGC GGTCACGGTA TCTCCACCGG |
| | TGGTTACGGT TACGTTGGTA AAACCTCCCA |
| 321 | GGTTACCCTG TACGTTTTCG AGGCCAGCTC GGCC |
| iMab D1602 | |
| 1 | CATATGGCTG TTAAACCGGT TATCGGTTCC AAAGCTCCGA ACTTCGGTGA |
| | AAACGGTGAC GTTAAAACCA TCGACCGTGC |
| 81 | TTCCGGTTAC ACCATCGGTC CGACCTGCGG TGGTGTTTTC TTCCAGGGTC |
| | CGACCGACGA CTCCACCAAC GTTGCTACCA |
| 161 | TCAACATGGG TGGTGGTATC ACCTACTACG GTGACTCCGT TAAAGAAACC |
| | TTCGACATCC GTCGTGACAA CGCTAAATCC |
| 241 | ACCCGTACCG AATCCTACGA CGACAACCAG CCGGAAGGTC TGACCGAAGT |
| | TAAATGCGCT GCAGACTCCA CCATCTACGC |
| 321 | TTCCTACTAC GAATGCGGTC ACGGTCTGTC CACCGGTGGT TACGGTTACG |
| | ACGTTTCCTC CCGTCTGTAC GGTTACGACA |
| 401 | TCCTGGTCTC GTCGGCCAGC TCGGCC |
| iMab D1700 | |
| 1 | CATA TGAAAGACCC GGAAATCCAC CTGTCCGGTC CGCTGGAAGC |
| | TGGTAAACCG ATCACCGTA AATGCTCCGC |
| 81 | TTCCGGTTAC ACCATCGGTC CGCTGTGCAT CGACCTGCTG AAAGGTGACC |
| | ACCTGATGAA ATCCCAGGAA TTCAACATGG |
| 161 | GTTCCCTGGA AGTTACCTTC ACCCCGGTTA TCGAAGACAT CGGTAAAGTT |
| | CTGGTTGCG CTGCTGACTC CACCATCTAC |
| 241 | GCITCCTACT ACGAATGCGG TCACGGTATC TCCACCGGTG GTTACGGTTA |
| | CGTTCGTCAG GCTGTTAAAG AACTGCAGGT |
| 321 | TGACTCGGCC AGCTCGGCC |
| iMab D1701 | |
| 1 | CATA TGAAACCGAT CACCGTTAAA TGCTCCGCTT CCGGTTACAC |
| | CATCGGTCCG CTGTGCATCG ACCTGCTGAA |
| 81 | AGGTGACCAC CTGATGAAAT CCCAGGAATT CAACATGGGT TCCCTGGAAG |
| | TTACCTTCAC CCCGGTTATC GAAGACATCG |
| 161 | GTAAAGTTCT GGTTTGCGCT GCTGACTCCA CCATCTACGC TTCCTACTAC |
| | GAATGCGGTC ACGGTATCTC CACCGGTGGT |
| 241 | TACGGTTACG TTCGTCAGGC TGTTAAAGAA CTGCAGGTTG ACTCGGCCAG |
| | CTCGGCC |

TABLE 5

List of primers used.

| Primer | Sequence |
|---|---|
| number | 5′→3′ |
| Pr4 | CAGGAAAACAGCTATGACC |
| Pr5 | TGTAAAACGACGGCCAGT |
| Pr8 | CCTGAAACCTGAGGACACGGCC |
| Pr9 | CAGGGTCCCC/TTG/TGCCCCAG |
| Pr33 | GCTATGCCATAGCATTTTTATCC |
| Pr35 | ACAGCCAAGCTGGAGACCGT |
| Pr49 | GGTGACCTGGGTACCC/TTG/TGCCCCGG |
| Pr56 | GGAGCGC/TGAGGGGGTCTCATG |
| Pr73 | GAGGACACTGCCGTATATTAC/TTG |
| Pr75 | GAGGACACTGCAGAATATAAC/TTG |
| Pr76 | CCAGGGAAGG/CAGCGC/TGAGTT |
| Pr80 | GATGACGATCTTAAGCTCACGNNNCGTGCTGAAGGTTACACCAT |
| | TG |
| Pr81 | CGTAAATGGTAGAATCACCTGCNNNATTGTATTCTGCAGAGTCT |
| | TCC |
| Pr82 | CCGCAATGTGAAACTGGTTTGTAAAGGTGGCAATTTCGTC |
| Pr83 | CGGTAACGTCGGTACCCTGGCAACGGTAGTGGCTATCGTAG |
| Pr120 | AGGCGGGCGGCCGCAATGTGAAACTGGTTG |
| Pr121 | CACCGGCCGAGCTGGCCGACGAGACGGTAA |
| Pr129 | TATACATATGAATGTGAAACTGGTTGAAAAAG |
| Pr136 | CTTCGATATCCGTCGCGACGATGCGTCCAACACCGTTACCTTAT |
| | CG |
| Pr299 | GAGGACACGGCCACATACTACTGT |
| Pr300 | GACCAGGAGTCCTTGGCCCCAGGC |
| Pr301 | GACCAGGAGTCCTTGGCCCCA |
| Pr302 | GTTGTGGTTTTGGTGTCTTGGGTTC |
| Pr303 | CTTGGATTCTGTTGTAGGATTGGGTTG |
| Pr304 | GGGGTCTTCGCTGTGGTGC |
| Pr305 | CTTGGAGCTGGGGTCTTCGC |
| Pr306 | CCGGATCCTTAGTGGTGATGGTGATGGTGGCTTTTGCCCAGGCG |
| | GTTCATTTCTATATCGGTATAGCTGCCACCGCCACCGGCCGAGC |
| | TGGCCGACGAG |

Table 6.

Binding Characteristics of Purified iMab Variants to Lysozyme. Various purified iMabs containing either 6-,7-, or 9 β-sheets were analyzed for binding to ELK (control) and lysozyme as described in example 8, 15,19 and 23. All iMabs were purified using urea and subsequent matrix assisted refolding (Example 7), except for iMab100 which was additionally also purified by heat-induced solubilization of inclusion bodies (Example 6).

TABLE 6

| | | | | Absorbtion (450 nm) | |
|---|---|---|---|---|---|
| | No. of β- | Purification | Amount of iMab applied | ELK | Lysozyme (100 |
| iMab | sheets | procedure | per well (in 100 μl) | (control) | μg/ml) |
| 1302 | 9 | urea | ˜50 ng | 0.045 | 0.345 |
| 1602 | 9 | urea | ˜50 ng | 0.043 | 0.357 |
| 1202 | 9 | urea | ˜50 ng | 0.041 | 0.317 |
| 116 | 9 | urea | ˜50 ng | 0.042 | 0.238 |
| 101 | 7 | urea | ˜20 ng | 0.043 | 0.142 |
| 111 | 9 | urea | ˜50 ng | 0.043 | 0.420 |
| 701 | 6 | urea | ˜10 ng | 0.069 | 0.094 |
| 122 | 9 | urea | ˜50 ng | 0.051 | 0.271 |
| 1300 | 7 | urea | ˜50 ng | 0.041 | 0.325 |
| 1200 | 7 | urea | ˜5 ng | 0.040 | 0.061 |
| 900 | 7 | urea | ˜10 ng | 0.043 | 0.087 |
| 100 | 9 | urea | ˜50 ng | 0.040 | 0.494 |
| 100 | 9 | heat (60° C.) | ˜50 ng | 0.041 | 0.369 |

Table X. Binding Characteristics of purified iMab Variants to Lysozyme.

Various purified iMabs containing either 6-, 7-, or 9 β-sheets were analyzed for binding to ELK (control) and lysozyme as described in examples 8, 15,19 and 23.

All iMabs were purified using urea and subsequent matrix assisted refolding (Example 7), except for iMab100 which was additionally also purified by heat-induced solubilization of inclusion bodies (Example 6).

TABLE 7
Effect of pH shock on iMab100, measured in Elisa vs. lysozyme
before and after precipitation by potassium acetate pH 4.8.

| iMab | Signal on Elisa of | Signal on Elisa of |
|---|---|---|
| dilution | input iMab100 | pH shocked iMab100 |
| 1:10 | 0.360 | 0.390 |
| 1:100 | 0.228 | 0.263 |
| 1:1000 | 0.128 | 0.169 |
| 1:10,000 | 0.059 | 0.059 |

TABLE 8
Four examples of seven-stranded (strands-only) folds
in PDB 2.0 format to indicate spatial conformation.

| | | | | | | | | | | |
|---|---|---|---|---|---|---|---|---|---|---|
| 1NEU | | | | | | | | | | |
| ATOM | 1 | CA | GLY | 2 | −33.839 | −10.967 | −0.688 | 1.00 | 25.06 | C |
| ATOM | 2 | CA | GLY | 3 | −31.347 | −8.590 | −2.388 | 1.00 | 20.77 | C |
| ATOM | 3 | CA | GLY | 4 | −29.325 | −6.068 | −0.288 | 1.00 | 19.01 | C |
| ATOM | 4 | CA | GLY | 5 | −27.767 | −2.669 | −1.162 | 1.00 | 19.75 | C |
| ATOM | 5 | CA | GLY | 6 | −27.109 | 0.487 | 1.010 | 1.00 | 22.33 | C |
| ATOM | 6 | CA | GLY | 7 | −29.834 | 3.204 | 0.812 | 1.00 | 24.80 | C |
| ATOM | 7 | CA | GLY | 8 | −27.542 | 6.161 | 0.057 | 1.00 | 28.23 | C |
| ATOM | 8 | CA | GLY | 9 | −23.790 | 6.593 | −0.286 | 1.00 | 26.37 | C |
| ATOM | 9 | CA | GLY | 10 | −21.750 | 9.765 | −0.074 | 1.00 | 29.08 | C |
| ATOM | 10 | CA | GLY | 11 | −18.505 | 10.289 | −1.920 | 1.00 | 26.48 | C |
| ATOM | 11 | CA | GLY | 12 | −15.859 | 12.991 | −2.286 | 1.00 | 27.26 | C |
| ATOM | 12 | CA | GLY | 13 | −14.782 | 14.286 | −5.685 | 1.00 | 26.73 | C |
| ATOM | 13 | CA | GLY | 15 | −12.221 | 9.666 | −4.538 | 1.00 | 23.37 | C |
| ATOM | 14 | CA | GLY | 16 | −13.862 | 6.196 | −4.876 | 1.00 | 22.86 | C |
| ATOM | 15 | CA | GLY | 17 | −16.948 | 4.527 | −3.422 | 1.00 | 20.21 | C |
| ATOM | 16 | CA | GLY | 18 | −18.042 | 0.875 | −3.195 | 1.00 | 19.23 | C |
| ATOM | 17 | CA | GLY | 19 | −21.543 | −0.132 | −4.244 | 1.00 | 17.13 | C |
| ATOM | 18 | CA | GLY | 20 | −22.550 | −3.266 | −2.294 | 1.00 | 19.06 | C |
| ATOM | 19 | CA | GLY | 21 | −24.854 | −5.946 | −3.635 | 1.00 | 15.28 | C |
| ATOM | 20 | CA | GLY | 22 | −25.493 | −9.401 | −2.203 | 1.00 | 15.88 | C |
| ATOM | 21 | CA | GLY | 23 | −28.333 | −11.882 | −1.980 | 1.00 | 18.12 | C |
| ATOM | 22 | CA | GLY | 24 | −29.458 | −14.458 | 0.564 | 1.00 | 18.67 | C |
| ATOM | 23 | CA | GLY | 25 | −31.806 | −17.445 | 0.594 | 1.00 | 20.29 | C |
| ATOM | 24 | CA | GLY | 33 | −26.348 | −16.618 | −10.937 | 1.00 | 24.64 | C |
| ATOM | 25 | CA | GLY | 34 | −26.032 | −13.298 | −12.772 | 1.00 | 21.24 | C |
| ATOM | 26 | CA | GLY | 35 | −25.552 | −9.691 | −11.546 | 1.00 | 17.90 | C |
| ATOM | 27 | CA | GLY | 36 | −26.440 | −6.639 | −13.630 | 1.00 | 16.78 | C |
| ATOM | 28 | CA | GLY | 37 | −25.790 | −3.001 | −12.553 | 1.00 | 15.77 | C |
| ATOM | 29 | CA | GLY | 38 | −27.841 | −0.127 | −14.139 | 1.00 | 16.15 | C |
| ATOM | 30 | CA | GLY | 39 | −27.421 | 3.671 | −13.662 | 1.00 | 17.11 | C |
| ATOM | 31 | CA | GLY | 40 | −30.023 | 6.514 | −13.788 | 1.00 | 19.95 | C |
| ATOM | 32 | CA | GLY | 69 | −13.975 | 3.798 | −13.786 | 1.00 | 19.37 | C |
| ATOM | 33 | CA | GLY | 70 | −15.517 | 0.412 | −12.834 | 1.00 | 17.40 | C |
| ATOM | 34 | CA | GLY | 71 | −13.840 | −2.419 | −10.867 | 1.00 | 17.48 | C |
| ATOM | 35 | CA | GLY | 72 | −15.422 | −5.847 | −10.205 | 1.00 | 17.29 | C |
| ATOM | 36 | CA | GLY | 73 | −14.951 | −6.863 | −6.568 | 1.00 | 17.82 | C |
| ATOM | 37 | CA | GLY | 74 | −17.604 | −9.507 | −6.001 | 1.00 | 20.26 | C |
| ATOM | 38 | CA | GLY | 81 | −21.892 | −8.819 | −6.747 | 1.00 | 15.95 | C |
| ATOM | 39 | CA | GLY | 82 | −20.250 | −5.588 | −5.572 | 1.00 | 14.51 | C |
| ATOM | 40 | CA | GLY | 83 | −18.342 | −3.035 | −7.740 | 1.00 | 14.59 | C |
| ATOM | 41 | CA | GLY | 84 | −16.254 | 0.065 | −7.050 | 1.00 | 15.51 | C |
| ATOM | 42 | CA | GLY | 85 | −16.847 | 3.424 | −8.859 | 1.00 | 16.32 | C |
| ATOM | 43 | CA | GLY | 86 | −13.490 | 5.206 | −9.235 | 1.00 | 17.40 | C |
| ATOM | 44 | CA | GLY | 87 | −12.392 | 8.860 | −9.565 | 1.00 | 20.53 | C |
| ATOM | 45 | CA | GLY | 89 | −17.022 | 13.543 | −10.254 | 1.00 | 33.26 | C |
| ATOM | 46 | CA | GLY | 90 | −19.763 | 16.019 | −9.293 | 1.00 | 33.41 | C |
| ATOM | 47 | CA | GLY | 91 | −21.946 | 14.910 | −12.147 | 1.00 | 28.24 | C |
| ATOM | 48 | CA | GLY | 92 | −22.001 | 11.307 | −11.004 | 1.00 | 23.61 | C |
| ATOM | 49 | CA | GLY | 93 | −25.084 | 11.842 | −8.710 | 1.00 | 22.12 | C |
| ATOM | 50 | CA | GLY | 94 | −27.797 | 9.318 | −9.433 | 1.00 | 18.21 | C |
| ATOM | 51 | CA | GLY | 95 | −29.408 | 6.033 | −8.549 | 1.00 | 18.57 | C |
| ATOM | 52 | CA | GLY | 96 | −27.753 | 2.594 | −9.167 | 1.00 | 17.72 | C |
| ATOM | 53 | CA | GLY | 97 | −29.778 | −0.614 | −9.279 | 1.00 | 18.72 | C |
| ATOM | 54 | CA | GLY | 98 | −28.513 | −4.176 | −8.741 | 1.00 | 18.53 | C |
| ATOM | 55 | CA | GLY | 99 | −30.558 | −6.911 | −10.517 | 1.00 | 18.45 | C |
| ATOM | 56 | CA | GLY | 100 | −29.969 | −10.529 | −9.427 | 1.00 | 17.71 | C |
| ATOM | 57 | CA | GLY | 108 | −34.816 | −10.904 | −11.290 | 1.00 | 32.51 | C |
| ATOM | 58 | CA | GLY | 109 | −34.993 | −9.224 | −7.900 | 1.00 | 28.62 | C |
| ATOM | 59 | CA | GLY | 110 | −33.628 | −5.668 | −7.622 | 1.00 | 23.31 | C |
| ATOM | 60 | CA | GLY | 111 | −32.406 | −3.176 | −5.020 | 1.00 | 19.82 | C |
| ATOM | 61 | CA | GLY | 112 | −31.140 | 0.403 | −5.467 | 1.00 | 19.75 | C |
| ATOM | 62 | CA | GLY | 113 | −28.697 | 2.839 | −3.811 | 1.00 | 18.42 | C |
| ATOM | 63 | CA | GLY | 114 | −28.461 | 6.627 | −4.417 | 1.00 | 18.70 | C |
| ATOM | 64 | CA | GLY | 115 | −25.110 | 8.413 | −4.799 | 1.00 | 19.95 | C |
| ATOM | 65 | CA | GLY | 116 | −24.286 | 12.022 | −3.753 | 1.00 | 25.78 | C |
| ATOM | 66 | CA | GLY | 117 | −20.816 | 13.493 | −4.292 | 1.00 | 31.06 | C |
| ATOM | 67 | CA | GLY | 118 | −19.616 | 16.565 | −2.380 | 1.00 | 38.60 | C |
| ATOM | 68 | CA | GLY | 119 | −16.267 | 18.356 | −1.757 | 1.00 | 42.50 | C |
| TER | | | | | | | | 1.00 | | |
| 1MEL | | | | | | | | 1.00 | | |
| ATOM | 69 | CA | GLY A | 3 | −34.517 | −10.371 | −1.234 | 1.00 | 36.96 | C |
| ATOM | 70 | CA | GLY A | 4 | −31.790 | −8.022 | −2.500 | 1.00 | 28.80 | C |
| ATOM | 71 | CA | GLY A | 5 | −30.310 | −5.603 | 0.018 | 1.00 | 31.64 | C |
| ATOM | 72 | CA | GLY A | 6 | −27.884 | −2.957 | −1.209 | 1.00 | 24.68 | C |
| ATOM | 73 | CA | GLY A | 7 | −25.500 | −1.042 | 1.073 | 1.00 | 21.60 | C |
| ATOM | 74 | CA | GLY A | 8 | −23.005 | 1.710 | 0.458 | 1.00 | 17.27 | C |
| ATOM | 75 | CA | GLY A | 9 | −23.567 | 4.843 | −1.499 | 1.00 | 18.58 | C |
| ATOM | 76 | CA | GLY A | 10 | −23.648 | 8.381 | −0.100 | 1.00 | 16.78 | C |
| ATOM | 77 | CA | GLY A | 11 | −21.736 | 11.497 | −1.127 | 1.00 | 11.36 | C |
| ATOM | 78 | CA | GLY A | 12 | −18.140 | 11.942 | −1.980 | 1.00 | 8.84 | C |
| ATOM | 79 | CA | GLY A | 13 | −16.006 | 14.459 | −3.746 | 1.00 | 11.16 | C |
| ATOM | 80 | CA | GLY A | 14 | −15.243 | 14.091 | −7.418 | 1.00 | 10.62 | C |
| ATOM | 81 | CA | GLY A | 16 | −12.920 | 9.782 | −4.554 | 1.00 | 7.90 | C |
| ATOM | 82 | CA | GLY A | 17 | −14.217 | 6.244 | −4.387 | 1.00 | 7.54 | C |
| ATOM | 83 | CA | GLY A | 18 | −17.233 | 4.430 | −2.940 | 1.00 | 6.47 | C |
| ATOM | 84 | CA | GLY A | 19 | −18.104 | 0.790 | −2.418 | 1.00 | 5.43 | C |
| ATOM | 85 | CA | GLY A | 20 | −21.615 | −0.610 | −2.878 | 1.00 | 8.09 | C |
| ATOM | 86 | CA | GLY A | 21 | −22.724 | −4.116 | −1.837 | 1.00 | 8.76 | C |
| ATOM | 87 | CA | GLY A | 22 | −25.644 | −6.399 | −2.433 | 1.00 | 8.33 | C |
| ATOM | 88 | CA | GLY A | 23 | −26.642 | −9.585 | −0.745 | 1.00 | 13.77 | C |
| ATOM | 89 | CA | GLY A | 24 | −29.318 | −11.732 | −2.396 | 1.00 | 17.53 | C |
| ATOM | 90 | CA | GLY A | 25 | −31.340 | −13.525 | 0.256 | 1.00 | 23.23 | C |
| ATOM | 91 | CA | GLY A | 26 | −33.969 | −16.194 | 0.081 | 1.00 | 25.25 | C |
| ATOM | 92 | CA | GLY A | 32 | −27.171 | −16.809 | −12.135 | 1.00 | 5.00 | C |
| ATOM | 93 | CA | GLY A | 33 | −26.411 | −13.068 | −12.502 | 1.00 | 4.64 | C |
| ATOM | 94 | CA | GLY A | 34 | −26.109 | −10.036 | −10.166 | 1.00 | 2.48 | C |
| ATOM | 95 | CA | GLY A | 35 | −25.386 | −6.603 | −11.615 | 1.00 | 2.00 | C |
| ATOM | 96 | CA | GLY A | 36 | −25.845 | −2.895 | −11.151 | 1.00 | 3.40 | C |
| ATOM | 97 | CA | GLY A | 37 | −28.032 | −0.410 | −12.986 | 1.00 | 6.64 | C |
| ATOM | 98 | CA | GLY A | 38 | −28.158 | 3.311 | −12.472 | 1.00 | 8.74 | C |
| ATOM | 99 | CA | GLY A | 39 | −30.731 | 6.025 | −13.179 | 1.00 | 19.24 | C |
| ATOM | 100 | CA | GLY A | 67 | −12.972 | 2.698 | −13.036 | 1.00 | 12.68 | C |
| ATOM | 101 | CA | GLY A | 68 | −15.482 | 0.261 | −11.584 | 1.00 | 8.15 | C |
| ATOM | 102 | CA | GLY A | 69 | −14.843 | −3.306 | −10.511 | 1.00 | 8.39 | C |
| ATOM | 103 | CA | GLY A | 70 | −17.520 | −5.831 | −9.556 | 1.00 | 5.47 | C |
| ATOM | 104 | CA | GLY A | 71 | −16.742 | −8.900 | −7.482 | 1.00 | 8.48 | C |
| ATOM | 105 | CA | GLY A | 72 | −18.459 | −11.484 | −5.287 | 1.00 | 18.00 | C |
| ATOM | 106 | CA | GLY A | 79 | −21.342 | −8.788 | −4.615 | 1.00 | 9.55 | C |
| ATOM | 107 | CA | GLY A | 80 | −19.553 | −5.399 | −4.519 | 1.00 | 4.94 | C |
| ATOM | 108 | CA | GLY A | 81 | −18.901 | −2.601 | −6.858 | 1.00 | 4.92 | C |
| ATOM | 109 | CA | GLY A | 82 | −15.755 | −0.638 | −6.085 | 1.00 | 7.44 | C |
| ATOM | 110 | CA | GLY A | 83 | −16.081 | 2.748 | −7.765 | 1.00 | 6.55 | C |
| ATOM | 111 | CA | GLY A | 84 | −12.869 | 4.759 | −8.177 | 1.00 | 9.49 | C |
| ATOM | 112 | CA | GLY A | 85 | −12.245 | 8.019 | −10.028 | 1.00 | 12.54 | C |
| ATOM | 113 | CA | GLY A | 87 | −16.853 | 12.035 | −11.452 | 1.00 | 15.46 | C |
| ATOM | 114 | CA | GLY A | 88 | −20.054 | 14.055 | −10.955 | 1.00 | 13.94 | C |
| ATOM | 115 | CA | GLY A | 89 | −21.497 | 12.384 | −14.095 | 1.00 | 18.68 | C |
| ATOM | 116 | CA | GLY A | 90 | −21.637 | 9.217 | −11.955 | 1.00 | 9.55 | C |
| ATOM | 117 | CA | GLY A | 91 | −24.288 | 10.888 | −9.734 | 1.00 | 6.54 | C |
| ATOM | 118 | CA | GLY A | 92 | −27.349 | 8.637 | −9.936 | 1.00 | 6.38 | C |
| ATOM | 119 | CA | GLY A | 93 | −29.573 | 6.105 | −8.204 | 1.00 | 6.33 | C |
| ATOM | 120 | CA | GLY A | 94 | −27.866 | 2.727 | −8.154 | 1.00 | 6.08 | C |
| ATOM | 121 | CA | GLY A | 95 | −29.928 | −0.377 | −8.312 | 1.00 | 10.12 | C |
| ATOM | 122 | CA | GLY A | 96 | −28.884 | −3.923 | −7.638 | 1.00 | 10.76 | C |
| ATOM | 123 | CA | GLY A | 97 | −30.439 | −6.641 | −9.794 | 1.00 | 9.08 | C |
| ATOM | 124 | CA | GLY A | 98 | −30.321 | −10.442 | −10.097 | 1.00 | 8.28 | C |
| ATOM | 125 | CA | GLY A | 122 | −35.231 | −9.331 | −9.016 | 1.00 | 15.49 | C |
| ATOM | 126 | CA | GLY A | 123 | −34.520 | −5.802 | −8.079 | 1.00 | 12.37 | C |
| ATOM | 127 | CA | GLY A | 124 | −33.455 | −4.050 | −4.953 | 1.00 | 14.34 | C |
| ATOM | 128 | CA | GLY A | 125 | −33.918 | −0.696 | −3.317 | 1.00 | 22.49 | C |
| ATOM | 129 | CA | GLY A | 126 | −32.054 | 2.128 | −5.022 | 1.00 | 17.72 | C |
| ATOM | 130 | CA | GLY A | 127 | −29.137 | 3.817 | −3.342 | 1.00 | 16.51 | C |
| ATOM | 131 | CA | GLY A | 128 | −28.275 | 7.401 | −4.265 | 1.00 | 16.84 | C |
| ATOM | 132 | CA | GLY A | 129 | −24.678 | 8.216 | −4.904 | 1.00 | 11.90 | C |
| ATOM | 133 | CA | GLY A | 130 | −23.878 | 11.873 | −5.299 | 1.00 | 8.75 | C |
| ATOM | 134 | CA | GLY A | 131 | −20.508 | 13.061 | −6.466 | 1.00 | 14.37 | C |
| ATOM | 135 | CA | GLY A | 132 | −19.632 | 16.597 | −5.198 | 1.00 | 23.32 | C |
| ATOM | 136 | CA | GLY A | 133 | −17.732 | 19.533 | −6.720 | 1.00 | 36.14 | C |
| TER | | | | | | | | | | |
| 1F97 | | | | | | | | | | |
| ATOM | 137 | CA | GLY A | 29 | −33.679 | −11.517 | −0.808 | 1.00 | 41.25 | C |
| ATOM | 138 | CA | GLY A | 30 | −31.468 | −8.740 | −2.170 | 1.00 | 22.43 | C |
| ATOM | 139 | CA | GLY A | 31 | −30.250 | −5.886 | 0.038 | 1.00 | 24.73 | C |
| ATOM | 140 | CA | GLY A | 32 | −27.706 | −3.105 | 0.530 | 1.00 | 20.95 | C |
| ATOM | 141 | CA | GLY A | 33 | −25.811 | −1.723 | 3.523 | 1.00 | 28.77 | C |
| ATOM | 142 | CA | GLY A | 34 | −26.349 | 1.878 | 2.419 | 1.00 | 33.48 | C |
| ATOM | 143 | CA | GLY A | 35 | −29.027 | 3.067 | −0.012 | 1.00 | 27.47 | C |
| ATOM | 144 | CA | GLY A | 36 | −27.980 | 6.732 | 0.249 | 1.00 | 29.20 | C |
| ATOM | 145 | CA | GLY A | 37 | −24.279 | 6.955 | −0.586 | 1.00 | 23.99 | C |
| ATOM | 146 | CA | GLY A | 38 | −22.275 | 10.179 | −0.393 | 1.00 | 24.19 | C |
| ATOM | 147 | CA | GLY A | 39 | −18.592 | 10.286 | −1.302 | 1.00 | 15.35 | C |
| ATOM | 148 | CA | GLY A | 40 | −16.117 | 13.059 | −2.218 | 1.00 | 12.64 | C |
| ATOM | 149 | CA | GLY A | 41 | −15.286 | 13.515 | −5.906 | 1.00 | 9.24 | C |
| ATOM | 150 | CA | GLY A | 43 | −12.412 | 8.992 | −4.540 | 1.00 | 14.44 | C |
| ATOM | 151 | CA | GLY A | 44 | −13.115 | 5.267 | −4.685 | 1.00 | 21.52 | C |
| ATOM | 152 | CA | GLY A | 45 | −16.460 | 3.862 | −3.630 | 1.00 | 22.48 | C |
| ATOM | 153 | CA | GLY A | 46 | −18.082 | 0.444 | −3.532 | 1.00 | 21.22 | C |
| ATOM | 154 | CA | GLY A | 47 | −21.817 | 0.219 | −4.135 | 1.00 | 20.06 | C |
| ATOM | 155 | CA | GLY A | 48 | −22.797 | −2.825 | −2.072 | 1.00 | 13.56 | C |
| ATOM | 156 | CA | GLY A | 49 | −25.390 | −5.432 | −3.034 | 1.00 | 19.14 | C |
| ATOM | 157 | CA | GLY A | 50 | −25.724 | −8.537 | −0.876 | 1.00 | 22.49 | C |
| ATOM | 158 | CA | GLY A | 51 | −28.046 | −11.470 | −1.549 | 1.00 | 20.58 | C |
| ATOM | 159 | CA | GLY A | 52 | −28.951 | −14.871 | −0.105 | 1.00 | 24.55 | C |
| ATOM | 160 | CA | GLY A | 53 | −30.893 | −17.875 | −1.353 | 1.00 | 17.82 | C |
| ATOM | 161 | CA | GLY A | 57 | −26.875 | −15.797 | −9.701 | 1.00 | 10.39 | C |
| ATOM | 162 | CA | GLY A | 58 | −26.674 | −13.408 | −12.632 | 1.00 | 8.00 | C |
| ATOM | 163 | CA | GLY A | 59 | −25.845 | −9.939 | −11.318 | 1.00 | 7.62 | C |
| ATOM | 164 | CA | GLY A | 60 | −26.653 | −6.841 | −13.371 | 1.00 | 6.84 | C |
| ATOM | 165 | CA | GLY A | 61 | −26.457 | −3.109 | −12.711 | 1.00 | 8.48 | C |
| ATOM | 166 | CA | GLY A | 62 | −28.184 | −0.168 | −14.336 | 1.00 | 13.92 | C |
| ATOM | 167 | CA | GLY A | 63 | −27.611 | 3.567 | −14.043 | 1.00 | 11.89 | C |
| ATOM | 168 | CA | GLY A | 64 | −30.532 | 5.981 | −14.200 | 1.00 | 23.18 | C |
| ATOM | 169 | CA | GLY A | 85 | −12.888 | 2.268 | −13.813 | 1.00 | 17.93 | C |
| ATOM | 170 | CA | GLY A | 86 | −15.508 | −0.149 | −12.447 | 1.00 | 15.44 | C |
| ATOM | 171 | CA | GLY A | 87 | −14.603 | −3.542 | −11.016 | 1.00 | 16.79 | C |
| ATOM | 172 | CA | GLY A | 88 | −17.118 | −6.318 | −10.381 | 1.00 | 14.07 | C |
| ATOM | 173 | CA | GLY A | 89 | −17.389 | −8.592 | −7.349 | 1.00 | 16.75 | C |
| ATOM | 174 | CA | GLY A | 91 | −20.798 | −8.262 | −3.202 | 1.00 | 17.13 | C |
| ATOM | 175 | CA | GLY A | 92 | −20.995 | −5.059 | −5.247 | 1.00 | 12.84 | C |
| ATOM | 176 | CA | GLY A | 93 | −19.287 | −2.826 | −7.795 | 1.00 | 12.33 | C |
| ATOM | 177 | CA | GLY A | 94 | −16.243 | −0.709 | −6.986 | 1.00 | 12.43 | C |
| ATOM | 178 | CA | GLY A | 95 | −15.485 | 2.596 | −8.696 | 1.00 | 11.12 | C |
| ATOM | 179 | CA | GLY A | 96 | −11.765 | 3.339 | −8.675 | 1.00 | 16.95 | C |
| ATOM | 180 | CA | GLY A | 97 | −12.855 | 7.004 | −8.899 | 1.00 | 21.11 | C |
| ATOM | 181 | CA | GLY A | 99 | −16.935 | 12.349 | −10.689 | 1.00 | 22.37 | C |
| ATOM | 182 | CA | GLY A | 100 | −19.974 | 14.613 | −10.361 | 1.00 | 23.75 | C |
| ATOM | 183 | CA | GLY A | 101 | −21.190 | 13.009 | −13.619 | 1.00 | 22.84 | C |
| ATOM | 184 | CA | GLY A | 102 | −21.820 | 9.789 | −11.695 | 1.00 | 15.51 | C |
| ATOM | 185 | CA | GLY A | 103 | −24.651 | 11.224 | −9.565 | 1.00 | 11.57 | C |
| ATOM | 186 | CA | GLY A | 104 | −27.817 | 9.170 | −9.882 | 1.00 | 9.52 | C |
| ATOM | 187 | CA | GLY A | 105 | −29.401 | 5.897 | −8.871 | 1.00 | 12.64 | C |
| ATOM | 188 | CA | GLY A | 106 | −27.851 | 2.484 | −9.413 | 1.00 | 7.32 | C |
| ATOM | 189 | CA | GLY A | 107 | −30.057 | −0.590 | −9.334 | 1.00 | 7.52 | C |
| ATOM | 190 | CA | GLY A | 108 | −28.588 | −3.984 | −8.524 | 1.00 | 9.97 | C |
| ATOM | 191 | CA | GLY A | 109 | −30.576 | −6.743 | −10.211 | 1.00 | 12.91 | C |
| ATOM | 192 | CA | GLY A | 110 | −29.973 | −10.300 | −9.043 | 1.00 | 11.27 | C |
| ATOM | 193 | CA | GLY A | 118 | −35.528 | −12.495 | −8.616 | 1.00 | 13.29 | C |
| ATOM | 194 | CA | GLY A | 119 | −34.748 | −9.395 | −6.594 | 1.00 | 15.55 | C |
| ATOM | 195 | CA | GLY A | 120 | −33.436 | −5.837 | −6.855 | 1.00 | 11.36 | C |
| ATOM | 196 | CA | GLY A | 121 | −32.267 | −2.894 | −4.778 | 1.00 | 11.36 | C |
| ATOM | 197 | CA | GLY A | 122 | −31.635 | 0.758 | −5.660 | 1.00 | 11.14 | C |
| ATOM | 198 | CA | GLY A | 123 | −28.791 | 2.935 | −4.379 | 1.00 | 11.58 | C |
| ATOM | 199 | CA | GLY A | 124 | −28.626 | 6.699 | −4.773 | 1.00 | 15.52 | C |
| ATOM | 200 | CA | GLY A | 125 | −25.128 | 8.065 | −5.281 | 1.00 | 9.61 | C |
| ATOM | 201 | CA | GLY A | 126 | −24.275 | 11.677 | −4.483 | 1.00 | 10.84 | C |
| ATOM | 202 | CA | GLY A | 127 | −20.739 | 12.778 | −5.330 | 1.00 | 10.58 | C |
| ATOM | 203 | CA | GLY A | 128 | −19.667 | 15.540 | −2.929 | 1.00 | 14.41 | C |
| ATOM | 204 | CA | GLY A | 129 | −18.355 | 18.818 | −4.335 | 1.00 | 12.32 | C |
| TER | | | | | | | | | | |
| 1DQT | | | | | | | | | | |
| ATOM | 205 | CA | GLY C | 2 | −37.000 | −7.803 | −3.232 | 1.00 | 35.96 | C |
| ATOM | 206 | CA | GLY C | 3 | −33.582 | −6.481 | −4.122 | 1.00 | 30.20 | C |
| ATOM | 207 | CA | GLY C | 4 | −31.887 | −3.962 | −1.908 | 1.00 | 27.24 | C |
| ATOM | 208 | CA | GLY C | 5 | −28.640 | −2.044 | −1.950 | 1.00 | 23.16 | C |
| ATOM | 209 | CA | GLY C | 6 | −27.069 | 0.720 | 0.216 | 1.00 | 22.73 | C |
| ATOM | 210 | CA | GLY C | 7 | −28.256 | 4.289 | −0.273 | 1.00 | 24.33 | C |
| ATOM | 211 | CA | GLY C | 8 | −24.800 | 5.874 | −0.329 | 1.00 | 21.64 | C |
| ATOM | 212 | CA | GLY C | 9 | −21.252 | 4.822 | −1.130 | 1.00 | 20.25 | C |
| ATOM | 213 | CA | GLY C | 10 | −18.186 | 7.087 | −0.970 | 1.00 | 20.37 | C |
| ATOM | 214 | CA | GLY C | 11 | −15.855 | 5.961 | −3.761 | 1.00 | 22.46 | C |
| ATOM | 215 | CA | GLY C | 12 | −12.159 | 5.548 | −2.990 | 1.00 | 24.30 | C |
| ATOM | 216 | CA | GLY C | 14 | −8.661 | 5.520 | −6.931 | 1.00 | 30.82 | C |
| ATOM | 217 | CA | GLY C | 15 | −12.218 | 5.495 | −8.241 | 1.00 | 25.29 | C |
| ATOM | 218 | CA | GLY C | 16 | −13.342 | 2.240 | −6.604 | 1.00 | 21.63 | C |
| ATOM | 219 | CA | GLY C | 17 | −16.853 | 1.719 | −5.287 | 1.00 | 20.22 | C |
| ATOM | 220 | CA | GLY C | 18 | −17.869 | −1.533 | −3.647 | 1.00 | 20.86 | C |
| ATOM | 221 | CA | GLY C | 19 | −21.187 | −2.475 | −2.147 | 1.00 | 21.25 | C |
| ATOM | 222 | CA | GLY C | 20 | −23.544 | −5.395 | −1.581 | 1.00 | 21.73 | C |
| ATOM | 223 | CA | GLY C | 21 | −26.665 | −6.116 | −3.611 | 1.00 | 22.97 | C |
| ATOM | 224 | CA | GLY C | 22 | −29.032 | −8.318 | −1.684 | 1.00 | 26.59 | C |
| ATOM | 225 | CA | GLY C | 23 | −32.087 | −10.258 | −2.722 | 1.00 | 26.47 | C |
| ATOM | 226 | CA | GLY C | 24 | −34.892 | −12.390 | −1.373 | 1.00 | 31.54 | C |
| ATOM | 227 | CA | GLY C | 25 | −36.216 | −14.994 | −1.386 | 1.00 | 31.33 | C |
| ATOM | 228 | CA | GLY C | 32 | −28.221 | −15.396 | −10.763 | 1.00 | 21.25 | C |
| ATOM | 229 | CA | GLY C | 33 | −27.582 | −12.861 | −13.499 | 1.00 | 21.86 | C |
| ATOM | 230 | CA | GLY C | 34 | −26.676 | −9.507 | −11.974 | 1.00 | 19.78 | C |
| ATOM | 231 | CA | GLY C | 35 | −26.814 | −6.238 | −13.884 | 1.00 | 19.11 | C |
| ATOM | 232 | CA | GLY C | 36 | −25.505 | −2.810 | −12.890 | 1.00 | 19.23 | C |
| ATOM | 233 | CA | GLY C | 37 | −27.371 | 0.131 | −14.384 | 1.00 | 25.95 | C |
| ATOM | 234 | CA | GLY C | 38 | −26.592 | 3.830 | −14.106 | 1.00 | 30.90 | C |
| ATOM | 235 | CA | GLY C | 39 | −29.761 | 5.879 | −13.906 | 1.00 | 40.84 | C |
| ATOM | 236 | CA | GLY C | 66 | −16.463 | −4.323 | −12.728 | 1.00 | 23.17 | C |
| ATOM | 237 | CA | GLY C | 67 | −15.869 | −7.277 | −10.506 | 1.00 | 22.56 | C |
| ATOM | 238 | CA | GLY C | 68 | −17.716 | −9.180 | −7.811 | 1.00 | 21.60 | C |
| ATOM | 239 | CA | GLY C | 69 | −18.545 | −12.367 | −5.959 | 1.00 | 22.69 | C |
| ATOM | 240 | CA | GLY C | 75 | −23.757 | −10.273 | −4.597 | 1.00 | 23.03 | C |
| ATOM | 241 | CA | GLY C | 76 | −20.713 | −8.179 | −3.648 | 1.00 | 24.30 | C |
| ATOM | 242 | CA | GLY C | 77 | −20.192 | −5.631 | −6.425 | 1.00 | 23.46 | C |
| ATOM | 243 | CA | GLY C | 78 | −17.130 | −3.554 | −7.209 | 1.00 | 25.74 | C |
| ATOM | 244 | CA | GLY C | 79 | −17.159 | −0.822 | −9.864 | 1.00 | 26.08 | C |
| ATOM | 245 | CA | GLY C | 80 | −13.722 | 0.567 | −10.770 | 1.00 | 29.15 | C |
| ATOM | 246 | CA | GLY C | 81 | −12.154 | 3.299 | −12.879 | 1.00 | 26.23 | C |
| ATOM | 247 | CA | GLY C | 84 | −15.857 | 12.510 | −10.546 | 1.00 | 24.79 | C |
| ATOM | 248 | CA | GLY C | 85 | −18.200 | 12.785 | −13.517 | 1.00 | 25.37 | C |
| ATOM | 249 | CA | GLY C | 86 | −19.382 | 9.218 | −12.817 | 1.00 | 25.21 | C |
| ATOM | 250 | CA | GLY C | 87 | −20.993 | 10.374 | −9.582 | 1.00 | 23.95 | C |
| ATOM | 251 | CA | GLY C | 88 | −24.588 | 9.210 | −9.761 | 1.00 | 23.67 | C |
| ATOM | 252 | CA | GLY C | 89 | −27.227 | 6.570 | −9.098 | 1.00 | 22.75 | C |
| ATOM | 253 | CA | GLY C | 90 | −26.436 | 2.913 | −9.751 | 1.00 | 23.39 | C |
| ATOM | 254 | CA | GLY C | 91 | −29.150 | 0.276 | −9.841 | 1.00 | 22.55 | C |
| ATOM | 255 | CA | GLY C | 92 | −28.491 | −3.332 | −9.011 | 1.00 | 22.29 | C |
| ATOM | 256 | CA | GLY C | 93 | −30.706 | −5.811 | −10.865 | 1.00 | 20.45 | C |
| ATOM | 257 | CA | GLY C | 94 | −30.958 | −9.487 | −9.970 | 1.00 | 20.73 | C |
| ATOM | 258 | CA | GLY C | 105 | −35.975 | −7.679 | −9.086 | 1.00 | 24.94 | C |
| ATOM | 259 | CA | GLY C | 106 | −34.133 | −4.376 | −8.832 | 1.00 | 25.24 | C |
| ATOM | 260 | CA | GLY C | 107 | −32.992 | −2.157 | −5.970 | 1.00 | 24.05 | C |
| ATOM | 261 | CA | GLY C | 108 | −33.717 | 1.590 | −5.665 | 1.00 | 25.85 | C |
| ATOM | 262 | CA | GLY C | 109 | −30.127 | 2.411 | −6.479 | 1.00 | 23.77 | C |
| ATOM | 263 | CA | GLY C | 110 | −26.942 | 3.329 | −4.666 | 1.00 | 22.46 | C |
| ATOM | 264 | CA | GLY C | 111 | −25.759 | 6.950 | −4.824 | 1.00 | 24.13 | C |
| ATOM | 265 | CA | GLY C | 112 | −22.065 | 6.752 | −5.605 | 1.00 | 22.83 | C |
| ATOM | 266 | CA | GLY C | 113 | −20.136 | 9.957 | −4.898 | 1.00 | 23.40 | C |
| ATOM | 267 | CA | GLY C | 114 | −16.799 | 10.239 | −6.594 | 1.00 | 25.42 | C |
| END | | | | | | | | | | |

Table 9

PROSAII results (zp-comp) and values for the objective function from MODELLER for 7-stranded iMab proteins. Lower values correspond to iMab proteins which are more likely to fold correctly.

TABLE 9

| | zp-comb | objective function |
|---|---|---|
| molecule | (lower is better) | (lower is better) |
| iMab 101 | −5.63 | 853 |
| iMab 201 | −5.34 | 683 |
| iMab IS003 | −4.29 | 860 |
| iMab IS004 | −1.49 | 2744 |
| iMab 300 | −6.28 | 854 |
| iMab IS006 | −3.29 | 912 |
| iMab IS007 | −2.71 | 1558 |
| iMab IS008 | −1.10 | 808 |
| iMab IS009 | −3.70 | 1623 |
| iMab IS0010 | −2.85 | 2704 |
| iMab 400 | −5.58 | 734 |
| iMab IS0012 | −5.35 | 889 |
| iMab IS0013 | −2.85 | 1162 |
| iMab IS0014 | −2.92 | 924 |
| iMab IS0015 | −3.48 | 925 |
| iMab IS0016 | −3.23 | 837 |
| iMab 500 | −3.94 | 1356 |
| iMab IS0018 | −2.97 | 867 |
| iMab IS0019 | −3.11 | 1366 |
| iMab 600 | −4.15 | 880 |
| iMab 700 | −3.94 | 1111 |
| iMab 800 | −3.68 | 653 |
| iMab 900 | −4.65 | 833 |
| iMab 1000 | −3.57 | 631 |
| iMab IS0025 | −2.79 | 1080 |
| iMab 1100 | −4.07 | 823 |
| iMab IS0027 | −3.59 | 809 |
| iMab IS0028 | −3.51 | 1431 |
| iMab 1200 | −2.66 | 783 |
| iMab 1300 | −3.18 | 1463 |
| iMab IS0031 | −2.98 | 1263 |
| iMab IS0032 | −3.84 | 896 |
| iMab 1400 | −5.17 | 939 |
| iMab IS0034 | −4.38 | 966 |
| iMab IS0035 | −3.86 | 966 |
| iMab IS0036 | −3.29 | 862 |
| iMab IS0037 | −3.45 | 874 |
| iMab IS0038 | −2.80 | 792 |
| iMab IS0039 | −4.44 | 1858 |
| iMab IS0040 | −5.01 | 751 |
| iMab IS0041 | −2.70 | 907 |
| iMab IS0042 | −3.14 | 837 |
| iMab IS0043 | −2.80 | 1425 |
| iMab IS0044 | −3.27 | 1492 |
| iMab IS0045 | −3.56 | 1794 |
| iMab IS0046 | −3.79 | 832 |

TABLE 10

Example amino acid sequences less likely to fold as seven
stranded iMab proteins

IMABIS003
IVLTQS-P--ASLAV-S-----LGQRATISCRASGYTIGPS-FMNWFQQKP------G--
Q--PP-K--LLIYANMGDFSLNI-H--P--M-EE-EDTA-MYFCAADSTIYASYYE
CGHGISTGGYGYLTTGAGTKVELKR
IMABIS004
PTVSIF-P--P-SSE-QL----TSGGASVVCFASGYTIGPI-NVKWKIDGS------E--
--------------NMGSSTLTL-T--K--D-E---YERHN--SYTCAADSTIYASYYE
CGHGISTGGYGYPIVKSFNRNE---
IMABIS006
TPPSVY-P--L-APG-SAAQTNSMVTLGCLVKASGYTIGPE-PVTVTWNSG------S--
L--SS-G-VHTFPNMGTLSSSV-T--V--P-SSTWPSETV---TCNCAADSTIYASYYE
CGHGISTGGYGY-STKVDKKIVPK-
IMABIS007
IASPAKTH--E-KTP-I-----EGRPFQLDCVASGYTIGP--LITWKKRLSGADPN----
--------------NMG-GNLYF-T--I--V-TK---EDVSDIYKYVCAADSTIYASYYE
CGHGISTGGYGYEVVLVEYEIKGVT
IMABIS008
PVLKDQPA--E-VLF-R-----ENNPTVLECIASGYTIGPV-KYSWKKDGKSYNW-----
Q--EH-N--AALRKNMGEGSLVF-L--R--P-QA---SDEG---HYQCAADSTIYASYYE
CGHGISTGGYGYVASSRVISFRKTT
IMABIS009
KYEQKPEK--V-IVV-K-----QGQDVTIPCKASGYTIGPP-NVVWSHNAKP--------
--------------NMGDSGLVI-K--G--V-KN---GDKG-YYGCAADSTIYASYYE
CGHGISTGGYGY-DKYFETLVQVN-
IMABIS010
VPQYVS-K--D-MMA-K-----AGDVTMIYCMASGYTIGPG-YPNYFKNGKDVN------
--------------NMGGKRLLF-K--T--T-LP---EDEG---VYTCAADSTIYASYYE
CGHGISTGGYGY-PQKHSLKLTVVS
IMABIS012
IQMTQS-P-SS-LSA-S-----VGDRVTITCSASGYTIGPN-YLNWYQQKP------G--
K--AP-K--VLIYFNMGDFTLTI-S--S--L-QP---EDFA---TYYCAADSTIYASYYE
CGHGISTGGYGYWTFGQGTKVEIKR
IMABIS013
PSVFIF-P--P-SDE-Q----LKSGTASVVCLASGYTIGPA-KVQWKVD-----------
----N-A--LQS--NMGSSTLTL-S--K--A-DY---EKHK---VYACAADSTIYASYYE
CGHGISTGGYGYPVTKSFNRGEC--
IMABIS014
KGPSVF-P--L-APS-SKSTSGGTAALGCLVKASGYTIGPE-PVTVSWMSG------A--
L--TS-G--VHTFPNMGSLSSVV-T--V--P-SSSLGTQTY---ICNCAADSTIYASYYE
CGHGISTGGYGY-NTKVDKKVEPKS
IMABIS015
NPPHNL-S--V-INSEE-----LSSILKITWTASGYTIGPL-KYNIQYRTKD-----A--
S--TW-S--QIPP-NMGRSSFTV-Q--D--L-KP---FTEY---VFRCAADSTIYASYYE
CGHGISTGGYGYSDWSEEASGITYE
IMABIS016
EKPKNL-S--C-IV--N-----EGKKMRCEWDASGYTIGPT-NFTLKSEWA------T--
H--K--F--ADCKANMGPTSCTVDY--S--T-VY---FNVI---EVWCAADSTIYASYYE
CGHGISTGGYGYKVTSDHINFDPVY
IMABIS018
NAPKLT-G-IT-CQA-D--------KAEIHWEASGYTIGPL-HYTIQFNTS------F--
TPASW-D--AAYEKNMGDSSFVV-Q--M--S--P---WANY---TFRCAADSTIYASYYE
CGHGISTGGYGYSPPSAHSDSCT--
IMABIS019
GPEELL-C--F-TE--------RLEDLVCFWEASGYTIGPG-QYSFSYQLE------D--
E--PW-K--LCR--NMGRFWCSL-P--TADT-SS---FVPL---ELRCAARSTIYASYYE
CGHGISTGGYGYGAPRYHRVIHINE
IMABIS020
APVGLV-A--R-LA--D-----ESGHVVLRWLASGYTIGPI-RYEVDVSAG------Q--
GAG-S-V--QRVEINMGRTECVL-S--N--L-RG---RTRY---TFACAADSTIYASYYE
CGHGISTGGYGYSEWSEPVSLLTPS
IMABIS025
GPEELL-C--F-TE--------RLEDLVCFWEASGYTIGPPGNYSFSYQLE------D--
E--PW-K--LCR--NMGRFWCSL-P--TADT-SS---FVPL---ELRCAADSTIYASYYE
CGHGISTGGYGYGAPRYHRVIHINE
IMABIS027
APVGLV-A--R-LAD-E------SGHVVLRWLASGYTIGPI-RYEVDVSAG------QGA
G--SV-Q--RVEILNMG-TECVL-S--N--L-RG---RTRY---TFACAADSTIYASYYE
CGHGISTGGYGYSEWSEPVSLLTPS
IMABIS028
GPEELL-C--F-TE--------RLEDLVCFWEASGYTIGPG-QYSFSYQLE------D--
E--PW-K--LCR--NMGRFWCSL-PTAD--T-SS---FVPL---ELRCAADSTIYASYYE
CGHGISTGGYGYGAPRYHRVIHINE
IMABIS031
LMFKNAPT-PQ-EFK--------EGEDAVIVCDASGYTIGPP-TIIWKHKGRDV------
--------------NMGNNYLQI-R--G--I-KK---TDEG---TYRCAADSTIYASYYE
CGHGISTGGYGYINFK-DIQVIV--
IMABIS032
DSPTGI-D--F-SD--I-----TANSFTVHWIASGYTIGPT-GYRIRHHPE------H--
F--SGRP--REDRVNMGRNSITL-T--N--L-TP---GTEY---VVSCAADSTIYASYYE
CGHGISTGGYGYSPL-LIGQQSTVS
IMABIS034
SPPTNL-H--L-EAN-P-----DTGVLTVSWEASGYTIGPT-GYRITTTPT------N--
G--QQGN-SLEEVVNMGQSSCTF-D--N--L-SP---GLEY---NVSCAADSTIYASYYE
CGHGISTGGYGYSVP-ISDTIIPAV
IMABIS035
PPTDLR-F--T-NIG-P-----D--TMRVTWAASGYTIGPT-NFLVRYSPV------K--
N--EEDV--AELSINMGDNAVVL-T--N--L-LP---GTEY---VVSCAADSTIYASYYE
CGHGISTGGYGYSTPL-RGRQKTGL
IMABIS036
NPPHNL-S--V-INSEE-----LSSILKLTWTASGYTIGPL-KYNIQYRTKD-----A--
S--TW-S--QIPPENMGRSSFTV-Q--D--L-KP---FTEY---VFRCAADSTIYASYYE
CGHGISTGGYGYSDWSEEASGITYE
IMABIS037
PCGYIS-P--ESPVV-Q-----LHSNFTAVCVASGYTIGPN-YIVWKTN-----------
----H-F--TIPK-NMGASSVTF-T--D--I-AS---L-NI---QLTCAADSTIYASYYE
CGHGISTGGYGYEQNVYGITIISGL
IMABIS038
EKPKNL-S--CIVN--------EGKKMRCEWDASGYTIGPT-NFTLKSEWA------T--
H--KF----ADCKANMGPTSCTV-D--T--STVY---FVNI---EVWCAADSTIYASYYE
CGHGISTGGYGYKVTSDHINFDPVY
IMABIS039
RFIVKP-Y--G-TEV-G-----EGQSANFYCRASGYTIGPP-VVTWHKD-----------
D--RE-L--K----NMGDYGLTI-N--R--V-KG---DDKG---EYTCAADSTIYASYYE
CGHGISTGGYGYGTKEEIVFLNVTR
IMABIS041
SEPGRL-A--FNV---V-----SSTVTQLSWAASGYTIGPT-AYEVCYGLVNDDNRPI--
G--PM-K--KVLVDNMGNRMLLI-E--N--L-RE---SQPY---RYTCAADSTIYASYYE
CGHGISTGGYGYWGPEREAIINLAT
IMABIS042
APQNPN-A--K-AA--------GSRKIHFNWLASGYTIGPM-GYRVKYWIQ------G--
D--SE-SEAHLLDSNMGVPSVEL-T--N--L-YP---YCDY---EMKCAADSTIYASYYE
CGHGISTGGYGYGPYSSLVSCRTHQ
IMABIS044
IEVEKP--LYG-VEV-F-----VGETAHFEIEASGYTIGPV-HGQWKLKGQP--------
--------------NMGKHILIL-H--N--C-QL---GMTG---EVSCAADSTIYASYYE
CGHGISTGGYGY-NAKSAANLKVKE
IMABIS045
FKIETT-PESR-YLA-Q-----IGDSVSLTCSASGYTIGPP-FFSWRTQIDS--------
--------------NMGTSTLTM-N--P--V-SF---GNEH---SYLCAADSTIYASYYE
CGHGISTGGYGYRKLEKGIQVEIYS

TABLE 11
Four examples of six-stranded (strands-only) folds
in PDB 2.0 format to indicate spatial conformation.

| | | | | | | | | | | |
|---|---|---|---|---|---|---|---|---|---|---|
| 1NEU | | | | | | | | | | |
| ATOM | 1 | CA | GLY | 15 | −13.154 | 9.208 | −3.380 | 1.00 | 23.37 | C |
| ATOM | 2 | CA | GLY | 16 | −14.293 | 5.561 | −3.888 | 1.00 | 22.86 | C |
| ATOM | 3 | CA | GLY | 17 | −16.782 | 3.259 | −2.179 | 1.00 | 20.21 | C |
| ATOM | 4 | CA | GLY | 18 | −17.260 | −0.530 | −2.245 | 1.00 | 19.23 | C |
| ATOM | 5 | CA | GLY | 19 | −20.702 | −2.004 | −2.834 | 1.00 | 17.13 | C |
| ATOM | 6 | CA | GLY | 20 | −20.862 | −5.442 | −1.161 | 1.00 | 19.06 | C |
| ATOM | 7 | CA | GLY | 21 | −22.944 | −8.326 | −2.443 | 1.00 | 15.28 | C |
| ATOM | 8 | CA | GLY | 22 | −22.792 | −11.964 | −1.378 | 1.00 | 15.88 | C |
| ATOM | 9 | CA | GLY | 23 | −25.143 | −14.899 | −1.017 | 1.00 | 18.12 | C |
| ATOM | 10 | CA | GLY | 24 | −25.395 | −17.869 | 1.327 | 1.00 | 18.67 | C |
| ATOM | 11 | CA | GLY | 25 | −27.226 | −21.197 | 1.356 | 1.00 | 20.29 | C |
| ATOM | 12 | CA | GLY | 33 | −24.118 | −18.315 | −10.706 | 1.00 | 24.64 | C |
| ATOM | 13 | CA | GLY | 34 | −24.637 | −14.823 | −12.130 | 1.00 | 21.24 | C |
| ATOM | 14 | CA | GLY | 35 | −24.488 | −11.328 | −10.549 | 1.00 | 17.90 | C |
| ATOM | 15 | CA | GLY | 36 | −26.181 | −8.277 | −12.056 | 1.00 | 16.78 | C |
| ATOM | 16 | CA | GLY | 37 | −25.898 | −4.707 | −10.644 | 1.00 | 15.77 | C |
| ATOM | 17 | CA | GLY | 38 | −28.607 | −2.078 | −11.499 | 1.00 | 16.15 | C |
| ATOM | 18 | CA | GLY | 39 | −28.680 | 1.671 | −10.617 | 1.00 | 17.11 | C |
| ATOM | 19 | CA | GLY | 40 | −31.657 | 4.031 | −9.957 | 1.00 | 19.95 | C |
| ATOM | 20 | CA | GLY | 69 | −15.648 | 4.079 | −12.895 | 1.00 | 19.37 | C |
| ATOM | 21 | CA | GLY | 70 | −16.470 | 0.404 | −12.145 | 1.00 | 17.40 | C |
| ATOM | 22 | CA | GLY | 71 | −14.063 | −2.286 | −10.851 | 1.00 | 17.48 | C |
| ATOM | 23 | CA | GLY | 72 | −14.971 | −5.980 | −10.384 | 1.00 | 17.29 | C |
| ATOM | 24 | CA | GLY | 73 | −13.707 | −7.259 | −7.030 | 1.00 | 17.82 | C |
| ATOM | 25 | CA | GLY | 74 | −15.790 | −10.357 | −6.383 | 1.00 | 20.26 | C |
| ATOM | 26 | CA | GLY | 81 | −20.196 | −10.332 | −6.333 | 1.00 | 15.95 | C |
| ATOM | 27 | CA | GLY | 82 | −18.870 | −7.004 | −5.037 | 1.00 | 14.51 | C |
| ATOM | 28 | CA | GLY | 83 | −17.786 | −3.962 | −7.142 | 1.00 | 14.59 | C |
| ATOM | 29 | CA | GLY | 84 | −16.094 | −0.638 | −6.409 | 1.00 | 15.51 | C |
| ATOM | 30 | CA | GLY | 85 | −17.498 | 2.735 | −7.656 | 1.00 | 16.32 | C |
| ATOM | 31 | CA | GLY | 86 | −14.567 | 5.088 | −8.340 | 1.00 | 17.40 | C |
| ATOM | 32 | CA | GLY | 87 | −14.105 | 8.889 | −8.375 | 1.00 | 20.53 | C |
| ATOM | 33 | CA | GLY | 89 | −19.429 | 12.768 | −7.705 | 1.00 | 33.26 | C |
| ATOM | 34 | CA | GLY | 90 | −22.291 | 14.637 | −6.007 | 1.00 | 33.41 | C |
| ATOM | 35 | CA | GLY | 91 | −24.761 | 13.465 | −8.588 | 1.00 | 28.24 | C |
| ATOM | 36 | CA | GLY | 92 | −24.071 | 9.808 | −7.919 | 1.00 | 23.61 | C |
| ATOM | 37 | CA | GLY | 93 | −26.736 | 9.584 | −5.107 | 1.00 | 22.12 | C |
| ATOM | 38 | CA | GLY | 94 | −29.127 | 6.723 | −5.698 | 1.00 | 18.21 | C |
| ATOM | 39 | CA | GLY | 95 | −30.045 | 3.141 | −4.991 | 1.00 | 18.57 | C |
| ATOM | 40 | CA | GLY | 96 | −28.033 | 0.110 | −6.301 | 1.00 | 17.72 | C |
| ATOM | 41 | CA | GLY | 97 | −29.544 | −3.367 | −6.491 | 1.00 | 18.72 | C |
| ATOM | 42 | CA | GLY | 98 | −27.685 | −6.700 | −6.623 | 1.00 | 18.53 | C |
| ATOM | 43 | CA | GLY | 99 | −29.584 | −9.550 | −8.379 | 1.00 | 18.45 | C |
| ATOM | 44 | CA | GLY | 100 | −28.276 | −13.106 | −7.867 | 1.00 | 17.71 | C |
| ATOM | 45 | CA | GLY | 108 | −33.268 | −14.108 | −8.956 | 1.00 | 32.51 | C |
| ATOM | 46 | CA | GLY | 109 | −33.081 | −12.828 | −5.395 | 1.00 | 28.62 | C |
| ATOM | 47 | CA | GLY | 110 | −32.234 | −9.138 | −4.891 | 1.00 | 23.31 | C |
| ATOM | 48 | CA | GLY | 111 | −30.950 | −6.748 | −2.225 | 1.00 | 19.82 | C |
| ATOM | 49 | CA | GLY | 112 | −30.333 | −2.979 | −2.412 | 1.00 | 19.75 | C |
| ATOM | 50 | CA | GLY | 113 | −28.024 | −0.343 | −0.875 | 1.00 | 18.42 | C |
| ATOM | 51 | CA | GLY | 114 | −28.469 | 3.472 | −1.024 | 1.00 | 18.70 | C |
| ATOM | 52 | CA | GLY | 115 | −25.546 | 5.827 | −1.713 | 1.00 | 19.95 | C |
| ATOM | 53 | CA | GLY | 116 | −25.095 | 9.402 | −0.363 | 1.00 | 25.78 | C |
| ATOM | 54 | CA | GLY | 117 | −22.037 | 11.484 | −1.264 | 1.00 | 31.06 | C |
| ATOM | 55 | CA | GLY | 118 | −20.985 | 14.508 | 0.805 | 1.00 | 38.60 | C |
| ATOM | 56 | CA | GLY | 119 | −17.884 | 16.768 | 1.101 | 1.00 | 42.50 | C |
| TER | | | | | | | | | | |
| 1MEL | | | | | | | | | | |
| ATOM | 57 | CA | GLY A | 16 | −13.853 | 9.205 | −3.269 | 1.00 | 7.90 | C |
| ATOM | 58 | CA | GLY A | 17 | −14.557 | 5.500 | −3.346 | 1.00 | 7.54 | C |
| ATOM | 59 | CA | GLY A | 18 | −16.958 | 3.068 | −1.674 | 1.00 | 6.47 | C |
| ATOM | 60 | CA | GLY A | 19 | −17.168 | −0.701 | −1.485 | 1.00 | 5.43 | C |
| ATOM | 61 | CA | GLY A | 20 | −20.455 | −2.621 | −1.546 | 1.00 | 8.09 | C |
| ATOM | 62 | CA | GLY A | 21 | −20.823 | −6.351 | −0.794 | 1.00 | 8.76 | C |
| ATOM | 63 | CA | GLY A | 22 | −23.429 | −9.023 | −1.196 | 1.00 | 8.33 | C |
| ATOM | 64 | CA | GLY A | 23 | −23.620 | −12.484 | 0.211 | 1.00 | 13.77 | C |
| ATOM | 65 | CA | GLY A | 24 | −26.198 | −14.877 | −1.245 | 1.00 | 17.53 | C |
| ATOM | 66 | CA | GLY A | 25 | −27.419 | −17.241 | 1.448 | 1.00 | 23.23 | C |
| ATOM | 67 | CA | GLY A | 26 | −29.608 | −20.285 | 1.362 | 1.00 | 25.25 | C |
| ATOM | 68 | CA | GLY A | 32 | −25.105 | −18.522 | −11.770 | 1.00 | 5.00 | C |
| ATOM | 69 | CA | GLY A | 33 | −24.991 | −14.689 | −11.775 | 1.00 | 4.64 | C |
| ATOM | 70 | CA | GLY A | 34 | −24.729 | −11.897 | −9.152 | 1.00 | 2.48 | C |
| ATOM | 71 | CA | GLY A | 35 | −24.799 | −8.264 | −10.249 | 1.00 | 2.00 | C |
| ATOM | 72 | CA | GLY A | 36 | −25.716 | −4.753 | −9.249 | 1.00 | 3.40 | C |
| ATOM | 73 | CA | GLY A | 37 | −28.543 | −2.502 | −10.376 | 1.00 | 6.64 | C |
| ATOM | 74 | CA | GLY A | 38 | −29.128 | 1.074 | −9.379 | 1.00 | 8.74 | C |
| ATOM | 75 | CA | GLY A | 39 | −32.163 | 3.371 | −9.309 | 1.00 | 19.24 | C |
| ATOM | 76 | CA | GLY A | 67 | −14.374 | 3.095 | −12.462 | 1.00 | 12.68 | C |
| ATOM | 77 | CA | GLY A | 68 | −16.189 | 0.136 | −10.946 | 1.00 | 8.15 | C |
| ATOM | 78 | CA | GLY A | 69 | −14.842 | −3.360 | −10.453 | 1.00 | 8.39 | C |
| ATOM | 79 | CA | GLY A | 70 | −16.896 | −6.384 | −9.408 | 1.00 | 5.47 | C |
| ATOM | 80 | CA | GLY A | 71 | −15.309 | −9.468 | −7.894 | 1.00 | 8.48 | C |
| ATOM | 81 | CA | GLY A | 72 | −16.197 | −12.511 | −5.798 | 1.00 | 18.00 | C |
| ATOM | 82 | CA | GLY A | 79 | −19.282 | −10.422 | −4.331 | 1.00 | 9.55 | C |
| ATOM | 83 | CA | GLY A | 80 | −18.031 | −6.806 | −4.095 | 1.00 | 4.94 | C |
| ATOM | 84 | CA | GLY A | 81 | −18.236 | −3.718 | −6.133 | 1.00 | 4.92 | C |
| ATOM | 85 | CA | GLY A | 82 | −15.331 | −1.340 | −5.635 | 1.00 | 7.44 | C |
| ATOM | 86 | CA | GLY A | 83 | −16.455 | 2.094 | −6.795 | 1.00 | 6.55 | C |
| ATOM | 87 | CA | GLY A | 84 | −13.706 | 4.649 | −7.462 | 1.00 | 9.49 | C |
| ATOM | 88 | CA | GLY A | 85 | −13.919 | 8.136 | −8.959 | 1.00 | 12.54 | C |
| ATOM | 89 | CA | GLY A | 87 | −19.256 | 11.437 | −9.096 | 1.00 | 15.46 | C |
| ATOM | 90 | CA | GLY A | 88 | −22.580 | 12.828 | −7.836 | 1.00 | 13.94 | C |
| ATOM | 91 | CA | GLY A | 89 | −24.298 | 11.258 | −10.888 | 1.00 | 18.68 | C |
| ATOM | 92 | CA | GLY A | 90 | −23.577 | 7.916 | −9.175 | 1.00 | 9.55 | C |
| ATOM | 93 | CA | GLY A | 91 | −26.004 | 8.885 | −6.360 | 1.00 | 6.54 | C |
| ATOM | 94 | CA | GLY A | 92 | −28.681 | 6.180 | −6.349 | 1.00 | 6.38 | C |
| ATOM | 95 | CA | GLY A | 93 | −30.154 | 3.149 | −4.618 | 1.00 | 6.33 | C |
| ATOM | 96 | CA | GLY A | 94 | −27.980 | 0.121 | −5.274 | 1.00 | 6.08 | C |
| ATOM | 97 | CA | GLY A | 95 | −29.551 | −3.256 | −5.491 | 1.00 | 10.12 | C |
| ATOM | 98 | CA | GLY A | 96 | −27.885 | −6.624 | −5.451 | 1.00 | 10.76 | C |
| ATOM | 99 | CA | GLY A | 97 | −29.379 | −9.337 | −7.656 | 1.00 | 9.08 | C |
| ATOM | 100 | CA | GLY A | 98 | −28.752 | −13.014 | −8.455 | 1.00 | 8.28 | C |
| ATOM | 101 | CA | GLY A | 122 | −33.497 | −12.862 | −6.462 | 1.00 | 15.49 | C |
| ATOM | 102 | CA | GLY A | 123 | −33.164 | −9.374 | −5.211 | 1.00 | 12.37 | C |
| ATOM | 103 | CA | GLY A | 124 | −31.827 | −7.789 | −2.102 | 1.00 | 14.34 | C |
| ATOM | 104 | CA | GLY A | 125 | −32.483 | −4.741 | 0.002 | 1.00 | 22.49 | C |
| ATOM | 105 | CA | GLY A | 126 | −31.400 | −1.487 | −1.608 | 1.00 | 17.72 | C |
| ATOM | 106 | CA | GLY A | 127 | −28.513 | 0.495 | −0.221 | 1.00 | 16.51 | C |
| ATOM | 107 | CA | GLY A | 128 | −28.376 | 4.247 | −0.807 | 1.00 | 16.84 | C |
| ATOM | 108 | CA | GLY A | 129 | −25.116 | 5.718 | −1.910 | 1.00 | 11.90 | C |
| ATOM | 109 | CA | GLY A | 130 | −24.954 | 9.478 | −1.961 | 1.00 | 8.75 | C |
| ATOM | 110 | CA | GLY A | 131 | −22.066 | 11.329 | −3.496 | 1.00 | 14.37 | C |
| ATOM | 111 | CA | GLY A | 132 | −21.513 | 14.817 | −1.947 | 1.00 | 23.32 | C |
| ATOM | 112 | CA | GLY A | 133 | −20.378 | 18.169 | −3.369 | 1.00 | 36.14 | C |
| TER | | | | | | | | | | |
| 1F97 | | | | | | | | | | |
| ATOM | 113 | CA | GLY A | 43 | −13.239 | 8.515 | −3.437 | 1.00 | 14.44 | C |
| ATOM | 114 | CA | GLY A | 44 | −13.393 | 4.758 | −3.940 | 1.00 | 21.52 | C |
| ATOM | 115 | CA | GLY A | 45 | −16.246 | 2.710 | −2.546 | 1.00 | 22.48 | C |
| ATOM | 116 | CA | GLY A | 46 | −17.296 | −0.926 | −2.623 | 1.00 | 21.22 | C |
| ATOM | 117 | CA | GLY A | 47 | −21.001 | −1.717 | −2.638 | 1.00 | 20.06 | C |
| ATOM | 118 | CA | GLY A | 48 | −21.128 | −5.074 | −0.848 | 1.00 | 13.56 | C |
| ATOM | 119 | CA | GLY A | 49 | −23.434 | −7.972 | −1.703 | 1.00 | 19.14 | C |
| ATOM | 120 | CA | GLY A | 50 | −22.907 | −11.288 | 0.067 | 1.00 | 22.49 | C |
| ATOM | 121 | CA | GLY A | 51 | −24.848 | −14.490 | −0.589 | 1.00 | 20.58 | C |
| ATOM | 122 | CA | GLY A | 52 | −24.961 | −18.121 | 0.538 | 1.00 | 24.55 | C |
| ATOM | 123 | CA | GLY A | 53 | −26.625 | −21.271 | −0.752 | 1.00 | 17.82 | C |
| ATOM | 124 | CA | GLY A | 57 | −24.531 | −17.722 | −9.307 | 1.00 | 10.39 | C |
| ATOM | 125 | CA | GLY A | 58 | −25.219 | −15.054 | −11.903 | 1.00 | 8.00 | C |
| ATOM | 126 | CA | GLY A | 59 | −24.694 | −11.642 | −10.310 | 1.00 | 7.62 | C |
| ATOM | 127 | CA | GLY A | 60 | −26.312 | −8.537 | −11.794 | 1.00 | 6.84 | C |
| ATOM | 128 | CA | GLY A | 61 | −26.560 | −4.910 | −10.704 | 1.00 | 8.48 | C |
| ATOM | 129 | CA | GLY A | 62 | −28.971 | −2.156 | −11.641 | 1.00 | 13.92 | C |
| ATOM | 130 | CA | GLY A | 63 | −28.917 | 1.574 | −10.971 | 1.00 | 11.89 | C |
| ATOM | 131 | CA | GLY A | 64 | −32.146 | 3.463 | −10.346 | 1.00 | 23.18 | C |
| ATOM | 132 | CA | GLY A | 85 | −14.367 | 2.765 | −13.291 | 1.00 | 17.93 | C |
| ATOM | 133 | CA | GLY A | 86 | −16.308 | −0.184 | −11.839 | 1.00 | 15.44 | C |
| ATOM | 134 | CA | GLY A | 87 | −14.665 | −3.500 | −11.017 | 1.00 | 16.79 | C |
| ATOM | 135 | CA | GLY A | 88 | −16.581 | −6.711 | −10.341 | 1.00 | 14.07 | C |
| ATOM | 136 | CA | GLY A | 89 | −15.959 | −9.289 | −7.620 | 1.00 | 16.75 | C |
| ATOM | 137 | CA | GLY A | 91 | −18.578 | −9.954 | −2.969 | 1.00 | 17.13 | C |
| ATOM | 138 | CA | GLY A | 92 | −19.615 | −6.643 | −4.531 | 1.00 | 12.84 | C |
| ATOM | 139 | CA | GLY A | 93 | −18.746 | −3.911 | −7.017 | 1.00 | 12.33 | C |
| ATOM | 140 | CA | GLY A | 94 | −15.956 | −1.401 | −6.447 | 1.00 | 12.43 | C |
| ATOM | 141 | CA | GLY A | 95 | −16.021 | 2.137 | −7.822 | 1.00 | 11.12 | C |
| ATOM | 142 | CA | GLY A | 96 | −12.511 | 3.493 | −8.309 | 1.00 | 16.95 | C |
| ATOM | 143 | CA | GLY A | 97 | −14.158 | 6.925 | −7.885 | 1.00 | 21.11 | C |
| ATOM | 144 | CA | GLY A | 99 | −19.244 | 11.655 | −8.297 | 1.00 | 22.37 | C |
| ATOM | 145 | CA | GLY A | 100 | −22.479 | 13.329 | −7.197 | 1.00 | 23.75 | C |
| ATOM | 146 | CA | GLY A | 101 | −24.007 | 11.875 | −10.393 | 1.00 | 22.84 | C |
| ATOM | 147 | CA | GLY A | 102 | −23.793 | 8.420 | −8.818 | 1.00 | 15.51 | C |
| ATOM | 148 | CA | GLY A | 103 | −26.377 | 9.137 | −6.093 | 1.00 | 11.57 | C |
| ATOM | 149 | CA | GLY A | 104 | −29.205 | 6.618 | −6.153 | 1.00 | 9.52 | C |
| ATOM | 150 | CA | GLY A | 105 | −30.075 | 3.041 | −5.324 | 1.00 | 12.64 | C |
| ATOM | 151 | CA | GLY A | 106 | −28.157 | 0.010 | −6.540 | 1.00 | 7.32 | C |
| ATOM | 152 | CA | GLY A | 107 | −29.828 | −3.384 | −6.497 | 1.00 | 7.52 | C |
| ATOM | 153 | CA | GLY A | 108 | −27.747 | −6.545 | −6.374 | 1.00 | 9.97 | C |
| ATOM | 154 | CA | GLY A | 109 | −29.572 | −9.419 | −8.055 | 1.00 | 12.91 | C |
| ATOM | 155 | CA | GLY A | 110 | −28.245 | −12.921 | −7.462 | 1.00 | 11.27 | C |
| ATOM | 156 | CA | GLY A | 118 | −33.242 | −16.054 | −6.426 | 1.00 | 13.29 | C |
| ATOM | 157 | CA | GLY A | 119 | −32.582 | −13.085 | −4.179 | 1.00 | 15.55 | C |
| ATOM | 158 | CA | GLY A | 120 | −31.884 | −9.348 | −4.193 | 1.00 | 11.36 | C |
| ATOM | 159 | CA | GLY A | 121 | −30.813 | −6.471 | −1.975 | 1.00 | 11.36 | C |
| ATOM | 160 | CA | GLY A | 122 | −30.903 | −2.696 | −2.475 | 1.00 | 11.14 | C |
| ATOM | 161 | CA | GLY A | 123 | −28.232 | −0.209 | −1.404 | 1.00 | 11.58 | C |
| ATOM | 162 | CA | GLY A | 124 | −28.703 | 3.550 | −1.338 | 1.00 | 15.52 | C |
| ATOM | 163 | CA | GLY A | 125 | −25.598 | 5.531 | −2.225 | 1.00 | 9.61 | C |
| ATOM | 164 | CA | GLY A | 126 | −25.164 | 9.137 | −1.123 | 1.00 | 10.84 | C |
| ATOM | 165 | CA | GLY A | 127 | −22.043 | 10.899 | −2.383 | 1.00 | 10.58 | C |
| ATOM | 166 | CA | GLY A | 128 | −20.981 | 13.549 | 0.145 | 1.00 | 14.41 | C |
| ATOM | 167 | CA | GLY A | 129 | −20.447 | 17.126 | −1.025 | 1.00 | 12.32 | C |
| TER | | | | | | | | | | |
| 1DQT | | | | | | | | | | |
| ATOM | 168 | CA | GLY C | 14 | −9.505 | 5.982 | −6.825 | 1.00 | 30.82 | C |
| ATOM | 169 | CA | GLY C | 15 | −13.195 | 5.487 | −7.536 | 1.00 | 25.29 | C |
| ATOM | 170 | CA | GLY C | 16 | −13.507 | 1.942 | −6.167 | 1.00 | 21.63 | C |
| ATOM | 171 | CA | GLY C | 17 | −16.606 | 0.707 | −4.377 | 1.00 | 20.22 | C |
| ATOM | 172 | CA | GLY C | 18 | −16.814 | −2.817 | −3.021 | 1.00 | 20.86 | C |
| ATOM | 173 | CA | GLY C | 19 | −19.629 | −4.451 | −1.137 | 1.00 | 21.25 | C |
| ATOM | 174 | CA | GLY C | 20 | −21.383 | −7.769 | −0.574 | 1.00 | 21.73 | C |
| ATOM | 175 | CA | GLY C | 21 | −24.675 | −8.800 | −2.148 | 1.00 | 22.97 | C |
| ATOM | 176 | CA | GLY C | 22 | −26.301 | −11.552 | −0.160 | 1.00 | 26.59 | C |
| ATOM | 177 | CA | GLY C | 23 | −29.169 | −13.867 | −0.930 | 1.00 | 26.47 | C |
| ATOM | 178 | CA | GLY C | 24 | −31.335 | −16.565 | 0.573 | 1.00 | 31.54 | C |
| ATOM | 179 | CA | GLY C | 25 | −32.236 | −19.342 | 0.443 | 1.00 | 31.33 | C |
| ATOM | 180 | CA | GLY C | 32 | −26.091 | −17.451 | −10.077 | 1.00 | 21.25 | C |
| ATOM | 181 | CA | GLY C | 33 | −26.340 | −14.584 | −12.535 | 1.00 | 21.86 | C |
| ATOM | 182 | CA | GLY C | 34 | −25.686 | −11.294 | −10.762 | 1.00 | 19.78 | C |
| ATOM | 183 | CA | GLY C | 35 | −26.651 | −7.922 | −12.193 | 1.00 | 19.11 | C |
| ATOM | 184 | CA | GLY C | 36 | −25.711 | −4.438 | −10.995 | 1.00 | 19.23 | C |
| ATOM | 185 | CA | GLY C | 37 | −28.233 | −1.721 | −11.782 | 1.00 | 25.95 | C |
| ATOM | 186 | CA | GLY C | 38 | −27.978 | 2.010 | −11.165 | 1.00 | 30.90 | C |
| ATOM | 187 | CA | GLY C | 39 | −31.328 | 3.464 | −10.196 | 1.00 | 40.84 | C |
| ATOM | 188 | CA | GLY C | 66 | −16.664 | −4.410 | −12.490 | 1.00 | 23.17 | C |
| ATOM | 189 | CA | GLY C | 67 | −15.247 | −7.428 | −10.788 | 1.00 | 22.56 | C |
| ATOM | 190 | CA | GLY C | 68 | −16.273 | −9.875 | −8.095 | 1.00 | 21.60 | C |
| ATOM | 191 | CA | GLY C | 69 | −16.270 | −13.324 | −6.554 | 1.00 | 22.69 | C |
| ATOM | 192 | CA | GLY C | 75 | −21.405 | −12.287 | −4.112 | 1.00 | 23.03 | C |
| ATOM | 193 | CA | GLY C | 76 | −18.587 | −9.814 | −3.409 | 1.00 | 24.30 | C |
| ATOM | 194 | CA | GLY C | 77 | −18.961 | −6.951 | −5.886 | 1.00 | 23.46 | C |
| ATOM | 195 | CA | GLY C | 78 | −16.435 | −4.318 | −6.884 | 1.00 | 25.74 | C |
| ATOM | 196 | CA | GLY C | 79 | −17.349 | −1.380 | −9.129 | 1.00 | 26.08 | C |
| ATOM | 197 | CA | GLY C | 80 | −14.377 | 0.652 | −10.395 | 1.00 | 29.15 | C |
| ATOM | 198 | CA | GLY C | 81 | −13.639 | 3.807 | −12.365 | 1.00 | 26.23 | C |
| ATOM | 199 | CA | GLY C | 84 | −18.194 | 11.980 | −8.310 | 1.00 | 24.79 | C |
| ATOM | 200 | CA | GLY C | 85 | −21.048 | 12.151 | −10.804 | 1.00 | 25.37 | C |
| ATOM | 201 | CA | GLY C | 86 | −21.540 | 8.383 | −10.383 | 1.00 | 25.21 | C |
| ATOM | 202 | CA | GLY C | 87 | −22.696 | 8.922 | −6.809 | 1.00 | 23.95 | C |
| ATOM | 203 | CA | GLY C | 88 | −26.050 | 7.191 | −6.551 | 1.00 | 23.67 | C |
| ATOM | 204 | CA | GLY C | 89 | −28.103 | 4.091 | −5.812 | 1.00 | 22.75 | C |
| ATOM | 205 | CA | GLY C | 90 | −26.905 | 0.703 | −7.044 | 1.00 | 23.39 | C |
| ATOM | 206 | CA | GLY C | 91 | −29.167 | −2.332 | −7.030 | 1.00 | 22.55 | C |
| ATOM | 207 | CA | GLY C | 92 | −27.838 | −5.841 | −6.783 | 1.00 | 22.29 | C |
| ATOM | 208 | CA | GLY C | 93 | −29.956 | −8.462 | −8.555 | 1.00 | 20.45 | C |
| ATOM | 209 | CA | GLY C | 94 | −29.490 | −12.198 | −8.107 | 1.00 | 20.73 | C |
| ATOM | 210 | CA | GLY C | 105 | −34.479 | −11.361 | −6.201 | 1.00 | 24.94 | C |
| ATOM | 211 | CA | GLY C | 106 | −33.136 | −7.836 | −5.829 | 1.00 | 25.24 | C |
| ATOM | 212 | CA | GLY C | 107 | −31.842 | −5.752 | −2.931 | 1.00 | 24.05 | C |
| ATOM | 213 | CA | GLY C | 108 | −33.051 | −2.231 | −2.038 | 1.00 | 25.85 | C |
| ATOM | 214 | CA | GLY C | 109 | −29.830 | −0.739 | −3.310 | 1.00 | 23.77 | C |
| ATOM | 215 | CA | GLY C | 110 | −26.544 | 0.520 | −1.934 | 1.00 | 22.46 | C |
| ATOM | 216 | CA | GLY C | 111 | −25.963 | 4.285 | −1.818 | 1.00 | 24.13 | C |
| ATOM | 217 | CA | GLY C | 112 | −22.482 | 4.793 | −3.205 | 1.00 | 22.83 | C |
| ATOM | 218 | CA | GLY C | 113 | −20.958 | 8.192 | −2.417 | 1.00 | 23.40 | C |
| ATOM | 219 | CA | GLY C | 114 | −18.061 | 9.201 | −4.580 | 1.00 | 25.42 | C |
| END | | | | | | | | | | |

TABLE 12
PROSAII results (zp-comp) and values for the objective function
from MODELLER for 6-stranded iMab proteins. Lower values correspond
to iMab proteins that are more likely to fold correctly.

| imab nummer | objective funct. | zp-comb |
|---|---|---|
| iMabis050 | 617 | −1.83 |
| Mabis051 | 636 | −0.5 |
| iMab102 | 598 | −0.38 |
| Mabis052 | 586 | −0.88 |
| Mabis053 | 592 | −0.73 |
| Mabis054 | 540 | −0.42 |

TABLE 13

Example amino acid sequences likely to fold as six
stranded iMab proteins

iMab102
DDLKLTCRASGYTIGPYCMGWFRQAPNDDSTNVATINMGTVTLSMDDL
QPEDSAEYNCAADSTIYASYYECGHGLSTGGYGYDSHYRGT
iMabis050
DDLKLTSRASGYTIGPYCMGWFRQAPNDDSTNVATTNMGTVTLSMDDL
QPEDSAEYNSACDSTIYASYYECGHGLSTGGYGYDCRGQGT
iMabis051
DDLKLTSRASGYTIGPYCMGWFRQAPNDDSTNVATINMGTVTLSMDDL
QPEDSAEYNSCADSTIYASYYECGHGLSTGGYGYDSCGQGT
iMabis052
GSLRLSSAASGYTIGPYCMGWFRQAPGDDREGVAAINMGTVYLLMNSL
EPEDTAICYSAADSTIYASYYECGHGLSTGGYGYDSWGQGC
iMabis053
GSLRLSSAASGYTIGPYCMGWFRQAPGDDREGVAAINMGTVYLLMNSL
EPEDTAIYYSCADSTIYASYYECGHGLSTGGYGYDSCGQGT
iMabis054
GSLRLSSAASGYTIGPYCMGWFRQAPGDDREGVAAINMGTVYLLMNSL
EPEDTAIYYCAADSTIYASYYECGHGLSTGGYGYDSWGCGG

TABLE 14
PROSAII results (zp-comp) from iMab100 derivatives of which
lysine was replaced at either position 3, 7, 19 and 65 with
all other possible amino acid residues. Models were made with
and without native cysteine bridges. The more favourable derivatives
(which are hydrophilic) are denoted with X.

| results | without cysteine | with cysteine | |
|---|---|---|---|
| Residue replacement | bridges | bridges | |
| solubility | zp-comp | zp-comb | |
| IMABA_K_3_A | −6.61 | −6.85 | |
| IMABA_K_3_C | −6.72 | −6.62 | |
| IMABA_K_3_D X X | −6.65 | −6.54 | |
| IMABA_K_3_E X X | −6.63 | −6.48 | |
| IMABA_K_3_F | −6.61 | −6.44 | |
| IMABA_K_3_G | −6.70 | −6.63 | |
| IMABA_K_3_H | −6.70 | −6.79 | |
| IMABA_K_3_I | −6.65 | −6.47 | |
| IMABA_K_3_L | −6.42 | −6.55 | |
| IMABA_K_3_M | −6.34 | −6.57 | |
| IMABA_K_3_N X X | −6.57 | −6.41 | |
| IMABA_K_3_P | −6.74 | −6.46 | |
| IMABA_K_3_Q X X | −6.64 | −6.56 | |
| IMABA_K_3_R X X | −6.91 | −6.56 | best fit |
| IMABA_K_3_S X X | −6.52 | −6.61 | |
| IMABA_K_3_T X X | −6.69 | −6.61 | |
| IMABA_K_3_V | −6.60 | −6.63 | |
| IMABA_K_3_W | −6.61 | −6.57 | |
| IMABA_K_3_Y | −6.54 | −6.54 | |
| IMABA_K_7_A | −6.58 | −6.51 | |
| IMABA_K_7_C | −6.73 | −6.6 | |
| IMABA_K_7_D X X | −6.47 | −6.67 | |
| IMABA_K_7_E X X | −6.83 | −6.61 | |
| IMABA_K_7_F | −6.66 | −6.58 | |
| IMABA_K_7_G | −6.65 | −6.76 | |
| IMABA_K_7_H | −6.80 | −6.57 | |
| IMABA_K_7_I | −6.62 | −6.28 | |
| IMABA_K_7_L | −6.76 | −6.66 | |
| IMABA_K_7_M | −6.69 | −6.62 | |
| IMABA_K_7_N X X | −6.34 | −6.61 | |
| IMABA_K_7_P | −6.48 | −6.94 | |
| IMABA_K_7_Q X X | −6.72 | −6.61 | |
| IMABA_K_7_R X X | −6.63 | −6.94 | best fit |
| IMABA_K_7_S X X | −6.83 | −6.61 | |
| IMABA_K_7_T X X | −6.80 | −6.54 | |
| IMABA_K_7_V | −6.78 | −6.58 | |
| IMABA_K_7_W | −6.87 | −6.61 | |
| IMABA_K_7_W | −6.60 | −6.63 | |
| IMABA_K_19_A | −6.67 | −6.47 | |
| IMABA_K_19_C | −6.46 | −6.41 | |
| IMABA_K_19_D X X | −6.5 | −6.41 | |
| IMABA_K_19_E X X | −6.69 | −6.77 | |
| IMABA_K_19_F | −6.61 | −6.55 | |
| IMABA_K_19_G | −6.94 | −6.54 | |
| IMABA_K_19_H | −6.48 | −6.62 | |
| IMABA_K_19_I | −6.74 | −6.51 | |
| IMABA_K_19_L | −6.52 | −6.72 | |
| IMABA_K_19_M | −6.60 | −6.16 | |
| IMABA_K_19_N X X | −6.49 | −6.84 | |
| IMABA_K_19_P | −6.56 | −6.41 | |
| IMABA_K_19_Q X X | −6.91 | −6.65 | |
| IMABA_K_19_R X X | −6.72 | −6.73 | |
| IMABA_K_19_S X X | −6.57 | −6.61 | |
| IMABA_K_19_T X X | −6.85 | −6.61 | best fit |
| IMABA_K_19_V | −6.75 | −6.81 | |
| IMABA_K_19_W | −6.4 | −6.43 | |
| IMABA_K_19_Y | −6.53 | −6.48 | |
| IMABA_K_65_A | −6.52 | 6.22 | |
| IMABA_K_65_C | −6.43 | 6.23 | |
| IMABA_K_65_D X X | −6.79 | 6.72 | |
| IMABA_K_65_E X X | −6.82 | 6.70 | best fit |
| IMABA_K_65_F | −6.52 | 6.26 | |
| IMABA_K_65_G | −6.66 | 6.58 | |
| IMABA_K_65_H | −6.54 | 6.33 | |
| IMABA_K_65_I | −6.35 | 6.18 | |
| IMABA_K_65_L | −6.23 | 6.34 | |
| IMABA_K_65_M | −6.44 | 6.72 | |
| IMABA_K_65_N X X | −6.74 | 6.62 | |
| IMABA_K_65_P | −6.50 | 6.42 | |
| IMABA_K_65_Q X X | −6.62 | 6.59 | |
| IMABA_K_65_R X X | −6.63 | 6.53 | |
| IMABA_K_65_S X X | −6.68 | 6.41 | |
| IMABA_K_65_T X X | −6.47 | 6.41 | |
| IMABA_K_65_V | −6.30 | 6.25 | |
| IMABA_K_65_W | −6.50 | 6.39 | |
| IMABA_K_65_Y | −6.48 | 6.72 | |

TABLE 15
PROSAII results (zp-comp) from iMab100 derivatives
of which cysteine at position 96 was replaced with
all other possible amino acid residues.

| molecule | zp-comb |
|---|---|
| IMAB_C_96_A | −6.52 |
| IMAB_C_96_C | −7.11 |
| IMAB_C_96_D | −6.26 |
| IMAB_C_96_E | −5.75 |
| IMAB_C_96_F | −6.70 |
| IMAB_C_96_G | −6.38 |
| IMAB_C_96_H | −6.26 |
| IMAB_C_96_I | −6.66 |
| IMAB_C_96_K | −5.56 |
| IMAB_C_96_L | −6.37 |
| IMAB_C_96_M | −6.51 |
| IMAB_C_96_N | −6.53 |
| IMAB_C_96_P | −6.48 |
| IMAB_C_96_Q | −6.19 |
| IMAB_C_96_R | −6.08 |
| IMAB_C_96_S | −6.39 |
| IMAB_C_96_T | −6.38 |
| IMAB_C_96_V | −6.75 |
| IMAB_C_96_W | −6.22 |
| IMAB_C_96_Y | −6.60 |

Table 16

A) Amino acid sequence of iMab100 (reference) together with the possible candidates for extra cysteine bridge formation. The position where a cysteine bridge can be formed is indicated. B) Preferred locations for cysteine bridges with their corresponding PROSAII score (zp-comp) and the corresponding iMab name.

TABLE 16A

iMab 100 sequence

| | |
|---|---|
| 1 | NVKLVEKGGN FVENDDDLKL TCRAEGYTIG PYCMGWFRQA PNDDSTNVAT INMGGGITYY |
| 161 | GDSVKERFDI RRDNASNTVT LSMDDLQPED SAEYNCAGDS TIYASYYECG HGLSTGGYGY |
| 221 | DSHYRGQGTD VTVSS |

Possible candidiates:
CYS2_CYS24
CYS4_CYS22
CYS4_CYS111
CYS5_CYS24
CYS6_CYS22
CYS6_CYS112
CYS6_CYS115
CYS7_CYS22
CYS7_CYS115
CYS16_CYS84
CYS18_CYS82
CYS18_CYS84
CYS2O_CYS82
CYS21_CYS81
CYS22_CYS80
CYS23_CYS79
CYS34_CYS79
CYS35_CYS98
CYS36_CYS94
CYS39_CYS97
CYS37_CYS45
CYS37_CYS96
CYS38_CYS47
CYS38_CYS48
CYS39_CYS94
CYS92_CYS118
CYS94_CYS116
CYS95_CYS111
CYS95_CYS113
CYS95_CYS115
CYS98_CYS109
CYS98_CYS111
CYS99_CYS110

TABLE 16B
Preferred cysteine residues:

| Cysteine locations | zp-score | iMab name |
|---|---|---|
| CYS6 CYS112 | −7.81 | iMab111 |
| CYS35 CYS98 | −7.54 | |
| CYS99 CYS110 | −7.50 | |
| CYS5 CYS24 | −7.32 | |
| CYS23 CYS79 | −7.23 | |
| CYS38 CYS47 | −7.11 | iMab112 |

TABLE 17
Effect of mutation frequency of dITP on
the number of binders after panning

| Mutation | number of | number of |
|---|---|---|
| frequency | transformants | binders |
| 0 | 9.3*10E6 | 50 |
| 2 | 8.1*10E6 | 1000 |
| 3, 5 | 5.4*10E6 | 75 |
| 8 | 7.4*10E6 | 100 |
| 13 | 22*10E6 | 100 |

TABLE 18

Sequences of the vectors used in Example 40 and in Example
4.

CM114-IMAB100
AAGAAACCAATTGTCCATATTGCATCAGACATTGCCGTCACTGCGTCTTTTACTGGCTC
TTCTCGCTAACCAAACCGGTAACCCCGCTTATTAAAAGCATTCTGTAACAAAGCGGGA
CCAAAGCCATGACAAAAACGCGTAACAAAAGTGTCTATAATCACGGCAGAAAAGTCC
ACATTGATTATTTGCACGGCGTCACACTTTGCTATGCCATAGCATTTTTATCCATAAGA
TTAGCGGATCCTACCTGACGCTTTTTATCGCAACTCTCTACTGTTTCTCCATACCCGTTT
TTTGGGCTAACAGGAGAAGATATACCATGAAAAAACTGTTATTTGCGATTCCGCTGGT
GGTGCCGTTTTATAGCCATAGCGCGTGGCGGCCGCAATGTGAAACTGGTTGAAAAAGG
TGGCAATTTCGTCGAAAACGATGACGATCTTAAGCTCACGTGCCGTGCTGAAGGTTAC
ACCATTGGCCCGTACTGCATGGGTTGGTTCCGTCAGGCGCCGAACGACGACAGTACTA
ACGTGGCCACGATCAACATGGGTGGCGGTATTACGTACTACGGTGACTCCGTCAAAG
AGCGCTTCGATATCCGTCGCGACAACGCGTCCAACACCGTTACCTTATCGATGGACGA
TCTGCAACCGGAAGACTCTGCAGAATACAATTGTGCAGGTGATTCTACCATTTACGCG
AGCTATTATGAATGTGGTCATGGCCTGAGTACCGGCGGTTACGGCTACGATAGCCACT
ACCGTGGTCAGGGTACCGACGTTACCGTCTCGTCGGCCAGCTCGGCCGGTGGCGGTGG
CAGCTATACCGATATTGAAATGAACCGCCTGGGCAAAACCGGCAGCAGTGGTGATTC
GGGCAGCGCGTGGAGTCATCCGCAGTTTGAGAAAGCGGCGCGCCTGGAAACTGTTGA
AAGTTGTTTAGCAAAACCCCATACAGAAAATTCATTTACTAACGTCTGGAAAGACGAC
AAAACTTTAGATCGTTACGCTAACTATGAGGGTTGTCTGTGGAATGCTACAGGCGTTG
TAGTTTGTACTGGTGACGAAACTCAGTGTTACGGTACATGGGTTCCTATTGGGCTTGCT
ATCCCTGAAAATGAGGGTGGTGGCTCTGAGGGTGGCGGTTCTGAGGGTGGCGGTTCTG
AGGGTGGCGGTACTAAACCTCCTGAGTACGGTGATACACCTATTCCGGGCTATACTTA
TATCAACCCTCTCGACGGCACTTATCCGCCTGGTACTGAGCAAAACCCCGCTAATCCT
AATCCTTCTCTTGAGGAGTCTCAGCCTCTTAATACTTTCATGTTTCAGAATAATAGGTT
CCGAAATAGGCAGGGGGCATTAACTGTTTATACGGGCACTGTTACTCAAGGCACTGAC
CCCGTTAAAACTTATTACCAGTACACTCCTGTATCATCAAAAGCCATGTATGACGCTT
ACTGGAACGGTAAATTCAGAGACTGCGCTTTCCATTCTGGCTTTAATGAGGATCCATT
CGTTTGTGAATATCAAGGCCAATCGTCTGACCTGCCTCAACCTCCTGTCAATGCTGGC
GGCGGCTCTGGTGGTGGTTCTGGTGGCGGCTCTGAGGGTGGTGGCTCTGAGGGTGGCG
GTTCTGAGGGTGGCGGCTCTGAGGGAGGCGGTTCCGGTGGTGGCTCTGGTTCCGGTGA
TTTTGATTATGAAAAGATGGCAAACGCTAATAAGGGGGCTATGACCGAAAATGCCGA
TGAAAACGCGCTACAGTCTGACGCTAAAGGCAAACTTGATTCTGTCGCTACTGATTAC
GGTGCTGCTATCGATGGTTTCATTGGTGACGTTTCCGGCCTTGCTAATGGTAATGGTGC
TACTGGTGATTTTGCTGGCTCTAATTCCCAAATGGCTCAAGTCGGTGACGGTGATAAT
TCACCTTTAATGAATAATTTCCGTCAATATTTACCTTCCCTCCCTCAATCGGTTGAATG
TCGCCCTTTTGTCTTTAGCGCTGGTAAACCATATGAATTTTCTATTGATTGTGACAAAA
TAAACTTATTCCGTGGTGTCTTTGCGTTTCTTTTATATGTTGCCACCTTTATGTATGTAT
TTTCTACGTTTGCTAACATACTGCGTAATAAGGAGTCTTAAGGCGCGCCTGTAATGAA
CGGTCTCCAGCTTGGCTGTTTTGGCGGATGAGAGAAGATTTTCAGCCTGATACAGATT
AAATCAGAACGCAGAAGCGGTCTGATAAAACAGAATTTGCCTGGCGGCAGTAGCGCG
GTGGTCCCACCTGACCCCATGCCGAACTCAGAAGTGAAACGCCGTAGCGCCGATGGT
AGTGTGGGGTCTCCCCATGCGAGAGTAGGGAACTGCCAGGCATCAAATAAAACGAAA
GGCTCAGTCGAAAGACTGGGCCTTTCGTTTTATCTGTTGTTTGTCGGTGAACGCTCTCC
TGAGTAGGACAAATCCGCCGGGAGCGGATTTGAACGTTGCGAAGCAACGGCCCGGAG
GGTGGCGGGCAGGACGCCCGCCATAAACTGCCAGGCATCAAATTAAGCAGAAGGCCA
TCCTGACGGATGGCCTTTTTGCGTTTCTACAAACTCTTTTTGTTTATTTTTCTAAATACA
TTCAAATATGTATCCGCTCATGAGACAATAACCCTGATAAATGCTTCAATAATATTGA
AAAAGGAAGAGTATGAGTATTCAACATTTCCGTGTCGCCCTTATTCCCTTTTTTGCGGC
ATTTTGCCTTCCTGTTTTTGCTCACCCAGAAACGCTGGTGAAAGTAAAAGATGCTGAA
GATCAGTTGGGTGCACGAGTGGGTTACATCGAACTGGATCTCAACAGCGGTAAGATC
CTTGAGAGTTTTCGCCCCGAAGAACGTTTTCCAATGATGAGCACTTTTAAAGTTCTGCT
ATGTGGCGCGGTATTATCCCGTGTTGACGCCGGGCAAGAGCAACTCGGTCGCCGCATA
CACTATTCTCAGAATGACTTGGTTGAGTACTCACCAGTCACAGAAAAGCATCTTACGG
ATGGCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTTGCA
CAACATGGGGGATCATGTAACTCGCCTTGATCGTTGGGAACCGGAGCTGAATGAAGC
CATACCAAACGACGAGCGTGACACCACGATGCCTGTAGCAATGGCAACAACGTTGCG
CAAACTATTAACTGGCGAACTACTTACTCTAGCTTCCCGGCAACAATTAATAGACTGG
ATGGAGGCGGATAAAGTTGCAGGACCACTTCTGCGCTCGGCCCTTCCGGCTGGCTGGT
TTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAAGCA
TTGGTAACTGTCAGACCAAGTTTACTCATATATACTTTAGATTGATTTAAAACTTCATT
TTTAATTTAAAAGGATCTAGGTGAAGATCCTTTTTGATAATCTCATGACCAAAATCCCT
TAACGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGGTAACTG
GCTTCAGCAGAGCGCAGATACCAAATACTGTCCTTCTAGTGTAGCCGTAGTTAGGCCA
CCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTT
ACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGC
CACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCG
GAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTCA
GTGAGCGAGGAAGCGGAAGAGCGCCTGATGCGGTATTTTCTCCTTACGCATCTGTGCG
GTATTTCACACCGCATATGGTGCACTCTCAGTACAATCTGCTCTGATGCCGCATAGTTA
AGCCAGTATACACTCCGCTATCGCTACGTGACTGGGTCATGGCTGCGCCCCGACACCC
GCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGA
AACGCGCGAGGCAGCAGATCAATTCGCGCGCGAAGGCGAAGCGGCATGCATAATGTG
CCTGTCAAATGGACGAAGCAGGGATTCTGCAAACCCTATGCTACTCCGTCAAGCCGTC
AATTGTCTGATTCGTTACCAATTATGACAACTTGACGGCTACATCATTCACTTTTTCTT
CACAACCGGCACGGAACTCGCTCGGGCTGGCCCCGGTGCATTTTTTAAATACCCGCGA
GAAATAGAGTTGATCGTCAAAACCAACATTGCGACCGACGGTGGCGATAGGCATCCG
GGTGGTGCTCAAAAGCAGCTTCGCCTGGCTGATACGTTGGTCCTCGCGCCAGCTTAAG
ACGCTAATCCCTAACTGCTGGCGGAAAAGATGTGACAGACGCGACGGCGACAAGCAA
ACATGCTGTGCGACGCTGGCGATATCAAAATTGCTGTCTGCCAGGTGATCGCTGATGT
ACTGACAAGCCTCGCGTACCCGATTATCCATCGGTGGATGGAGCGACTCGTTAACGC
TTCCATGCGCCGCAGTAACAATTGCTCAAGCAGATTTATCGCCAGCAGCTCCGAATAG
CGCCCTTCCCCTTGCCCGGCGTTAATGATTTGCCCAAACAGGTCGCTGAAATGCGGCT
GGTGCGCTTCATCCGGGCGAAAGAACCCCGTATTGGCAAATATTGACGGCCAGTTAA
GCCATTCATGCCAGTAGGCGCGCGGACGAAAGTAAACCCACTGGTGATACCATTCGC
GAGCCTCCGGATGACGACCGTAGTGATGAATCTCTCCTGGCGGGAACAGCAAAATAT
CACCCGGTCGGCAAACAAATTCTCGTCCCTGATTTTTCACCACCCCCTGACCGCGAAT
GGTGAGATTGAGAATATAACCTTTCATTCCCAGCGGTCGGTCGATAAAAAAATCGAG
ATAACCGTTGGCCTCAATCGGCGTTAAACCCGCCACCAGATGGGCATTAAACGAGTAT
CCCGGCAGCAGGGGATCATTTTGCGCTTCAGCCATACTTTTCATACTCCCGCCATTCAG
AG
CM126-IMAB100
TTCTCATGTTTGACAGCTTATCATCGATAAGCTTTAATGCGGTAGTTTATCACAGTTAA
ATTGCTAACGCAGTCAGGCACCGTGTATGAAATCTAACAATGCGCTCATCGTCATCCT
CGGCACCGTCACCCTGGATGCTGTAGGCATAGGCTTGGTTATGCCGGTACTGCCGGGC
CTCTTGCGGGATATCGTCCATTCCGACAGCATCGCCAGTCACTATGGCGTGCTGCTAG
CGCTATATGCGTTGATGCAATTTCTATGCGCACCCGTTCTCGGAGCACTGTCCGACCG
CTTTGGCCGCCGCCCAGTCCTGCTCGCTTCGCTACTTGGAGCCACTATCGACTACGCG
ATCATGGCGACCACACCCGTCCTGTGGATATCCGGATATAGTTCCTCCTTTCAGCAAA
AAACCCCTCAAGACCCGTTTAGAGGCCCCAAGGGGTTATGCTAGTTATTGCTCAGCGG
TGGCAGCAGCCAACTCAGCTTCCTTTCGGGCTTTGTTAGCAGCCGGATCCTTAGTGGT
GATGGTGATGGTGGCTTTTGCCCAGGCGGTTCATTTCTATATCGGTATAGCTGCCACC
GCCACCGGCCGAGCTGGCCGACGAGACGGTAACGTCGGTACCCTGACCACGGTAGTG
GCTATCGTAGCCGTAACCGCCGGTACTCAGGCCATGACCACATTCATAATAGCTCGCG
TAAATGGTAGAATCACCTGCACAATTGTATTCTGCAGAGTCTTCCGGTTGCAGATCGT
CCATCGATAAGGTAACGGTGTTGGACGCGTTGTCGCGACGGATATCGAAGCGCTCTTT
GACGGAGTCACCGTAGTACGTAATACCGCCACCCATGTTGATCGTGGCCACGTTAGTA
CTGTCGTCGTTCGGCGCCTGACGGAACCAACCCATGCAGTACGGGCCAATGGTGTAAC
CTTCAGCACGGCACGTGAGCTTAAGATCGTCATCGTTTTCGACGAAATTGCCACCTTTT
TCAACCAGTTTCACATTCATATGTATATCTCCTTCTTAAAGTTAAACAAAATTATTTCT
AGAGGGAAACCGTTGTGGTCTCCCTATAGTGAGTCGTATTAATTTCGCGGGATCGAGA
TCTCGATCCTCTACGCCGGACGCATCGTGGCCGGCATCACCGGCGCCACAGGTGCGGT
TGCTGGCGCCTATATCGCCGACATCACCGATGGGGAAGATCGGGCTCGCCACTTCGGG
CTCATGAGCGCTTGTTTCGGCGTGGGTATGGTGGCAGGCCCCGTGGCCGGGGGACTGT
TGGGCGCCATCTCCTTGCATGCACCATTCCTTGCGGCGGCGGTGCTCAACGGCCTCAA
CCTACTACTGGGCTGCTTCCTAATGCAGGAGTCGCATAAGGGAGAGCGTCGATCGACC
GATGCCCTTGAGAGCCTTCAACCCAGTCAGCTCCTTCCGGTGGGCGCGGGGCATGACT
ATCGTCGCCGCACTTATGACTGTCTTCTTTATCATGCAACTCGTAGGACAGGTGCCGG
CAGCGCTCTGGGTCATTTTCGGCGAGGACCGCTTTCGCTGGAGCGCGACGATGATCGG
CCTGTCGCTTGCGGTATTCGGAATCTTGCACGCCCTCGCTCAAGCCTTCGTCACTGGTC
CCGCCACCAAACGTTTCGGCGAGAAGCAGGCCATTATCGCCGGCATGGCGGCCGACG
CGCTGGGCTACGTCTTGCTGGCGTTCGCGACGCGAGGCTGGATGGCCTTCCCCATTAT
GATTCTTCTCGCTTCCGGCGGCATCGGGATGCCCGCGTTGCAGGCCATGCTGTCCAGG
CAGGTAGATGACGACCATCAGGGACAGCTTCAAGGATCGCTCGCGGCTCTTACCAGC
CTAACTTCGATCACTGGACCGCTGATCGTCACGGCGATTTATGCCGCCTCGGCGAGCA
CATGGAACGGGTTGGCATGGATTGTAGGCGCCGCCCTATACCTTGTCTGCCTCCCCGC
GTTGCGTCGCGGTGCATGGAGCCGGGCCACCTCGACCTGAATGGAAGCCGGCGGCAC
CTCGCTAACGGATTCACCACTCCAAGAATTGGAGCCAATCAATTCTTGCGGAGAACTG
TGAATGCGCAAACCAACCCTTGGCAGAACATATCCATCGCGTCCGCCATCTCCAGCAG
CCGCACGCGGCGCATCTCGGGCAGCGTTGGGTCCTGGCCACGGGTGCGCATGATCGTG
CTCCTGTCGTTGAGGACCCGGCTAGGCTGGCGGGGTTGCCTTACTGGTTAGCAGAATG
AATCACCGATACGCGAGCGAACGTGAAGCGACTGCTGCTGCAAAACGTCTGCGACCT
GAGCAACAACATGAATGGTCTTCGGTTTCCGTGTTTCGTAAAGTCTGGAAACGCGGAA
GTCAGCGCCCTGCACCATTATGTTCCGGATCTGCATCGCAGGATGCTGCTGGCTACCC
TGTGGAACACCTACATCTGTATTAACGAAGCGCTGGCATTGACCCTGAGTGATTTTTC
TCTGGTCCCGCCGCATCCATACCGCCAGTTGTTTACCCTCACAACGTTCCAGTAACCG
GGCATGTTCATCATCAGTAACCCGTATCGTGAGCATCCTCTCTCGTTTCATCGGTATCA
TTACCCCCATGAACAGAAATCCCCCTTACACGGAGGCATCAGTGACCAAACAGGAAA
AAACCGCCCTTAACATGGCCCGCTTTATCAGAAGCCAGACATTAACGCTTCTGGAGAA
ACTCAACGAGCTGGACGCGGATGAACAGGCAGACATCTGTGAATCGCTTCACGACCA
CGCTGATGAGCTTTACCGCAGCTGCCTCGCGCGTTTCGGTGATGACGGTGAAAACCTC
TGACACATGCAGCTCCCGGAGACGGTCACAGCTTGTCTGTAAGCGGATGCCGGGAGC
AGACAAGCCCGTCAGGGCGCGTCAGCGGGTGTTGGCGGGTGTCGGGGCGCAGCCATG
ACCCAGTCACGTAGCGATAGCGGAGTGTATACTGGCTTAACTATGCGGCATCAGAGC
AGATTGTACTGAGAGTGCACCATATATGCGGTGTGAAATACCGCACAGATGCGTAAG
GAGAAAATACCGCATCAGGCGCTCTTCCGCTTCCTCGCTCACTGACTCGCTGCGCTCG
GTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAA
GATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTTAA
ATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGT
CGTGTAGATAACTACGATACGGGAGGGCTTACCATCTGGCCCCAGTGCTGCAATGATA
CCGCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAATT
GTTGCCGGGAAGCTAGAGTAAGTAGTTCGCCAGTTAATAGTTTGCGCAACGTTGTTGC
CATTGCTGCAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTTGCT
CTTGCCCGGCGTCAACACGGGATAATACCGCGCCACATAGCAGAACTTTAAAAGTGCT
CATCATTGGAAAACGTTCTTCGGGGCGAAAACTCTCAAGGATCTTACCGCTGTTGAGA
TCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATT
TATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTCTTCAA
GAA

### References

Altschul, S F., T L Madden, A A. Schaffer, J Zhang, Z Zhang, W Miller, and D J. Lipman Nucleic

Anonymous. FPLC purification of 6* His-tagged proteins from E. coli using Ni-NTA Superflow under native conditions. in QIAexpressionist™, Fifth edition, 83-84 (2001)

Acids Res. 25:3389-3402 (1997).

Bendahman N, Hamers R. Nature, 363(6428):446-8, (1993).

Berens S J, Wylie D E, Lopez O J. Int Immunol, 9(l):189-99, (1997).

Berman, H M, Westbrook, J., Feng, Z., Gilliland, G, Bhat, T N, Weissig, H., Shindyalov, I N, Boume P E: The Protein Data Bank. Nucleic Acids Research, 28 pp. 235-242 (2000)

Beste G, Schmidt F S, Stibora T, Skerra A. Proc Natl Acad Sci U S A., 96, 1898-1903, (1999).

Better M, Chang C P, Robinson R R, Horwitz AH. Science, 240(4855):1041-3, (1988)

Cadwell et al., PCR Methods Appl., 2, 28-33, (1992)

Crane L J, Tibtech, 8, 12-16 (1990)

Davies J, Riechmann L. FEBS Lett., 339(3):285-90, (1994)

Dimasi N, Martin F, Volpari C, Brunetti M, Biasiol G, Altamura S, Cortese R, De Francesco R, Steinkuhler C, Sollazzo M. J Virol. 1997 Oct;71(10):7461-9.

Gibrat, J F, Madej, T, Bryant, S H, Curr. Op. Struct. Biol., 6(3),377-385 (1996)Hamers-Casterman C, Atarhouch T, Muyldermans S, Robinson G, Hamers C, Songa E B,

Hooft, R W W, Vriend, G, Sander, C and Abola, E E. Nature 381, 272, (1996)

Holler P D, Kieke M C, Kranz D M, Wittrup K D, Nat. Biotechnol. 18(7): 754-759, (2000)

Holm, L and Sander, C. Nucl. Acids Res., 26, 316-319 (1998a)

Holm, L and Sander, C. Proteins, 33, 88-96 (1998b)

Koide S., Artificial antibody polypeptides, WO 98/56915, (1998)

Koide, U.S. Pat. No. 6,462,189.

Koide A, Bailey C W, Huang X, Koide S, J. Mol. Biol. 284 (4):1141-1151, (1998).

Kranz et al., PCT International Patent Publication WO 0148145

Ku J, Schultz P G. Proc Natl Acad Sci U S A. 92(14):6552-6, (1995)

Kuipers, Methods Mol Biol, 57 351-356, (1996)

Lauwereys M, Arbabi Ghahroudi M, Desmyter A, Kinne J, Holzer W, De Genst E, Wyns L, McConnell S J, Hoess R H. J Mol Biol., 250(4):460-70, (1995)

Laskowski, R A, MacArthur, M W, Moss, D S, and Thornton, J M. J. App. Cryst. 26 283 (1993)

Leung et al., Technique 1, 11-15, (1989)

Muyldermans S. EMBO J.,17(13):3512-20 (1998)

Murzin A. G et al. J. Mol. Biol., 247, 536-540 (1995)

Orengo C A, Jones D T, Thornton J M. Structure, 5(8) 1093-1108 (1997)

Rodger A. & Norden B. Circular dichroism and linear dichroism, Oxford University Press (1997)

Sanchez and Sali, Proc. Natl. Acad. Sci. USA, 95, 13597-13602 (1998)

Shindyalov and Boume Protein Engineering 11(9) 739-747, (1998)

Shusta et al. Nature. 362(6418):367-9, (1993)

Sippl,M J. Proteins, 17: 355-362 (1993)

Skerra A. Biochim Biophys Acta. October 18;1482(1-2):337-50, 2000.

Skerra A. J Biotechnol. June;74(4):257-75 (2001)

Skerra A, Pluckthun A. Science May 20;240(4855):1038-41, (1988)

Smith et al.. J Mol Biol. March 27;277(2):317-32 (1998)

Spee, J H, de Vos W M, Kuipers O P. Nucleic Acids Res 21(3):777-8 (1993)

Vriend, G. J. Mol. Graph. 8, 52--56 (1990)

Vu, K B, Ghahroudi, M. A., Wyns, L., Muyldernans, S. Mol. Immunol. 34, 1121-1131 (1997)

Xu et al., Biotechniques, 27, 1102-1108, (1999)

Zaccolo M, Williams D M, Brown D M, Gherardi E. J Mol Biol, 255(4):589-603 (1996).

What is claimed is:

1. An isolated proteinaceous molecule comprising:
a binding peptide; and
a core comprising a β-barrel of at least 4 strands, wherein said β-barrel comprises at least two β-sheets, wherein each of said at least two β-sheets comprises two of said at least 4 strands,
wherein said binding peptide connects two strands of said β-barrel and is outside a natural context of said binding peptide.

2. The isolated proteinaceous molecule of claim 1, wherein said β-barrel comprises at least 5 strands, wherein at least one of said at least two β-sheets comprises 3 strands of said at least 4 strands.

3. The isolated proteinaceous molecule of claim 1 or claim 2, wherein said β-barrel comprises at least 6 strands, wherein at least two of said at least two β-sheets comprises 3 strands of said at least 4 strands.

4. The isolated proteinaceous molecule of any one of claims 1-3, wherein said β-barrel comprises at least 7 strands, wherein at least one of said at least two β-sheets comprises 4 strands of said at least 4 strands.

5. The isolated proteinaceous molecule of any one of claims 1-4, wherein said β-barrel comprises at least 8 strands, wherein at least one of said at least two β-sheets comprises 4 strands of said at least 4 strands.

6. The isolated proteinaceous molecule of any one of claims 1-5, wherein said β-barrel comprises at least 9 strands, wherein at least one of said at least two β-sheets comprises 4 strands of said at least 4 strands.

7. The isolated proteinaceous molecule of any one of claims 1-6, wherein said binding peptide connects two strands of said β-barrel on an open side of said β-barrel.

8. The isolated proteinaceous molecule of any one of claims 1-7, wherein said binding peptide connects said at least two β-sheets of said β-barrel.

9. The isolated proteinaceous molecule of any one of claims 1-8, further comprising at least one other binding peptide.

10. The isolated proteinaceous molecule of any one of claims 1-9, comprising three binding peptides and three connecting peptide sequences.

11. The isolated proteinaceous molecule of any one of claims 1-9, comprising at least 4 binding peptides.

12. The isolated proteinaceous molecule of claim 11, wherein at least one binding peptide recognizes a target molecule other than at least one of the other binding peptides.

13. A process for identifying a proteinaceous molecule having an altered binding property, said process comprising:
introducing an alteration in the core of any one of the proteinaceous molecules of any one of claims 1-12; and
selecting the proteinaceous molecule having an altered binding property from said proteinaceous molecules.

14. A process for identifying a proteinaceous molecule having an altered structural property, said process comprising:
introducing an alteration in the core of any one of the proteinaceous molecules of any one of claims 1-12; and
selecting the proteinaceous molecule having an altered binding property from said proteinaceous molecules.

15. The process of claim 13 or claim 14, wherein said alteration comprises a post-translational modification.

16. The process of any one of claims 13-15 further comprising:
introducing a mutation into a nucleic acid encoding said proteinaceous molecule, wherein said mutation causes said alteration; and
expressing said nucleic acid in an expression system capable of producing said proteinaceous molecule.

17. A isolated proteinaceous molecule produced by the process of any one of claims 13-16.

18. The proteinaceous molecule of any one of claims 1-12 or 17, wherein said isolated proteinaceous molecule is of an immunoglobulin superfamily origin.

19. The isolated proteinaceous molecule of claim 18, wherein an exterior of the proteinaceous molecule is immunologically similar to a member of the immunoglobulin superfamily from which the proteinaceous molecule originates.

20. A cell comprising the isolated proteinaceous molecule of any one of claims 1-12 or 17-19.

21. A process for producing a nucleic acid encoding a proteinaceous molecule capable of displaying at least one desired peptide sequence, said process comprising:
providing a nucleic acid sequence encoding at least a first and second structural region separated by a second nucleic acid sequence encoding said at least one desired peptide sequence or a region where said second nucleic acid sequence can be inserted; and
mutating said nucleic acid sequence encoding said first and second structural regions to obtain the nucleic acid encoding said proteinaceous molecule capable of displaying at least one desired peptide sequence.

22. A process for displaying a desired peptide sequence, said process comprising: providing a nucleic acid encoding at least two β-sheets, said at least two β-sheets forming a β-barrel, wherein said nucleic acid comprises a region for inserting a sequence encoding said desired peptide sequence;
inserting a desired nucleic acid sequence encoding the desired peptide sequence into the region; and expressing said nucleic acid encoding said at least two β-sheets, wherein said at least two β-sheets comprise the first and second structural regions produced by the method according to claim 21.

23. A process for producing a library including artificial binding peptides, said process comprising:
providing at least one nucleic acid template, wherein each of said at least one nucleic acid templates encode different specific binding peptides;
producing a collection of nucleic acid derivatives by mutating said at least one nucleic acid templates; and
providing at least a portion of said collection to a peptide synthesis system to produce said library.

24. The process of claim 23, comprising providing at least two nucleic acid templates.

25. The process of claim 24, comprising providing at least 10 nucleic acid templates.

26. The process of any one of claims 23-25, wherein said nucleic acid derivatives are mutated by amplifying said at least one nucleic acid template with mutation prone nucleic acid amplification.

27. The process of claim 26, wherein said mutation prone nucleic acid amplification includes at least one non-degenerate primer.

28. The process of claim 27, wherein said at least one non-degenerate primer comprises a degenerate region.

29. The process of any one of claims 26-28, wherein said amplifying comprises at least one elongation step in the presence of dITP or dPTP.

30. The process of any one of claims 23-29, wherein said at least one nucleic acid template encodes the specific binding peptide having an affinity region comprising at least 14 amino acids.

31. The process of claim 30, wherein said affinity region comprises at least 16 amino acids.

32. The process of claim 31, wherein said affinity region comprises a length of about 24 amino acids.

33. The process of any one of claims 30-32, wherein said affinity region comprises at least 14 consecutive amino acids.

34. The process of any one of claims 23-33, wherein at least one of said at least one nucleic acid template encodes the proteinaceous molecule of any one of claims 1-12 or 17-19.

35. The process of any one of claims 23-34, further comprising:
providing a potential binding partner for at least one of said artificial binding peptides of said library; and
selecting the at least one of said artificial binding peptides capable of specifically binding to said binding partner from said library.

36. The process of claim 35, wherein said library is provided as a phage display library.

37. The isolated proteinaceous molecule of any one of claims 1-12 or 17-19, obtainable by the method according to claim 35 or the proteinaceous molecule comprising a peptide sequence selected from the group consisting of the peptide sequences illustrated in Table 2, 3, 10, 13 and 16.

38. A process for separating a substance from a mixture, said process comprising:
providing the proteinaceous molecule of any one of claims 1-12 or 17-19, or 37; and
binding the substance with the binding peptide of the proteinaceous molecule.

39. The process of claim 38, wherein said mixture is a biological fluid.

40. The process of claim 39, wherein said biological fluid is an excretion product of an organism.

41. The process of claim 40, wherein said excretion product is milk or originates from milk.

42. The process of claim 39, wherein said mixture is blood or originates from blood.

43. A pharmaceutical comprising the isolated proteinaceous molecule of any one of claims 1-12, 17-19, or 37.

44. A pharmaceutical formulation for treating a pathological condition involving unwanted proteins, cells or micro-organisms, said pharmaceutical composition comprising:
the proteinaceous molecule of any one of claims 1-12, 17-19 or 37.

45. A diagnostic assay comprising the isolated proteinaceous molecule of any one of claims 1-12, 17-19 or 37.

46. A gene delivery vehicle comprising:
the isolated proteinaceous molecule of any one of claims 1-12, 17-19 or 37; and
a gene of interest.

47. A gene delivery vehicle comprising:
a nucleic acid encoding the isolated proteinaceous molecule of any one of claims 1-12, 17-19 or 37; and
a nucleic acid sequence encoding a gene of interest.

48. The isolated proteinaceous molecule of any one of claims 1-12, 17-19 or 37 conjugated to a moiety of interest.

49. The isolated proteinaceous molecule of claim 48, wherein said moiety of interest is a toxic moiety.

50. A chromatography column comprising:
the isolated proteinaceous molecule of any one of claims 1-12, 17-19 or 37; and
a packing material.

51. A nucleic acid produced by a process, said process comprising:
providing a nucleic acid sequence encoding at least a first and second structural region separated by a second nucleic acid sequence encoding said at least one desired peptide sequence or a region where said second nucleic acid sequence can be inserted; and
mutating said nucleic acid sequence encoding said first and second structural regions to obtain the second nucleic acid encoding said proteinaceous molecule capable of displaying at least one desired peptide sequence.

52. A nucleic acid library comprising a collection of nucleic acids, said nucleic acids produced by the process according to claim 51.

53. The nucleic acid library of claim 52, further comprising a collection of nucleic acids encoding different affinity regions.

54. The nucleic acid library of claim 52 or 53, wherein said nucleic acid library is an expression library.

55. A proteinaceous molecule comprising a peptide sequence selected from the group consisting of the peptide sequences illustrated in Table 2, 3, 10, 13 and 16.
